SEQUENCE LISTING (102 sequences)

SEQ ID NO: 1 - DNA, 912 bp, Homo sapiens
  misc_feature: ribosomal protein L17 (RPL17), transcript variant 5
agcctgaggtgagtgtttctcctgcgttgctccgagggcccaatcctcctgccatcgccgccatcctggcttcgggggcgccggcctccaggcccccgggaggagaactcctagggctactaaatcctcgctggaggcggtggcttcttatgcgggaggacgtggcggagggcctgactttgggagccggggtcagtcggcctctgagggttgactggattggtgaggcccgtgtggctacttctgtggaagcagtgctgtagttactggaagataaaagggaaagcaagcccttggtgggggaaagtgatctgtgaaaatggttcgctattcacttgacccggagaaccccacgaaatcatgcaaatcaagaggttccaatcttcgtgttcactttaagaacactcgtgaaactgctcaggccatcaagggtatgcatatacgaaaagccacgaagtatctgaaagatgtcactttacagaaacagtgtgtaccattccgacgttacaatggtggagttggcaggtgtgcgcaggccaagcaatggggctggacacaaggtcggtggcccaaaaagagtgctgaatttttgctgcacatgcttaaaaacgcagagagtaatgctgaacttaagggtttagatgtagattctctggtcattgagcatatccaagtgaacaaagcacctaagatgcgccgccggacctacagagctcatggtcggattaacccatacatgagctctccctgccacattgagatgatccttacggaaaaggaacagattgttcctaaaccagaagaggaggttgcccagaagaaaaagatatcccagaagaaactgaagaaacaaaaacttatggcacgggagtaaattcagcattaaaataaatgtaattaaaaggaaaagaa

SEQ ID NO: 2 - protein, 184 aa, Homo sapiens [sequence omitted]

SEQ ID NO: 3 - DNA, 761 bp, Homo sapiens
  misc_feature: ribosomal protein L36a (RPL36A)
ctttctttccgcgccgatagcgctcacgcaagcatggttaacgtccctaaaacccgccggactttctgtaagaagtgtggcaagcaccaaccccataaagtgacacagtacaagaagggcaaggattctctgtacgcccagggaaagcggcgttatgacaggaagcagagtggctatggtgggcaaactaagccgattttccggaaaaaggctaaaactacaaagaagattgtgctaaggcttgagtgcgttgagcccaactgcagatctaagagaatgctggctattaaaagatgcaagcattttgaactgggaggagataagaagagaaagggccaagtgatccagttctaagtgtcatcttttattatgaagacaataaaatcttgagtttatgttcacttcatttgtttgctgttcatcttttgggagggaataagctagagccatcaatacaattccgcttgtggggaaatttatgcctcttactggtactacttgttttgcattgaagctgactggttgagttcacatcatatgttgcaattttctaatttggcacttcaatcactaggggccttatgaggcagtttgtcattatgcaatggttattggttatcatgtgagtagacacatttcaggctaatagggagaagtcagtaacacattcatagtgaatatgagatgtctttgctaagagttaagtgtcagatctttgttataacagttaatttaataaagaattttggcattgttcttca

SEQ ID NO: 4 - protein, 106 aa, Homo sapiens [sequence omitted]

SEQ ID NO: 5 - DNA, 622 bp, Homo sapiens
  misc_feature: thymosin beta 4 X-linked (TMSB4X)
aactcggtggtggccactgcgcagaccagacttcgctcgtactcgtgcgcctcgcttcgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagtttaaatgactgtgctgcccctttcacatcaaagaactactgacaacgaaggccgcgcctgcctttcccatctgtctatctatctggctggcagggaaggaaagaacttgcatgttggtgaaggaagaagtggggtggaagaagtggggtgggacgacagtgaaatctagagtaaaaccaagctggcccaaggtgtcctgcaggctgtaatgcagtttaatcagagtgccatttttttttttgttcaaatgattttaattattggaatgcacaatttttttaatatgcaaataaaaagtttaaaaa

SEQ ID NO: 6 - protein, 44 aa, Homo sapiens
  misc_feature: thymosin beta 4 X-linked (TMSB4X)
MSDKPDMAEIEKFDKSKLKKTETQEKNPLPSKETIEQEKQAGES

SEQ ID NO: 7 - DNA, 4712 bp, Homo sapiens
  misc_feature: coiled-coil domain containing 80 (CCDC80)
agccttctcactcctcactgagtccactctgaacgtgctaaaatgggaaggaggcggtgttttgctgatctgttaaattcttagtgaagtttccttgatttccagtggctgctgttgtttgagtttggtttggagcaaaactgaggtagtcctaacatttctgggactgaatccaggcaagagaaagaagaaaaagaagaagaaaaagaggaggaaaaaggtagggagaaataaagggaggagagaagcacagtgaaagaaaaaaaaagtcccttttcgacatcacattcctgtgttttccctcagcctggaaaacatattaatcccagtgcttttacgcccggaaacaaagagactaagccagactatgggggaaagggagataagaaggatcctggaactttaaagagggaaagagtgagattcagaaatcgccaggactggactttaagggacgtcctgtgtcagcacaagggactggcacacacagacacacgagaccgaggagaaactgcagacaaatggagatacaaagacttagaaggacagctcctttcacctcatcctacttgtccagaaggtaaaaagacacagccagaaagaaaaggcatcggctcagctctcagatcaggacaggctgtggatctgtggcggtactctgaaagctggagctgcagcacaccccttttgtattgctcaccctcggtaaagagagagagggctgggaggaaaagtagttcatctaggaaactgtcctgggaaccaaacttctgatttcttttgcaaccctctgcattccatctctatgagccaccattggattacacaatgacatggagaatgggaccccgtttcactatgctgttggccatgtggctagtgtgtggatcagaaccccacccccatgccactattagaggcagccacggaggacggaaagtgcctttggtttctccggacagcagtaggccagctcggtttctgaggcacactgggaggtctcgcggaattgagagatccactctggaggaaccaaaccttcagcctctccagagaaggaggagtgtgcccgtgttgagactagctcgcccaacagagccgccagcccgctcggacatcaatggggccgccgtgagacctgagcaaagaccagcagccaggggctctccgcgtgagatgatcagagatgaggggtcctcagctcggtcaagaatgttgcgtttcccttcggggtccagctctcccaacatccttgccagctttgcagggaagaacagagtatgggtcatctcagcccctcatgcctcggaaggctactaccgcctcatgatgagcctgctgaaggacgatgtgtactgtgagctggcggagaggcacatccaacagattgtgctcttccaccaggcaggtgaggaaggaggcaaggtgagaaggatcaccagcgagggccagatcctggagcagcccctggaccctagcctcatccctaagctgatgagcttcctgaagctggagaagggcaagtttggcatggtgctgctgaagaagacgctgcaggtggaggagcgctatccatatcccgttaggctggaagccatgtacgaggtcatcgaccaaggccccatccgtaggatcgagaagatcaggcagaagggctttgtccagaaatgtaaggcctctggtgtagagggccaggtggtggcggaggggaatgacggtggagggggagcaggaaggccaagcctgggcagcgagaagaagaaagaggacccaaggagagcacaagtcccaccaaccagagagagtcgggtgaaggtcctgagaaaactggccgccactgcaccagctttgccccaacctccctcaacccccagagccaccacccttcctcctgccccagccacaacagtgactcggtccacgtcccgggcggtaacagttgctgcaagacctatgaccaccactgcctttcccaccacgcagaggccctggaccccctcaccctcccacaggccccctacaaccactgaggtgatcactgccaggagaccctcagtttcagagaatctttaccctccatcccggaaggatcagcacagggagaggccacagacaaccaggaggcccagcaaggccaccagcttggagagcttcacaaatgcccctcccaccaccatctcagaacccagcacaagggctgctggcccaggccgtttccgggacaaccgcatggacaggcgggaacatggccaccgagacccaaatgtggtgccaggtcctcccaagccagcaaaggagaaacctcccaaaaagaaggcccaggacaaaattcttagtaatgagtatgaggagaagtatgacctcagccggcctactgcctctcagctggaggacgagctgcaggtggggaatgttccccttaaaaaagcaaaggagtctaaaaagcatgaaaagcttgagaaaccagagaaggagaagaaaaaaaagatgaagaatgagaacgcagacaagttacttaagagtgaaaagcaaatgaagaagtctgagaaaaagagcaagcaagagaaagagaagagcaagaagaaaaaaggaggtaaaacagaacaggatggctatcagaaacccaccaacaaacacttcacgcagagtcccaagaagtcagtggccgacctgctggggtcctttgaaggcaaacgaagactccttctgatcactgctcccaaggctgagaacaatatgtatgtgcaacaacgtgatgaatatctggaaagtttctgcaagatggctaccaggaaaatctctgtgatcaccatcttcggccctgtcaacaacagcaccatgaaaatcgaccactttcagctagataatgagaagcccatgcgagtggtggatgatgaagacttggtagaccagcgtctcatcagcgagctgaggaaagagtacggaatgacctacaatgacttcttcatggtgctaacagatgtggatctgagagtcaagcaatactatgaggtaccaataacaatgaagtctgtgtttgatctgatcgatactttccagtcccgaatcaaagatatggagaagcagaagaaggagggcattgtttgcaaagaggacaaaaagcagtccctggagaacttcctatccaggttccggtggaggaggaggttgctggtgatctctgctcctaacgatgaagactgggcctattcacagcagctctctgccctcagtggtcaggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgattcgatgcaacttcggagacaggaaatggcgattcagcagtcactggggatgcgctgcccagaagatgagtatgcaggctatggttaccatagttaccaccaaggataccaggatggttaccaggatgactaccgtcatcatgagagttatcaccatggatacccttactgagcagaaatatgtaaccttagactcagccagtttcctctgcagctgctaaaactacatgtggccagctccattcttccacactgcgtactacatttcctgcctttttctttcagtgtttttctaagactaaataaatagcaaactttcacctattcatgagttattattgaaacctcaaatcataaagacatttaaaagaattgtttttctaactggaggggctctagtgctaaataatagtactgaaaattgatattattttccttttcttatatgaaggaccttatttggcatataaaattttataaaatatgtatttaaagctttttcttattttttgtattaattggtaagtgaaaactctgttaaagatcacaccacaatgttttcaagaaacatctgaaaagataaaacaaagaacaaataacttataatacttacttaaattgacactttttgaaatgccagtctgaaaataattaagatatctctgctttgtatgagtttcttttatgaaacttgataccacgggagtccagtaatattggccacaaaagccagagaaagtaccaagcccagctttgttatcatagccacttcctgccctgcttctgttatttttagtgttttttcagatataaatcggggtccaggaaatcctcaccagaatctggcactgcagccaaaggcgatacttccagagttctagtaggctgctatggaatttctggcatgaaaattcttgacccctcacactttaccccctgtacagcacaggcataccatggagatattacaggatcagttccagaccaccataataaagtggatatcgcaataaagtgagtcacacaaaatttttggtttctcagtgcatataaaagttatgtttacactctttagtagtctattaagtgtataatagctttatgtccaaaaaatgtacatgttttattttaaaaatactttattgctaaaaattgctaatgatcataatctttttgcattgatgttgatggctgctgaatgat

SEQ ID NO: 8 - protein, 950 aa, Homo sapiens
  misc_feature: coiled-coil domain containing 80 (CCDC80)
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 9 - DNA, 871 bp, Homo sapiens
  misc_feature: ferritin light chain (FTL)
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgca

SEQ ID NO: 10 - protein, 175 aa, Homo sapiens
  misc_feature: ferritin light chain (FTL)
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 11 - DNA, 7573 bp, Homo sapiens
  misc_feature: ribosomal protein L37 (RPL37)
ctcttccggtctttctggtctcggccgcagaagcgagatgacgaagggaacgtcatcgtttggaaagcgtcgcaataagacgcacacgttgtgccgccgctgtggctctaaggcctaccaccttcagaagtcgacctgtggcaaatgtggctaccctgccaagcgcaagagaaagtataactggagtgccaaggctaaaagacgaaataccaccggaactggtcgaatgaggcacctaaaaattgtataccgcagattcaggcatggattccgtgaaggaacaacacctaaacccaagagggcagctgttgcagcatccagttcatcttaagaatgtcaacgattagtcatgcaataaatgttctggttttaaaaaatacatatctggttttggtaaggtatttttaatcaattaggcttgtagtatcagtgaaatactgtaggtttagggactgggctagcttcatatcagatttacttgttaagtgactgttttggaatgtttacttttggactgggtttgtaacacggttaaaggcaatgagaaacaagcagaattccaggagtccttgaagcagagggcactggaagacaatatagcagattaaaatagcacagctcatgtggcataggtgggtattttagatgtttgagtaaatttgaaagagtatgatgtttaaattacctttagcaacatgttcatctgctatgctgtcatgactagggggatgattattagtcacatagagcttgggagtaccactggaaacgtatgggtaggagtttaggtggcttctgtttttcaaaagatgatcttatcctagtatctgtaatgctcacttggcacacctgacttgtgggctgtgtgtaaggtggctagctaagtgaaaaaagcctgctaggtgtgagtcaacttaagaatatgtaaataggtttgagaaaaagtagggcttgggtgcaagtaaagattgagcaggaaataaaggaaaatcaagtataatccctgagatttgtagactaaaggcaatgatgtgggactacttggtcgaatttttttagccctcaacttggtaattgggtgtttctgtgttaaagcactgaaacttgctgtcgtgccttcctagttttcgtggtttattgacagggttgggggttttttttgtttttttaaaatgaagggacaaagtcaactggactgctgagtgagagggcaggggcagttgaagggaacatgaattgctggaacagctacataaaatagtgatgtagccaagtcatgctatttaaattataattctccactgtgtttagaataacatctgaggttcttaacctggccttggaagggtatcacttttacttgtaacctggaatggctttataatgtgctagctaattgctactctcatcttgtattttaactcctaatttacccttcaggtctcagcttcagaacattcacttataaagaaaccctgctgattaaatctctcttgggcttcctcccgaaatgtgagactatactttaaagatgtatggttagagtccaattgccattgcctttcttgtttacagatagtgcaatggcgcaatcctggctcactgcagcctctaattcctgggctcaagtcaagtggttctcctgcctcagccttctgagtagctgggactacagatgcacaccaccacacctggctaatttttgtgttttttgtagagatggagattcactcgactaattctttttgtattcttagtagagactgggtttcaacatgttgaccaggctggtctcgaactcgacctcgtgatccgccgcctcggcctcccaaagtgctaggattacaggcgtgagccaccactcccggctgtcatcatcaaattttcaagtgaagatagtctgttgaagattgaacaatgaccttgaaagacagctgagttgctgtgggtataatgtaaagctggtgaagtttggccagtttgggcttcagaagtctaagtctagtgaaggtaccctgacccccatataaacaacccttgaggccggtgtggtggctcacggctgtaatcctggcactttgggaggccgaggtgggcggatcacaaggtcaagagatcgagatcaccctggcccacatggtgagaccccaactctactaaaaatacaaaaattagctgggtgtggtggcatgtgcctgtagtcttagctacttgagaggctgaggcaggagaatcgcttgaacctgggaggcagaggttgcagtgagctgagatcatgccactgtgctccagcctggtggcacagtgagactccacctcaaaaaaaaaaaaaaatccttgaactaatgactcaaattttcaaatgaaacaaaataagcagtggatcttgcattggagatggagttaaactatgttgcccaggctggtcttggactcctggtttcaagtgatcctctcatcttggcctcccaaagtgctgggattacagggacgagccaccacaccccacctattgtctatatttctatctttaacagcacttcagtcctgccttaagttatagttatgtatagatacccattatactttaaatttttcagcagaaattatgcttttatcttctctgcagtgttatatgttggtgtgcaaaaatgttaaatttatttttcctaagtaccccatctgcttttcaactctgtcctctgcctgaaaagcctccctccagcccctacttccctcccatcttagttcacaaagtcaggttgatttgcccccagctgtcaaagcagactacctgtttccacatgtaactggttatgttctgtaaagttacaaaaatagaaaggttgaatctgtggcggccgggtacggtagctcacgtcctatagtcgcagcactttgggaggccaaggtgggcagttcacgtgaggtggggagttcgagaccagcctgaccaacatggagaaaccccgtctctactaaaaatacaaaaatattagccgggcgtggtggtgcatgcctgtaatcccagctactagggaggctgaggcaggagaattgcttgaacctgggagacggaggttgcggtgagctgcagaggcctcacctctaattgagacacaattatatattgttgatatatatatatatatatatacacacacacacacacactatgatggataaatgcatgagtttctgtgagagcattggaaaggagtttgtcactcaataggtgaagccaggctaagatttaagctgagccagggaggacttgaaggaatcatgatgagagagaaggtaagtggctttgccagcaatgaaacagctgacataatggtaaccagtcagaggagggcataactatgaaactggacaccttggttgtcaggttagaaggatggggtgtagggttggtaagaaaagaattcagggaagagcagcgatcagattatgaagaatttgtcttgagaaattacagaggatttaaaccagaatgttaggaatagttattctagcaagatgaatgtggaaagtgttagtgtgcatgtgatgagtcttgaagctggaaactaggtaacaggttcttaaatagttcatgtgaaaatcatgacagactaaggcagtggctgtggggctgtccgggagttctctacagaaaacatctaaaacttgaatgtgcaagtgagtagctaacttccaagcttcccatttctgtataatttaagcatgaaaatgagaacactgagatttgataggcatgtagaagtcagagtaagcaagagggcttgagttcatcgatgaacttcagtaactatccttgacttagtattggtggaaaccatttgtgaatttacagactccaaaaacaaaaagataatttagagtctcaaagtaagatttggggagatgtccattggagacagaagtggagagagggagaagttcatttgttgacatatttatgtatggtgcacctactgtgtgccaagccatgttagagatacagtgggagcaaaaccagatgtgttttctgccctctgactccagtggttgcagaattagcccaggaactagaattggatggagtcacacaaaccaaggaaggggattgtttgaggggggcactcagtcacatgtggctgaggccaaagagaatgaagatatattttaaaattctggatttggcaaaatttaggctagtctttacttttcataaacctctctcatgttagagcagcaaagaaaaaccagtttgccaagaggtcacgagattgggcagtgaggaatggggagtttagtttaaaagacaacactagatacttttggaagtttacctattctgtggttcccttttattgactaaatggtgcccttcattcatgaagcaagtatctatttagtgactactatgtgtcaaaagctatctaggcccagaaacttttaagtggaaaacaaaacaaagaccctgccctgacagatttcaatcatgtatatactgtatgtatgtttgtggaaggtgacataatagacaattgcagatagtgataagtgtgtaagacaaggataccacgagcgtaatagacagtggggatggaggaacctttagaaggttggttaagttgaatgataaggagccattcttttgtctttacctgaatgacttatcattcagccctttttgagttttgggttgcttgcagagtttaaccttgcctgtaattgaaacttaattttgtaatagcataacttcatgtaagaaaagcaaagcaatacattacagaattattttactgaagaacttgtttcagagaaagaggctgtttcacatttattagcacatttggattatatttaggtgttttatttttttttaaacaaaggagtttggatcataatacaagagaagcacagggcaaagacactgcataacctcaagaactaagaatggaaggactggccaggcctggtggtgcacacctgtaatcccagctactcaggaggctgaggcgagagaatcgcttgagcctggggggcagaggttgcagtgagctgagagaacggcgggcggcagagcgagactccatctcaaaaaaaaaaaaggataaaggaaggacttaagcaaaatcttccttgtaagtagaaggatgttttgacaagaaaagttgcaatggaaaaatggttctcatgtacacgagtatgtagaataagcatcgtgtgtggattggattcagatcaaaacattgcttttatgtttgtgtctttatacggtgggagtataccctggtgccccaggatgaagacttgacctgacccatgtatttttagattactcacagataacaaaaagtattttcatcatgattagttgcgaaaacagttttatttcaataggtaaaacgtgcagtcctatgtaatcgtcagaaggtaatcttaattatagcttgggtgtgctttaaactgcaagctggcagtggagggcacgattcctctgatttcagctttctccttatacttttctggagctgtgagctgcaagttaactcagtgggattaaagtgtagactggaggtacaaaaggtgaggagtgaggagatagggtagttcttccttggctggctggcttcataatccctgggccccgcagataattaaatcgactttttctgtctcaggcatttgtatgacctctttggaggttccctgctgggtagttatccttgtatctgatggacccatctcaatttaaaatactctgccaggttcggaggttcatgcttgtcatcccagcactttgggaggctcagaggtgccattggcttgagcccaagagtttgagaccagcctgggcaacctggtgaaacctcttctccattaaaaatacaaaaaattagccaggcatggtggtatgtgcctgtagtcccagctactcagggagctgaggtggaaggattgcttgagcctgggaggtcaaggctgcagtgaaccttgattgtgccactgcactccagcctggatgacaaagaccctgtctcaagaaaagtctgtaattctttcctaacccttagtatccagcctcagtcctgaggttttctttacctctgggggctattttatgccggctctctcctgagtgtcacacatctggtcctcaggaaacattctcacatccctggcctgaaaaaaacaatttcagggagatcgcatggcagcagccctctctgggctccccagctaaatggttgtacgaaacacatttcaaagctctctgaagggcttccttgaagttcctttcactgggtttcaaagtagaaggtagtaactcctttgtccaagaaggctgaattgagcacttaacaattctccaaagaaatttcttcattggtctctatcctagaccccttctgtatccttgatatggctgaggattctaaaaaatgaccagtcttacatgggagggctgggatataaaaaataaaataataaataatgataaaaactgaaaactgaccacgttcttggatatgttttctttgggttgtgtgtgtgtgtgagacttttgatagttacccaaagtaggaaaaatcccattctaataaggttatatttatgtagctctgcaaataaacatctagcaaatgtaaaaagtattttctttgccttaaaaatgattaaaattatttgaactcctgaggagtgttatatgaataaaattagtaagttatttggaggaaagttattttttaaaaagacaactggtaaaacagtacaggagaaaggccagcttcctcaagtgaggacagttgtttagaattgactgaggagcggccgggtgcggaggctcacatctgtaatcccacacgccttgggaggctctgaggcgggtagatcacctgaggtcaggagtttgagaccagcctggccaacatggcgaaaccccgtctccactaaaaatacaaaaattagccaggtgcagtggcacacacctgtaatcccagctactccgaaggctgaggcaggaggatcacctgagcccaggaagttgagactgcagtgagctgagattgcaccactgcactccagcctcagtgacagcgagactgtctcaaaaaaagaaaaaagtgactgaggaggaagaggccaggtggcaaatggaacagaatcaccaaagggtgaacaggactaaggcaatgtagtgtatggctcagctacgtcagagtggaaaaggtgttattagagcagaaactatggtccctgcgtcacagggaagcaacctacagagaagcagcagctccccaagagaggagagataagaagccagaagcctcagagtgaacaattgtcctattagggattgctctagagagagaaacctctgggaacgtacccctgtgaggcagcacagcacaatgcttttagaattgtatgagagttgatatatctccatttgttttgcaaaggcaaaaactaagatacagagattatctgttaaagttatgtatttctttggtaataaagatgctgacagtgttgctggaatgcatttctttaataaagatattgtacaatgaaa

SEQ ID NO: 12 - protein, 97 aa, Homo sapiens [sequence omitted]

SEQ ID NO: 13 - DNA, 1554 bp, Homo sapiens
  misc_feature: heme oxygenase 1 (HMOX1)
aacgcctgcctcctctcgagcgtcctcagcgcagccgccgcccgcggagccagcacgaacgagcccagcaccggccggatggagcgtccgcaacccgacagcatgccccaggatttgtcagaggccctgaaggaggccaccaaggaggtgcacacccaggcagagaatgctgagttcatgaggaactttcagaagggccaggtgacccgagacggcttcaagctggtgatggcctccctgtaccacatctatgtggccctggaggaggagattgagcgcaacaaggagagcccagtcttcgcccctgtctacttcccagaagagctgcaccgcaaggctgccctggagcaggacctggccttctggtacgggccccgctggcaggaggtcatcccctacacaccagccatgcagcgctatgtgaagcggctccacgaggtggggcgcacagagcccgagctgctggtggcccacgcctacacccgctacctgggtgacctgtctgggggccaggtgctcaaaaagattgcccagaaagccctggacctgcccagctctggcgagggcctggccttcttcaccttccccaacattgccagtgccaccaagttcaagcagctctaccgctcccgcatgaactccctggagatgactcccgcagtcaggcagagggtgatagaagaggccaagactgcgttcctgctcaacatccagctctttgaggagttgcaggagctgctgacccatgacaccaaggaccagagcccctcacgggcaccagggcttcgccagcgggccagcaacaaagtgcaagattctgcccccgtggagactcccagagggaagcccccactcaacacccgctcccaggctccgcttctccgatgggtccttacactcagctttctggtggcgacagttgctgtagggctttatgccatgtgaatgcaggcatgctggctcccagggccatgaactttgtccggtggaaggccttctttctagagagggaattctcttggctggcttccttaccgtgggcactgaaggctttcagggcctccagccctctcactgtgtccctctctctggaaaggaggaaggagcctatggcatcttccccaacgaaaagcacatccaggcaatggcctaaacttcagagggggcgaagggatcagccctgcccttcagcatcctcagttcctgcagcagagcctggaagacaccctaatgtggcagctgtctcaaacctccaaaagccctgagtttcaagtatccttgttgacacggccatgaccactttccccgtgggccatggcaatttttacacaaacctgaaaagatgttgtgtcttgtgtttttgtcttatttttgttggagccactctgttcctggctcagcctcaaatgcagtatttttgttgtgttctgttgtttttatagcagggttggggtggtttttgagccatgcgtgggtggggagggaggtgtttaacggcactgtggccttggtctaacttttgtgtgaaataataaacaacattgtctgatagta

SEQ ID NO: 14 - protein, 288 aa, Homo sapiens
  misc_feature: heme oxygenase 1 (HMOX1)
MERPQPDSMPQDLSEALKEATKEVHTQAENAEFMRNFQKGQVTRDGFKLVMASLYHIYVALEEEIERNKESPVFAPVYFPEELHRKAALEQDLAFWYGPRWQEVIPYTPAMQRYVKRLHEVGRTEPELLVAHAYTRYLGDLSGGQVLKKIAQKALDLPSSGEGLAFFTFPNIASATKFKQLYRSRMNSLEMTPAVRQRVIEEAKTAFLLNIQLFEELQELLTHDTKDQSPSRAPGLRQRASNKVQDSAPVETPRGKPPLNTRSQAPLLRWVLTLSFLVATVAVGLYAM

SEQ ID NO: 15 - DNA, 1515 bp, Homo sapiens
  misc_feature: Y box binding protein 1 (YBX1)
cgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacctaaagacgtctctttttggtaataacaaacgtgttttttaaaaaagcctggtttttctcaatacgcctttaaaggtttttaaattgtttcatatctggtcaagttgagatttttaagaacttcatttttaatttgtaataaaagtttacaacttgattttttcaaaaaagtcaacaaactgcaagcacctgttaataaaggtcttaaataataaaaaaaaaaaaaaa

SEQ ID NO: 16 - protein, 324 aa, Homo sapiens
  misc_feature: Y box binding protein 1 (YBX1)
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 17 - DNA, 488 bp, Homo sapiens
  misc_feature: ribosomal protein S17 (RPS17)
gtttcctcttttaccaaggacccgccaacatgggccgcgttcgcaccaaaaccgtgaagaaggcggcccgggtcatcatagaaaagtactacacgcgcctgggcaacgacttccacacgaacaagcgcgtgtgcgaggagatcgccattatccccagcaaaaagctccgcaacaagatagcaggttatgtcacgcatctgatgaagcgaattcagagaggcccagtaagaggtatctccatcaagctgcaggaggaggagagagaaaggagagacaattatgttcctgaggtctcagccttggatcaggagattattgaagtagatcctgacactaaggaaatgctgaagcttttggacttcggcagtctgtccaaccttcaggtcactcagcctacagttgggatgaatttcaaaacgcctcggggacctgtttgaattttttctgtagtgctgtattattttcaataaatctgggacaacagcctt

SEQ ID NO: 18 - protein, 135 aa, Homo sapiens [sequence omitted]

SEQ ID NO: 19 - DNA, 386 bp, Homo sapiens
  misc_feature: ribosomal protein S29 (RPS29)
ctcaaaaatttgaagagcgcatgcgtgggccagcttcttccttttacctcgttgcactgctgagagcaagatgggtcaccagcagctgtactggagccacccgcgaaaattcggccagggttctcgctcttgtcgtgtctgttcaaaccggcacggtctgatccggaaatatggcctcaatatgtgccgccagtgtttccgtcagtacgcgaaggatatcggtttcattaagttggactaaatgctcttccttcagaggattatccggggcatctactcaatgaaaaaccatgataattctttgtatataaaataaacatttgaaaaaacccttcaaaaaaaaaaaaaaaaaaaaaaaaaaaaaaaaaaaaaaaaaaaaaaaaaaa

SEQ ID NO: 20 - protein, 56 aa, Homo sapiens [sequence omitted]

SEQ ID NO: 21 - DNA, 827 bp, Homo sapiens
  misc_feature: pancreatic progenitor cell differentiation and proliferation factor (PPDPF)
accccgcgcgcgcctctctgtcgtggcgcggcttcccgcggtcttctctgcaaatgggctccgtggcctagcgcccccgtccccgccacccgtgatcgtgcgccgaggcccgcgaggggtcgccgcccagatcccaccagccagcaagctaaagcatggcggccatcccctccagcggctcgctcgtggccacccacgactactaccggcgccgcctgggttccacttccagcaacagctcctgcagcagtaccgagtgccccggggaagccattccccaccccccaggtctccccaaggctgacccgggtcattggtgggccagcttctttttcgggaagtccaccctcccgttcatggccacggtgttggagtccgcagagcactcggaacctccccaggcctccagcagcatgaccgcctgtggcctggctcgggacgccccgaggaagcagcccggcggtcagtccagcacagccagcgctgggcccccgtcctgacctgagcggttaccaccagccccaggcctgcggaggcgctagtccaccagagcccctccccgcccctctccccactccgcatccctcgcccccctccccacctcccaccccccaccctgtaaactaggcggctgcagcaagcagaccttcgcatcaacacagcagacaccaaaaaccagtgagagccccgctctctaccgcccggccccagcactcgctagctttcctgacacctggaactgtgcacctggcaccaagcggaaaataaactccaagcagccagtagccccgatggtgtgtgcctgagctgtgtggcccgaggttcca

SEQ ID NO: 22 - protein, 114 aa, Homo sapiens
  misc_feature: pancreatic progenitor cell differentiation and proliferation factor (PPDPF)
MAAIPSSGSLVATHDYYRRRLGSTSSNSSCSSTECPGEAIPHPPGLPKADPGHWWASFFFGKSTLPFMATVLESAEHSEPPQASSSMTACGLARDAPRKQPGGQSSTASAGPPS

SEQ ID NO: 23 - DNA, 1931 bp, Homo sapiens
  misc_feature: Y-box binding protein 3 (YBX3)
agtaagatcgagcgaggagcccaagagagagcgcgcagcacgaagctcgagccgcctccgccgcgcgaccccacctcggccgccgccgcctgcgccgcgagatccgccccggcctccccgagagcgagccccggccgccgcgaccaccagccgcgctaaccgccgaccaaccgccaccgaggcgcctgagcgagagcagaggaggaggaggcatgagtgaggcgggcgaggccaccaccaccaccaccaccaccctcccgcaggctccgacggaggcggccgccgcggctccccaggaccccgcgcccaagagcccggtgggcagcggtgcgccccaggccgcggccccggcgcccgccgcccacgtcgcaggaaaccccggtggggacgcggcccccgcagccacgggcaccgcggccgccgcctctttagccaccgccgccggcagcgaagacgcggagaaaaaagttctcgccaccaaagtccttggcactgtcaaatggttcaacgtcagaaatggatatggatttataaatcgaaatgacaccaaagaagatgtatttgtacatcagactgccatcaagaagaataacccacggaaatatctgcgcagtgtaggagatggagaaactgtagagtttgatgtggttgaaggagagaagggtgcagaagctgccaatgtgactggcccggatggagttcctgtggaagggagtcgttacgctgcagatcggcgccgttacagacgtggctactatggaaggcgccgtggccctccccggaattacgctggggaggaggaggaggaagggagcggcagcagtgaaggatttgacccccctgccactgataggcagttctctggggcccggaatcagctgcgccgcccccagtatcgccctcagtaccggcagcggcggttcccgccttaccacgtgggacagacctttgaccgtcgctcacgggtcttaccccatcccaacagaatacaggctggtgagattggagagatgaaggatggagtcccagagggagcacaacttcagggaccggttcatcgaaatccaacttaccgcccaaggtaccgtagcaggggacctcctcgcccacgacctgccccagcagttggagaggctgaagataaagaaaatcagcaagccaccagtggtccaaaccagccgtctgttcgccgtggataccggcgtccctacaattaccggcgtcgcccgcgtcctcctaacgctccttcacaagatggcaaagaggccaaggcaggtgaagcaccaactgagaaccctgctccacccacccagcagagcagtgctgagtaacaccaggctcctcaggcaccttcaccatcggcaggtgacctaaagaattaatgaccattcagaaataaagcaaaaagcaggccacaaccttaaccaacaccaaagaaacatccaagcaataaagtggaagactaaccaagatttggacattggaatgtttactgttattctttaagaaacaactacaaaaagaaaatgtcaacaaatttttccagcaagctgagaacctgggaattcctgcacggaagacaagagagtagcctctccagtttcagcaaccgctaggtttctattttttttcctggtttttactgttttggtaatatatatattgaaacaagaaatattaataccacatggggagaaccccaaccaaagaaatctgaaatatatagtaaatgcttttttttccgtttttgttcattttggatgctggtgctaaacctccaagtgtcatgatttaaaaaaaaaaaaaatttatgtccttcttatttatttctaggatgaggggaggataacatttttgctttcttatgtgactctctttgaaaatgtgcagtaagaaattcctcaaaaataaaatttttacccttcagagga

SEQ ID NO: 24 - protein, 372 aa, Homo sapiens
  misc_feature: Y-box binding protein 3 (YBX3)
MSEAGEATTTTTTTLPQAPTEAAAAAPQDPAPKSPVGSGAPQAAAPAPAAHVAGNPGGDAAPAATGTAAAASLATAAGSEDAEKKVLATKVLGTVKWFNVRNGYGFINRNDTKEDVFVHQTAIKKNNPRKYLRSVGDGETVEFDVVEGEKGAEAANVTGPDGVPVEGSRYAADRRRYRRGYYGRRRGPPRNYAGEEEEEGSGSSEGFDPPATDRQFSGARNQLRRPQYRPQYRQRRFPPYHVGQTFDRRSRVLPHPNRIQAGEIGEMKDGVPEGAQLQGPVHRNPTYRPRYRSRGPPRPRPAPAVGEAEDKENQQATSGPNQPSVRRGYRRPYNYRRRPRPPNAPSQDGKEAKAGEAPTENPAPPTQQSSAE

SEQ ID NO: 25 - DNA, 2154 bp, Homo sapiens
  misc_feature: vimentin (VIM)
gggaggcccacgtatggcgcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactc

SEQ ID NO: 26 - protein, 466 aa, Homo sapiens
  misc_feature: vimentin (VIM)
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 27 - DNA, 317 bp, Homo sapiens
  misc_feature: NADHubiquinone oxidoreductase subunit B1 (NDUFB1)
actggcgcgggttgagttccctgttgcccttggtctcggggtcgctgtaggcgctgaggctgcagctatcatggtgaacttacttcagattgtgcgggaccactgggttcatgttcttgtccctatgggatttgtcattggatgttatttagacagaaagagtgatgaacggctaactgccttccggaacaagagtatgttatttaaaagggaattgcaacccagtgaagaagttacctggaagtaaagactggctagattatcgaatgttcacattttaaagttctgagagaaataaaaacatgaagaatctgaaa

SEQ ID NO: 28 - protein, 58 aa, Homo sapiens
  misc_feature: NADHubiquinone oxidoreductase subunit B1 (NDUFB1)
MVNLLQIVRDHWVHVLVPMGFVIGCYLDRKSDERLTAFRNKSMLFKRELQPSEEVTWK

SEQ ID NO: 29 - DNA, 644 bp, Homo sapiens
  misc_feature: ribosomal protein L34 (RPL34)
gttgtctgcaggtatggatgttgttctcttttccctgtctttatttccttaccaatcggctgccatccgaggagctgaggaagcctagagctctcagaagcagtcctttgagctggtgtaggggcactcagaatggtccagcgtttgacataccgacgtaggctttcctacaatacagcctctaacaaaactaggctgtcccgaacccctggtaatagaattgtttacctttataccaagaaggttgggaaagcaccaaaatctgcatgtggtgtgtgcccaggcagacttcgaggggttcgtgctgtaagacctaaagttcttatgagattgtccaaaacaaagaaacatgtcagcagggcctatggtggttccatgtgtgctaaatgtgttcgtgacaggatcaagcgtgctttccttatcgaggagcagaaaatcgttgtgaaagtgttgaaggcacaagcacagagtcagaaagctaaataaaaaaatgaaacttttttgagtaataaaaatgaaaagacgctgtatgtatgactttttttttttctgttgtaatgtgttagtatacagattttgtttctgtatggtatttggggaccctatagtttttagcagattactttttcttgttttgtttgttg

SEQ ID NO: 30 - protein, 117 aa, Homo sapiens [sequence omitted]

SEQ ID NO: 31 - DNA, 557 bp, Homo sapiens
  misc_feature: macrophage migration inhibitory factor (MIF)
agtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagacta

SEQ ID NO: 32 - protein, 115 aa, Homo sapiens
  misc_feature: macrophage migration inhibitory factor (MIF)
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 33 - DNA, 1889 bp, Homo sapiens
  misc_feature: phospholipid transfer protein (PLTP)
gccagaccccgtcgcccggatcccctgagctgcccgccatcccacgtgaccgcgccgccccccagctccaccgctgagcccgctcgccatggccctcttcggggccctcttcctagcgctgctggcaggcgcacatgcagagttcccaggctgcaagatccgcgtcacctccaaggcgctggagctggtgaagcaggaggggctgcgctttctggagcaagagctggagactatcaccattccggacctgcggggcaaagaaggccacttctactacaacatctctgaggtgaaggtcacagagctgcaactgacatcttccgagctcgatttccagccacagcaggagctgatgcttcaaatcaccaatgcctccttggggctgcgcttccggagacagctgctctactggttcttctatgatgggggctacatcaacgcctcagctgagggtgtgtccatccgcactggtctggagctctcccgggatcccgctggacggatgaaagtgtccaatgtctcctgccaggcctctgtctccagaatgcacgcggccttcgggggaaccttcaagaaggtgtatgattttctctccacgttcatcacctcagggatgcgcttcctcctcaaccagcagatctgccctgtcctctaccacgcagggacggtcctgctcaactccctcctggacaccgtgcctgtgcgcagttctgtggacgagcttgttggcattgactattccctcatgaaggatcctgtggcttccaccagcaacctggacatggacttccggggggccttcttccccctgactgagaggaactggagcctccccaaccgggcagtggagccccagctgcaggaggaagagcggatggtgtatgtggccttctctgagttcttcttcgactctgccatggagagctacttccgggcgggggccctgcagctgttgctggtgggggacaaggtgccccacgacctggacatgctgctgagggccacctactttgggagcattgtcctgctgagcccagcagtgattgactccccattgaagctggagctgcgggtcctggccccaccgcgctgcaccatcaagccctctggcaccaccatctctgtcactgctagcgtcaccattgccctggtcccaccagaccagcctgaggtccagctgtccagcatgactatggacgcccgtctcagcgccaagatggctctccgggggaaggccctgcgcacgcagctggacctgcgcaggttccgaatctattccaaccattctgcactggagtcgctggctctgatcccattacaggcccctctgaagaccatgctgcagattggggtgatgcccatgctcaatgagcggacctggcgtggggtgcagatcccactacctgagggcatcaactttgtgcatgaggtggtgacgaaccatgcgggattcctcaccatcggggctgatctccactttgccaaagggctgcgagaggtgattgagaagaaccggcctgctgatgtcagggcgtccactgcccccacaccgtccacagcagctgtctgagccctcaatccccaagctggcagctgtcattcaggaccccaacccctctcagcccctcttttcccacattcatagcctgtagtgccccctctaacccccagtgccacagagaagacgggatttgaagctgtacccaatttaattccataatcaatctatcaattacagtccgtccaccacctccctgtgggctgtcctgagctctgttgggttcctgggatggaatcagtgcatcataaagggcattctttaagcagagaaggggccaggccaccccattcaggaactgctgcgggaataaagtgctaacttgccccca

SEQ ID NO: 34 - protein, 493 aa, Homo sapiens
  misc_feature: phospholipid transfer protein (PLTP)
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 35 - DNA, 1145 bp, Homo sapiens
  misc_feature: ribosomal protein L38 (RPL38)
ctttttcgtccttttccccggttgctgcttgctgtgagtgtctctagggtgatacgtgggtgagaaaggtcctggtccgcgccagagcccagcgcgcctcgtcgccatgcctcggaaaattgaggaaatcaaggacttcctgctcacagcccgacgaaaggatgccaaatctgtcaagatcaagaaaaataaggacaacgtgaagtttaaagttcgatgcagcagatacctttacaccctggtcatcactgacaaagagaaggcagagaaactgaagcagtccctgccccccggtttggcagtgaaggaactgaaatgaaccagacacactgattggaactgtattatattaaaatactaaaaatcctaagtgtctttcgtctttgcggatgggaaagggaaaaatgctacctcgtagtggcttctgatgggaacaggacgcgggttctgttgctgccttcctgtgtctttttttttttttttttttctttctttgagacggagtcttgctctgtggctcatcctggagcacagtggtgcgatatcagctcactaccacctccgcctcctgggttcaagcgactgtcctgcctcagcctcccgagtggctgggattacaggcacacatcaccacgcctggccaatttttgtatttgtagtagagacagggtttcactgcctgcctcagcctcccatagtgctgggattacaggcatgagcctccgtgcccggtgcatccctaatcttgagcatgatctcagtcggcaaatgaggccatctgttttcagcctgtttgaaaataagatgtggggaggccatgatggaaatagcacgtggggttaaacataactggcagatgtgggagcgatggtggggcatgccattcaaacaggtcccaaaatgggtgcaacaaggtatagcacatctaccactcgctaacttgactgacttggagaaatgactacacttttgcctgtttcctcagttggaaaatagccatattaacacctctttcattggcttgctgtcagggtactgggatggggggaggtgcatgggttggggtggccaccaggtggtgctgtgccacagcgggcagcccctctggaaatgactggcatcataaaatctgtcttcatacccga

SEQ ID NO: 36 - protein, 70 aa, Homo sapiens [sequence omitted]

SEQ ID NO: 37 - DNA, 358 bp, Homo sapiens
  misc_feature: ribosomal protein S21 (RPS21)
cctttctctctcgcgcgcggtgtggtggcagcaggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaaccta

SEQ ID NO: 38 - protein, 83 aa, Homo sapiens
  misc_feature: ribosomal protein S21 (RPS21)
MQNDAGEFVDLYVPRKCSASNRIIGAKDHASIQMNVAEVDKVTGRFNGQFKTYAICGAIRRMGESDDSILRLAKADGIVSKNF

SEQ ID NO: 39 - DNA, 588 bp, Homo sapiens
  misc_feature: ribosomal protein S10 (RPS10)
cctttccagccccggtaccggaccctgcagccgcagagatgttgatgcctaagaagaaccggattgccatttatgaactcctttttaaggagggagtcatggtggccaagaaggatgtccacatgcctaagcacccggagctggcagacaagaatgtgcccaaccttcatgtcatgaaggccatgcagtctctcaagtcccgaggctacgtgaaggaacagtttgcctggagacatttctactggtaccttaccaatgagggtatccagtatctccgtgattaccttcatctgcccccggagattgtgcctgccaccctacgccgtagccgtccagagactggcaggcctcggcctaaaggtctggagggtgagcgacctgcgagactcacaagaggggaagctgacagagatacctacagacggagtgctgtgccacctggtgccgacaagaaagccgaggctggggctgggtcagcaaccgaattccagtttagaggcggatttggtcgtggacgtggtcagccacctcagtaaaattggagaggattcttttgcattgaataaacttacagccaaaaaaccttaa

SEQ ID NO: 40 - protein, 165 aa, Homo sapiens
  misc_feature: ribosomal protein S10 (RPS10)
MLMPKKNRIAIYELLFKEGVMVAKKDVHMPKHPELADKNVPNLHVMKAMQSLKSRGYVKEQFAWRHFYWYLTNEGIQYLRDYLHLPPEIVPATLRRSRPETGRPRPKGLEGERPARLTRGEADRDTYRRSAVPPGADKKAEAGAGSATEFQFRGGFGRGRGQPPQ

SEQ ID NO: 41 - DNA, 914 bp, Homo sapiens
  misc_feature: ribonuclease A family member 1, pancreatic (RNASE1)
gcagaaactggccttccatctctctcagacaccaagctgcagatccaggcttttctgggaaagtgaggccaccatggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacctaaggtcagagcagcgagataccccacctccctcaacctcatcctctccacagctgcctcttccctcttccttccctgctgtgaaagaagtaactacagttagggctcctattcaacacacacatgcttccctttcctgagtcccatccctgcgtgattttgggggtgaagagtgggttgtgaggtgggccccatgttaacccctccactctttctttcaataaaacgcagttgcaaacacctgatttctgaagcggttctgtctaggtactgtttctggcattgccttccagcaaggggtaagaactgtaaatctgattcactttggagaacggtgaatggagtaattaaatgccttcccttctgacttgga

SEQ ID NO: 42 - protein, 156 aa, Homo sapiens
  misc_feature: ribonuclease A family member 1, pancreatic (RNASE1)
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 43 - DNA, 483 bp, Homo sapiens
  misc_feature: ribosomal protein S25 (RPS25)
ctttttgtccgacatcttgacgaggctgcggtgtctgctgctattctccgagcttcgcaatgccgcctaaggacgacaagaagaagaaggacgctggaaagtcggccaagaaagacaaagacccagtgaacaaatccgggggcaaggccaaaaagaagaagtggtccaaaggcaaagttcgggacaagctcaataacttagtcttgtttgacaaagctacctatgataaactctgtaaggaagttcccaactataaacttataaccccagctgtggtctctgagagactgaagattcgaggctccctggccagggcagcccttcaggagctccttagtaaaggacttatcaaactggtttcaaagcacagagctcaagtaatttacaccagaaataccaagggtggagatgctccagctgctggtgaagatgcatgaataggtccaaccagctgtacatttggaaaaataaaactttattaaa

SEQ ID NO: 44 - protein, 125 aa, Homo sapiens [sequence omitted]

SEQ ID NO: 45 - DNA, 528 bp, Homo sapiens
  misc_feature: 60S ribosomal protein L26 (RPL26)
ctcttcccttttgcggccatcaccgaagcgggagcggccaaaatgaagtttaatccctttgtgacttccgaccgaagcaagaatcgcaaaaggcatttcaatgcaccttcccacattcgaaggaagattatgtcttcccctctttccaaagagctgagacagaagtacaacgtgcgatccatgcccatccgaaaggatgatgaagttcaggttgtacgtggacactataaaggtcagcaaattggcaaagtagtccaggtttacaggaagaaatatgttatctacattgaacgggtgcagcgggaaaaggctaatggcacaactgtccacgtaggcattcaccccagcaaggtggttatcactaggctaaaactggacaaagaccgcaaaaagatcctcgaacggaaagccaaatctcgccaagtaggaaaggaaaagggcaaatacaaggaagaaaccattgagaagatgcaggaataaagtaatcttatatacaagctttgattaaaacttgaaacaaagagcctg

SEQ ID NO: 46 - protein, 145 aa, Homo sapiens [sequence omitted]

SEQ ID NO: 47 - DNA, 2992 bp, Homo sapiens
  misc_feature: ribosomal protein L37a (RPL37A)
ctttctgggctcggacctaggtcgcggcgacatggccaaacgtaccaagaaagtcgggatcgtcggtaaatacgggacccgctatggggcctccctccggaaaatggtgaagaaaattgaaatcagccagcacgccaagtacacttgctctttctgtggcaaaaccaagatgaagagacgagctgtggggatctggcactgtggttcctgcatgaagacagtggctggcggtgcctggacgtacaataccacttccgctgtcacggtaaagtccgccatcagaagactgaaggagttgaaagaccagtagacgctcctctactctttgagacatcactggcctataataaatgggttaatttatgtaacaaaattgccttggcttgttaactttattagacattctgatgtttgcattgtgtaaatactgttgtattggaaaagcatgccaagatggattattgtaattcagtgtcttttttagtagtcaaatggtaaaatgcagcataagaatataagtcttccaagttagatatgagtgttagctttttataagtctgctcctgccagtttgactttgagatacattggagccaactgtaaactttagtttttaaattacagttagtttttttgtttgtttttgaggcggagtctctgttacccaggctggagtgcagtataccagtcttggcccacttcaacctccacttcttaggttcaagcgattctcctgcctcagcctcctgagtagctgggattacaggcacgcaccaccatacctggctaatttttgtattttgagtagagatggagttttcaccacattggccaggctgttcttgaactgacctcaagcgatccacctgccttggccttccagagtgctgggattacaggtgtgagccaccacgcccagccttgcatttaatatttttataatgtgtctaggctgggtgcggtgactcacgcctgaaatcccagcactttgggtggctgaggcaggtggattacttgaggccaggagattgagaccagtgtggccaacatagcaaaaacccgtctcgacgaaaaatacaaaaaatagcttggtatggtggcacatgcctgtaatcccagctactttggaggctcaggcacaagaatcacttgaacctacgaggcggaggttgcagtgggccaggatcgtgccactgcactttatttagccaggacaacactctgtctccaaaaaaaagtttctgaaggtaaaagatatactaaaggatatacatatactgcctcttctatgatgtttcatttgacctacaccatcagcatcttcaggaaacagtatgaaactaatttcacattaaatgttgcaaacgtctgcaattctccagtttttagtttgcacgtaaggattcgtaggattgatttcagattgacagatggttgtagtaacaaaaaaatgaattgccttggagaattcgagttattttaggcctgcctttgacacttttatgcacttaattcactgccaatttagtctaactcctcaaccatctgtggtatggacttctcagtcctcctaattggtatgtcttttatctatcccagagtcagtagtcctttaaggatgcaaatttaattcgataagaatgtggatttacttcatctttaggacagagtaaaaacctttatttcacttggtctgacccctatcttctataacttgtttttttgctcattactggcctttgcacggatgtctcccttcacctgtacctcctgcattgcctctggcagaaacatcgctaccccaggagaatacttcactgggtcaggcatttgctagatccatagcagtaactcatctgtggatctcatttgtgtcacaaaatggtactcctactgattttgtaatgtgaagcaagtactgtgggtcagtaggggtaggaccaggagtgacaagtcaggattttcaggcttcataggaatgacacagctctccaggtgaatctcctggagaattctccataggatctccaggtgagtgaccaggatttcatgtaagcatagggaattgaatgagcattagatggtctaatgctgcggttaagaacaccaacttagccaaactgcctagatagttgggtgacttgggttaagtgacttaacctctctgcttcagtttcctgtataattcttggtagtagaatggagtatatgctgggtgtttggattattaatttggagtcttacaaagctgctatgttacatgattaggcatggaaaacttcggtttttataatcagtgaagtactgctttttgtttgttttctgagagagtgtctgtcacccaggctgaagtgcagtggcatgatctcagttcactgcagcctctacctcccgggttcaagcagttctcgtgcctcagcctccgaagtagctgggattacaggcttatgccacaaagctcagctagtttttgtattttttatttttagtagagatgggctttcgccatgttgaccaggctggtctcaaactcccaggctcaactgataacgtctgccttggcctcccaaagtgctgggattctgtaagccacctcacccagccatgtattgcttttttatgacagttaaaagaggaaagggcgggggacagtttacagaccagtgaaagtgctgagataaatatttttatacctaaggaaatagtgattttcccaagatgatagggaggcaggtgatgtagatagggctgaagattagatttctggctcctatctgtgattttctgctgtaccagtctgccttctattttatgggctcagtattccttacctgcctcttcccatgctaaagatggcccaccttcgttttgttatttaagcaacttcatcccctggcttgttttcacagtggttttctgagcctttgactctaagtcatctaattgaacattgtgttgtgatataaaaagtaagttaggcttgtgtttttca

SEQ ID NO: 48 - protein, 92 aa, Homo sapiens [sequence omitted]

SEQ ID NO: 49 - DNA, 2137 bp, Homo sapiens
  misc_feature: 40S ribosomal protein S15a (RPS15A) Variant 1
ctctttccgccatctttccgcgccgccacaatggtgcgcatgaatgtcctggcagatgctctcaagagtatcaacaatgccgaaaagagaggcaaacgccaggtgcttattaggccgtgctccaaagtcatcgtccggtttctcactgtgatgatgaagcatggttacattggcgaatttgaaatcattgatgaccacagagctgggaaaattgttgtgaacctcacaggcaggctaaacaagtgtggggtgatcagccccagatttgacgtgcaactcaaagacctggaaaaatggcagaataatctgcttccatcccgccagtttggtttcattgtactgacaacctcagctggcatcatggaccatgaagaagcaagacgaaaacacacaggagggaaaatcctgggattctttttctagggatgtaatacatatatttacaaataaaatgcctcatggactctggtgcttccacttggtcgttttgagcctttacagcagtgtagccacagcttctgcggcagcatgcagttgcttcgtttatcggtgaatgcgattccctgaagtgactaatacagccaagggaaaaagttcttatgaaaccagtatgcctaagaaacagtcacccctgctgtctgccaaaaccaggtatttgacactaaatattttagttgtatttcagtttttttttttttttttcttttttggagacagagtctgactctattgctcaggctggagtgcagtggcgcgatcttggcccactgcaacctccacctcccgggttcaagtgattgtcctgtctcagccgcctgagtaactgggattacaagtgtgtgtcgccacatccggctaatttttatattttagtagagacagggtttcgccatgttgcccaggctggtcttgagttctgggcctcaagtgatcagcctacctcggcctcccgaagtgctgggattacagccacgagccattacacctggcctatatttcagtattttctattagtttttgatgaatttgttttgcctggctaggattattctgtagataggattttagatctggcttttgtcactgactgctgtaataaatacttgctaggaatttttttttttttttttttttttttaagacaaagtcgctctgtcacacaggctggagtgcagtggcatgatcttggctcactgcacctccgcttcccagattcaagtagttctcgtgcctcagcctcctgagtagctgggattacaggtgtgtgccaccatgtctggctgatttttgtatttttagtagaggtggggtttcaccatgttggccaggctggtctcgaactcctgatctcaagtgatctgcctgccttggtctcccaaagtgctgggattacaggtgtgaccaccacgccctgccttaagaattgttccaagagaatctggtgccacttgcaggtgcccattgaagtgcaatgggcactgttgatcactgaggaggtagtgggtgctgacccggtgctggggcctgtcccctagtctctgctttgcccttggctagctaggtggtgtgccaagtggggagagaagctaccttattaaggggcatggatcagcttcctgaaaggagggcctgcctctgtaagatatgggaagtcgctgagaatgttacagaacggccctagagatggggcagataacggcccccatttgtgagaagtgagttgggaggcatgtttggggcctctgatgtttgggaggctgtgggtaattaacatgagttttggggtccagcagcagaattcaggtttcctcttccactcagtaacctcagcatccgtatctgtaatgggaatgatacaaaacctatccccaagttgagggaaaaatgagattgtgtaaagcgcacttggcacatgacagtcacaagcatggggacagtgagtccagaaggattttcttatgccagcattgtaagccctaggatcacagggctctggcttgtttaaccatcgtgtctctggtccctagcctgcaaacctggtgtgtagggatgcctcagtcgcttacatgttgattgagtgaatcgtcggtttctttctggacactgacttcaaaaataaaataggatatgaaaatgg

SEQ ID NO: 50 - protein, 130 aa, Homo sapiens [sequence omitted]

SEQ ID NO: 51 - DNA, 1773 bp, Homo sapiens
  misc_feature: eukaryotic translation elongation factor 1 alpha 2 (EEF1A2)
ccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggtgccgcgctccgaaccccgggcccgggcccccgccccgcccccgccccgcgcgccggtccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgacccca

SEQ ID NO: 52 - protein, 463 aa, Homo sapiens
  misc_feature: eukaryotic translation elongation factor 1 alpha 2 (EEF1A2)
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 53 - DNA, 352 bp, Homo sapiens
  misc_feature: ribosomal protein S27 (RPS27)
cctttccggcggtgacgacctacgcacacgagaacatgcctctcgcaaaggatctccttcatccctctccagaagaggagaagaggaaacacaagaagaaacgcctggtgcagagccccaattcctacttcatggatgtgaaatgcccaggatgctataaaatcaccacggtctttagccatgcacaaacggtagttttgtgtgttggctgctccactgtcctctgccagcctacaggaggaaaagcaaggcttacagaaggatgttccttcaggaggaagcagcactaaaagcactctgagtcaagatgagtgggaaaccatctcaataaacacattttggataaatcctg

SEQ ID NO: 54 - protein, 84 aa, Homo sapiens
  misc_feature: ribosomal protein S27 (RPS27)
MPLAKDLLHPSPEEEKRKHKKKRLVQSPNSYFMDVKCPGCYKITTVFSHAQTVVLCVGCSTVLCQPTGGKARLTEGCSFRRKQH

SEQ ID NO: 55 - DNA, 3405 bp, Homo sapiens
  misc_feature: nexilin F-actin binding protein (NEXN), transcript variant 1
aacagctgcagccggcgctgggcccgcctggaatgcgggaacaggctgcacaccaggacttttatggaaacttgctgctggagacggcggcggcggcggcggcagcggcagccagaggactcccagcggctggagcagaagtgttagcggccagagctcccagacccctacccacagccaggcgggacgcgcacagtccctccacgcggaaagaagtaccttcgccggtcaccggctcctgcagggtgcaaatatatacagagcttcataatcagcccaagaccacatagagcaaacatgaatgatatttcccaaaaggctgagattctgctttcttcatctaaacctgtcccaaaaacctatgtaccaaaacttggcaagggtgatgtaaaggataagtttgaagccatgcagagagccagggaagaaagaaatcaaaggagatctagagacgaaaaacaaagaagaaaagaacaatatattagagagagagaatggaacaggagaaagcaggagattaaagaaatgcttgcttctgatgatgaggaagatgtatcttctaaagtagaaaaggcttatgttccaaaattaacaggaactgtgaagggtagatttgctgaaatggagaaacaaagacaagaggaacaaaggaagagaacggaggaggaacgaaaacgcagaattgagcaggatatgttagaaaagaggaaaatacagcgtgaattagcaaaaagggctgaacagattgaggacataaacaatacgggaactgaatcagcatcagaggaaggagatgattcactacttataactgtggtacctgtcaaatcatataaaacatctggaaaaatgaaaaagaattttgaggatctagaaaaagaacgtgaagagaaagaaaggatcaagtacgaggaagataaaagaataagatatgaagaacaacgaccatctctcaaggaagcaaagtgtctttcattagttatggatgatgaaatagaaagtgaagcaaaaaaagaatcactttctcccggaaaattgaaactaacttttgaagaactggagcgacaaagacaagaaaaccgaaagaagcaagctgaagaggaagcaagaaaacgtttagaagaagagaagcgtgcttttgaagaagcaaggcggcaaatggtaaatgaagatgaggaaaaccaagacacagcaaaaatttttaaagggtaccgccctggtaaactcaaactcagttttgaagaaatggaaaggcaaagaagagaagatgaaaaaaggaaagcagaagaagaagccagaaggagaatagaggaagaaaagaaggcgtttgctgaagcaaggagaaatatggtagtagatgatgactccccagagatgtataagacaatctctcaagaatttcttacaccgggaaaactggaaattaattttgaagaattattaaaacaaaaaatggaagaagaaaaacgacgaacagaggaggaacggaagcataagctagaaatggagaaacaagaatttgaacaactgagacaggaaatgggagaggaagaggaagaaaatgaaacctttggattgagcagagaatatgaagaactgatcaaattaaaaaggagtggctctattcaagctaaaaacctaaaaagcaagtttgaaaaaattggacagttgtctgaaaaagaaatacagaaaaaaatagaagaagagcgagcaagaaggagagcaattgaccttgaaattaaagagcgagaagctgaaaattttcatgaggaagatgatgttgatgttaggcctgcaagaaaaagcgaggctccatttactcacaaagtgaatatgaaagctagatttgaacaaatggctaaggcaagagaagaagaagaacaaagaagaattgaagaacaaaagttactacgcatgcagtttgaacaaagggaaattgatgcagcactacaaaagaaaagagaagaggaggaggaggaagaaggtagcatcatgaatggctccactgctgaagatgaagagcaaaccagatcaggagctccatggttcaagaagcctcttaaaaacacatcagttgtagacagtgagccagtcagatttacggttaaagtaacaggagaacccaaaccagaaattacatggtggtttgaaggagaaatactgcaggatggagaagactatcaatatattgaaaggggagaaacttactgcctttacttaccagaaactttcccagaagatggaggagagtatatgtgtaaagcagtcaacaataaaggatctgcagctagtacctgtattcttaccattgaaagtaagaattaatcactctttttatcttttattctattaatttttttttccttaaaatcacttttcttcttctcttttttagctgatgactactagctcccctcccctctccctggaactttctctttcactccaactttcttactacatccatcttttctgtggcggggccaaaaaaggaaaccaggagtgccactatgctgacttcttattccttttcataacagtcttcaaagcacagctcatctaaagaatgcctacttcttttccaaataagcatcagatttatcgcctattatgcagtaacagtcaataaaatgtacttatgggggggaattactcaattattctatcagaacctattataaagactgtatttcccatagacgtttacagcaactatgtttaaaaaacaaaaacaaaaaaaaaacacacaaacctaagtagaatacattattttgcatgaaggaatgtcatttctgagctttttacacctaaaattaggctgaaatagctgagataattaatttggaacctatcaatttgagtggactttttctttagtagtacaccattttggttgttgtagtttcaaagtctttctgaagcagatatattgggattggagcggggtggggaaaactgtcactcctttcagaggaaaaggggaggagcatggagaaaaacaaaaattaaaggacttaaagaatggctatacagtgttgagtgttgaggatattaaacatgttatttttcaaacgtatgtaatatatattaaatttataaagcaaatttatgttgtgatcttgcctgaacaaattatattttaatgaaaaaactttctattaatagttcacgcaagagaaaacactttcaacatagtcgaaggcttcaagatctaagtgtatcagacttagggaaaaagtggcacaaccttcgatttaaaattctagtctttaaaatgagtttgtaaataattagctattacgttctattaagttgttttatattttaattttctggaagacaattttattttacaacgtgaacccaaataaagtaacttctgtatttaaaaaaaaaaaaaaaaaa

SEQ ID NO: 56 - protein, 674 aa, Homo sapiens
  misc_feature: nexilin F-actin binding protein (NEXN), transcript variant 1
MNDISQKAEILLSSSKPVPKTYVPKLGKGDVKDKFEAMQRAREERNQRRSRDEKQRRKEQYIREREWNRRKQEIKEMLASDDEEDVSSKVEKAYVPKLTGTVKGRFAEMEKQRQEEQRKRTEEERKRRIEQDMLEKRKIQRELAKRAEQIEDINNTGTESASEEGDDSLLITVVPVKSYKTSGKMKKNFEDLEKEREEKERIKYEEDKRIRYEEQRPSLKEAKCLSLVMDDEIESEAKKESLSPGKLKLTFEELERQRQENRKKQAEEEARKRLEEEKRAFEEARRQMVNEDEENQDTAKIFKGYRPGKLKLSFEEMERQRREDEKRKAEEEARRRIEEEKKAFAEARRNMVVDDDSPEMYKTISQEFLTPGKLEINFEELLKQKMEEEKRRTEEERKHKLEMEKQEFEQLRQEMGEEEEENETFGLSREYEELIKLKRSGSIQAKNLKSKFEKIGQLSKEIQKKIEEERARRRAIDLEIKEREAENFHEEDDVDVRPARKSEAPFTHKVNMKARFEQMAKAREEEEQRRIEEQKLLRMQFEQREIDAALQKKREEEEEEEGSIMNGSTAEDEEQTRSGAPWFKKPLKNTSVVDSEPVRFTVKVTGEPKPEITWWFEGEILQDGEDYQYIERGETYCLYLPETFPEDGGEYMCKAVNNKGSAASTCILTIESKN

SEQ ID NO: 57 - DNA, 5914 bp, Homo sapiens
  misc_feature: collagen type I alpha 1 chain (COL1A1)
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 58 - protein, 1464 aa, Homo sapiens
  misc_feature: collagen type I alpha 1 chain (COL1A1)
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 59 - DNA, 2706 bp, Homo sapiens
  misc_feature: ribosomal protein L23 (RPL23)
cggcgttcaagatgtcgaagcgaggacgtggtgggtcctctggtgcgaaattccggatttccttgggtcttccggtaggagctgtaatcaattgtgctgacaacacaggagccaaaaacctgtatatcatctccgtgaaggggatcaagggacggctgaacagacttcccgctgctggtgtgggtgacatggtgatggccacagtcaagaaaggcaaaccagagctcagaaaaaaggtacatccagcagtggtcattcgacaacgaaagtcataccgtagaaaagatggcgtgtttctttattttgaagataatgcaggagtcatagtgaacaataaaggcgagatgaaaggttctgccattacaggaccagtagcaaaggagtgtgcagacttgtggccccggattgcatccaatgctggcagcattgcatgattctccagtatatttgtaaaaaataaaaaaaaaaactaaacccattaaaaagtatttgtttgcagtgcttgtctccctgttcacttccagggttcaagcgattctcctgcctcagcctcctaagtagctgggattacaggcacctgccgtcatgcctggctagtttttgtatttttggtggagacagggtttcaccatgtttgccaaacgactcaaactcctgatctcaagtgatctgcccacccaggcctctacagtgtttttgacataccctgaccatcacttttctgaaatggaaactctgggcattttttttttaaagcaatccttgcttttttgtgagtttgcagactttcagcatcttccaattgctgtatttacaattttggcctcaaaaagtattatttgggttttgagtcccaaaaataatagtagatactgctcaatgactggctatacatggtagcttctcctggagtgaggaaggcattcaaatggctgggcgcggtagctggcacctgtaatcccaacactttcggaggccaaggcaggcagatcatgaggtcaggagttcaagaccaggctgaccaacgtggtgaaacctcctcttttctaaaaatacaaaaattagctgggcgtggtggcgcaccgcctgtaatctcagctactgagagaatcgcttgaacctgggaggtggagtttgcagcgagtcgagattgcaccattgtactccatccagcctgggcaacggagcaagattccgttcccgcccccgccccaaaaaaaggcattgaagttaagatagactatatagttagatcctggacacacacatggattttgaggaatgatgtgagtttgtttatgtatgtacattttagcagttaacagatttggagtaaattgaatatttataaaacaacagtaattgccatgtaggtttactgtcatagtggaagatgattaagttgatggtacctacaggtattttgctatgaaaatgttttgacaaacaggatgatcgtgtaatttatgttccaagctctagtttgagaatggaagaatgtggtaaatttttgccaactgaacaggcataaagcgctgataataagggacttggccttaaggtaggaggttgttagcatttctttctaaacgtgtaagagtttatttaggtgacacccagcgttttggaaaaatgggtgtttgtttagaacaataatttggagggaagtggactaaacagggtttttagattaaggtttgtgtttatgtatctgtatctgcaaatactcagccataaatgtttctacctgtaagttgggtataatacaagctccacttggtatcaaaaaggactacccttagtgtcttccatgactagttatggaatgtaccagacctagagaggagttgttctaacctggagcttttgaaaatgtttccggtccataccctagaccaattaagtcagactgcagaataggactcagacatcagaattgtgaagctcccaggagatgtcaaggtatacccaagactgagaaccagtaccctgtctagactgaaccaggcttggttttagaagtattaaatctcgcctgggtacggtggctcacacctataatcccagcactttgggaggctgaggtgggtggatcatctgaggtcaggagagtgagaccagcctgtccaacatgttgaaaccccatctctactatacaaaattagccaggcatggtggtgcatgctgtaatcccagctacttgggaggctgaggcaggagaattgcttgaaccccggagatggaggttgcagtgagctgaggtcgcacgattgtgctccagcctgggcaaccagtgaaactctggggggaaagaaaaaaaatgtattaaatctctagttttagagagttacaccataacatccctgagtatggtcaatttcaagtcagctttagtgtgacaagcccctaggcccaccatttatgtctttatatattatggcaatatatgatccttatatattaaccactaatcgctgctgtttttgaatgttatcttttctgaggcagtcttgctctgtcacacaggctggagtgcagtaacaccactgaagctcactgtaactttgaactgctggacttggaatcctcctgctgtggccttcaaagggctgcaattacaagtgtgagccactgcatcccacctcacattttattctttggagattttttttgacttggattaaaaaactttatatttacacttca

SEQ ID NO: 60 - protein, 140 aa, Homo sapiens [sequence omitted]

SEQ ID NO: 61 - DNA, 5490 bp, Homo sapiens
  misc_feature: collagen type III alpha 1 chain (COL3A1)
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 62 - protein, 1466 aa, Homo sapiens
  misc_feature: collagen type III alpha 1 chain (COL3A1)
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQRMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 63 - DNA, 3610 bp, Homo sapiens
  misc_feature: ATP synthase F1 subunit epsilon (ATP5F1E)
cttcctgcggctgaaccgcccggctgagccgacattgccggcgtcttggcgattcggcccgacgagctccgctttcgctacagcatggtggcctactggagacaggctggactcagctacatccgatactcccagatctgtgcaaaagcagtgagagatgcactgaagacagaattcaaagcaaatgctgagaagacttctggcagcaacgtaaaaattgtgaaagtaaagaaggaataatctaccctgactaaagcttgaaatgctacatttccaaggtgaagatgtgtgggcacatgttatggcagattgaaaaggatctcattccatgggaaaaaaaaaaatcctgtcttgttcataaattgacaatgtcaataaattgaaatatggttcactgttactcttgattttagtttgtgagactttttcattttctttaaaggaactgtctgcacagctgcatgattatggccatgtgaaccagggtttcttaaaagcaggggaagccagatttaatatttagtgaccaaagggatacagaaagcattttgaaaatgtctcaaaatagttgtttcatgacattaattggtgatcaaaattatctgctgtcattgaggcttcgtagtcccttatttttgaattgttgtcatgtcagaataatgacatggttatcagtaacaaatggaaataattattaaatactaaaagtgtagattagaagaagaaatactaaattgtacttgttttgtattgtgcacttaaagcctatagataagaacggttacagaatttaagcacaaagtgtttttcacattgttttgtaaatgaatctttatactctggtagcatttctcataggaaatgaggcaaagctattgaataacttgctcattgagttgatggccaaactgaaattaaaacctaggagttttgactgcaagtctgggacacggtgaccacaacagcagtttcgtggtatttccatagtgattcattcattcattatttagggatgaaaaattgttgcaagttatgtttttatctttctttgaaacttgaaagggcttgggcaagtaaaacaccattgcaaagttaactgcctgattttaactgtttttggagactggagaaataaatttgaactggaaattttgaaacaccattctaggatcagttctcacatggtacttaagctcacaatgatttatcagggccactgtcagcaatgccgaggataagccgaaaagaacatctttgttctcggcttctgggaatgacttattgtcccgacacttgacctgtaggttttactgaattctcagcacagagcgagcagggaggcagagccaattgagtggaagagggtgcctctggaaaacagcaggttaggagcccttggagtcctccgcctcggagctgtcatcctgctgttggcaagttagggcacctcagttcagtgctgtggcccccaagattaaggtactaggaagtatcctccattacagggaacaggaataaatgaatcaaaaagctatcgggcctttttgacatgggacgaataaagtaactaataggcaaaggggctaaatgatttccagtcaagtaattagagataagtcatttattaaggaggcagatggggtattattggtatatagatggcatagtagattgctagtatattggtagattattggtatatagatggtatgttaaggaggcagatggggtattactggtatatagatggtatggtagattggtagtatattggcagattattggtatatggtatgttgaggcagatggggtattactggtatatagatggcatagtagattggtagtatattggtagattattggtatagagatggtatattattaaggaggcagatggggtaattagaggtgagacttagggacagggcttgtgatatggaaaggagtgaagtcactgagtaaaagacaagtttactgtcagcagagattcttagaggctcttttctccgtgattaaaaaaaaatcctaaaccagtagtataataaacttcctgcctccagcagtgctgggtagctggcaggatgactgtgcccagtgatgaggtgaccccagtcagaccaacttaatgagtatcccactaattagttggatgatgtttgaagctgtcagttaatctcttagttcacatcgttcttgtgagcaaagtgaaggaatcggataagctctaaatttcggtgatgctgctgctctgagagaccaaggtcgctggagcaaccgtactgctaaaggataagctcttcaaaatcaaaagggacagatttcttaagtttctagtttgttttgtgtttttacatctatttctaccatacgaaagagtaggatggtgagatgcatttctaaatatttttaactactcagtctggatggttaggcttcaagaaaaggaatttatttagtctgaaataaagcaattttgccatttaaaattgaattactttgtagtttaatgattactattgagcaaacaaatctttacagtaatataagtcagcaaaagttttttaaaaggtttaaatccctccttccttacatctatttcattgcttttttaggtttgggaagtaaagcaccagaggaagcatgtgtaaatccttgtataatcgcaggctgtaagtctttagcatctcaggaagttactaacttcaaactaagtatataggtagagtttcttactaaatctagtgcttcttgaaccacaagtagaaagcatttaaaacatgaatgttgttttgtgttttttgaagtttgtaaatagaagacttgttgatgatccgatggcaaggtatttttctcttggtatgtattttagttatttcctcgtgatgcataagtgaaaagagtgaagtttctcagaatgagcaactgtcatccatctacctgctattttattattgctgattacaaaagcaaatcaagagatgagaacccagttgcctgcaagtaaatatttactgcattgagggtcggagcattttcccattaccggttatccatggatcaaatagtgtatctcagtggtaattctagagggccattaaaaccctgatggtgctggaagagatggcagtgctgcatgtcagaaataggtaaactgtaattaagaagttacagatgatttgattacgctcttgtgtatttggtcctgttataatgtgagcagattaaaatcatgtagtgcttaaagctatgtcattatgcaaacatttatggcaacttctgcaaattaatttgaactgtaaaagttccctaatgagacgatgtcctccataactgagaaggacactgcagccattgctgcttaattcctgcatttgaatggttgccgagcgcatggtgtgccctttctcaaagagcagagaggctgcagagcattttggggggtgatgactgaatgaaagtgaaaagaccatttcaaaatagcctttaaaaggaaagccaaggatgattattaatgaattcctgggatctaacagtagtaagaaatttaaactatataaacatatatgttaaaggctagctcttcagaaataaagatcagaagactgca

SEQ ID NO: 64 - protein, 51 aa, Homo sapiens
  misc_feature: ATP synthase F1 subunit epsilon (ATP5F1E)
MVAYWRQAGLSYIRYSQICAKAVRDALKTEFKANAEKTSGSNVKIVKVKKE

SEQ ID NO: 65 - DNA, 778 bp, Homo sapiens
  misc_feature: ribosomal protein s8 (rps8)
gttttacaaaccgaaccgtgaatctttgcggtttctctttccagccagcgccgagcgatgggcatctctcgggacaactggcacaagcgccgcaaaaccgggggcaagagaaagccctaccacaagaagcggaagtatgagttggggcgcccagctgccaacaccaagattggcccccgccgcatccacacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgactcctgaggaagaagagattttaaacaaaaaacgatctaaaaaaattcagaagaaatatgatgaaaggaaaaagaatgccaaaatcagcagtctcctggaggagcagttccagcagggcaagcttcttgcgtgcatcgcttcaaggccgggacagtgtggccgagcagatggctatgtgctagagggcaaagagttggagttctatcttaggaaaatcaaggcccgcaaaggcaaataaatccttgttttgtcttcacccatgtaataaaggtgtttattgttttgttcccacatttatgttgcctgaatatatgactgttttctctgcttta

SEQ ID NO: 66 - protein, 208 aa, Homo sapiens [sequence omitted]

SEQ ID NO: 67 - DNA, 1291 bp, Homo sapiens
  misc_feature: ribosomal protein L31 (RPL31)
cttcctttccaacttggacgctgcagaatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaaatctacagacagtcaatgtggatgagaactaatcgctgatcgtcagatcaaataaagttataaaattgccttcatgtttttgttctttttagttgcaacataatgtacttgtataccctatcctaattatgggatcatttgaagagcttttcccaaattgatggcctgtgctgccttctccccatcccctggggttttaaagtgatttcaaactgcaacctagttttagaaccactgttctgggtagttgggatactgaaggcatattgttaattattctacttgtatgttttgctaattctaagataagcatttttccagaaaccaggatgtagaatccagttgccattgacatcttaacattttaggaaacaactttaaaatgatatactatctatctatctatctgtagcatcttaaaggtaatgaaattaatgtggcagtaggtcttttaagcttctgcctacatccatattgagtatagttgttgtcttctaaaataattaattgatttttggtgagataaccagattcatattttaagccttttgtaatggccccgtggtacctggagtcaaggttcagaagtaaaaagttccttaaggtatcaataacaaaaatttgtattaatagttcagtcctaaagcagtgttgctgagattatgtttcaccagcatttacaagctgtatgttaaatgctgccataaagaggtctctgaagccgtagggcacacccaaggcagggctgagaagtacctagtagtgttgcaccacccaaaaaccatggatgggcagcagcaacatctccagcttacccattcactgccacagtctacagcttaagacacttaactacaaggtaaaagaaaaggaccaagtaaatacaaaaagtttcttattaaaaaacttggaagccaa

SEQ ID NO: 68 - protein, 125 aa, Homo sapiens [sequence omitted]

SEQ ID NO: 69 - DNA, 2861 bp, Homo sapiens
  misc_feature: poly(A) binding protein cytoplasmic 1 (PABPC1)
ccccttctccccggcggttagtgctgagagtgcggagtgtgtgctccgggctcggaacacacatttattattaaaaaatccaaaaaaaatctaaaaaaatcttttaaaaaaccccaaaaaaatttacaaaaaatccgcgtctcccccgccggagacttttattttttttcttcctcttttataaaataacccggtgaagcagccgagaccgacccgcccgcccgcggccccgcagcagctccaagaaggaaccaagagaccgaggccttcccgctgcccggacccgacaccgccaccctcgctccccgccggcagccggcagccagcggcagtggatcgaccccgttctgcggccgttgagtagttttcaattccggttgatttttgtccctctgcgcttgctccccgctcccctccccccggctccggcccccagccccggcactcgctctcctcctctcacggaaaggtcgcggcctgtggccctgcgggcagccgtgccgagatgaaccccagtgcccccagctaccccatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaaattgatcagggaccatgaaaagaaacttgtgcttcaccgaagaaaaatatctaaacatcgaaaaacttaaatattatggaaaaaaaacattgcaaaatataaaataaataaaaaaaggaaaggaaactttgaaccttatgtaccgagcaaatgccaggtctagcaaacataatgctagtcctagattacttattgatttaaaaacaaaaaaacacaaaaaaatagtaaaatataaaaacaaattaatgttttatagaccctgggaaaaagaattttcagcaaagtacaaaaatttaaagcattcctttctttaattttgtaattctttactgtggaatagctcagaatgtcagttctgttttaagtaacagaattgataactgagcaaggaaacgtaatttggattataaaattcttgctttaataaaaattccttaaacagtg

SEQ ID NO: 70 - protein, 636 aa, Homo sapiens
  misc_feature: poly(A) binding protein cytoplasmic 1 (PABPC1)
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 71 - DNA, 1330 bp, Homo sapiens
  misc_feature: ribosomal protein S28 (RPS28)
actcctctccgccagaccgccgccgcgccgccatcatggacaccagccgtgtgcagcctatcaagctggccagggtcaccaaggtcctgggcaggaccggttctcagggacagtgcacgcaggtgcgcgtggaattcatggacgacacgagccgatccatcatccgcaatgtaaaaggccccgtgcgcgagggcgacgtgctcacccttttggagtcagagcgagaagcccggaggttgcgctgagcttggctgctcgctgggtcttggatgtcgggttcgaccacttggccgatgggaatggtctgtcacagtctgctccttttttttgtccgccacacgtaactgagatgctcctttaaataaagcgtttgtgtttcaagttaactcaggttcttgtctgggttatacgactagggtttctccaggtttcttgagtggctcccaggcggtcaccgatcctccgcactctggaaatcctggccgtgcggtcttgccaaacgaagcttttcctttttgaggcggggggtcgtgtttgtcgattgcaccctctaccccaaacaaaacacaagcgtagtaggaatgttttattagcaaagaagtttcagagagtgggtggatcagggctctatcacttggtccccacctcaccttggtggggccagagtgagccccttcctgccacagtcaccccaactgaaattgcctttctcttcggccagtgttagcctctgagcaggggaccctggacccttctgtgcgccaaaggctgaggtgactgacgaggagatctccccacagctaggtgtagtgagccagacgaggcagcttactgaacctgggggttctctccattgtcaccgcattctccttcaccaggtgtggctgtctgggagccagggggtgactcgctctggagagaggggaaaagaggggggcctgctgcaatctccttgaggcaggaaacgtgggattcagccccagcctcacttagtggaggttcttttaccatggacccaggctgcctggtttgtatccaacctctgccccttctgacctggaagaggcgcttgaccttcctcccacatcccttccagtggggtgagtacaggtgttcctcagtttacaatgggttacattccggtgagtacatcataggttgaaagtattgcaagttgaaatgtgtttaatacacctaatctcccaaacatcacagcttagcatggtccatcttaagcttgttcagaacgccttagcctgtagttggggaaactcgtctaacacgaagcctgttttaataaagtattgaatgtcttatgtaatttattgaa

SEQ ID NO: 72 - protein, 69 aa, Homo sapiens [sequence omitted]

SEQ ID NO: 73 - DNA, 911 bp, Homo sapiens
  misc_feature: fatty acid binding protein 4 (FABP4)
acagcaccctcctgaaaactgcagcttccttctcaccttgaagaataatcctagaaaactcacaaaatgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccatgatttagcaagcaactaattttctcccaagctgattttattcaatatggttacgttggttaaataaactttttttagatttagaaggtgatgtaatgatgtattcattgtgcttatgatgtattcttagtcataactgagtgaaggaaatgggaaatttgcattatttctttgttctgatatgaataataacatatttcataataattcaaggtaaaaagggatatctatggatttccctaggtaggagataacaagtatgtaccattactgaatattaaatccttctttaccatagctacagttaagtaggtgtatctcagaaacctaaggtagttttaaatgtagtgaaattg

SEQ ID NO: 74 - protein, 132 aa, Homo sapiens
  misc_feature: fatty acid binding protein 4 (FABP4)
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 75 - DNA, 6850 bp, Homo sapiens
  misc_feature: decorin (DCN)
aactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgacttcatgtaatcaggctgaacatttctacaattactagatgtattagacgtaagtattttctttagttaaaccacccatgttagaaatgttttctgtagaatttataaacaactatcaatgcagacaatttaataagcctggggatgatttacttacagtaaacatttatcaaattgtacatttgtgctatcaacaattaataagcaaatatgtgaaaatagtttctgtcttctatgaagttagatatttgatggttaaaacccctataaatcatagtttcatatgggaaaaaataattgaaatacagtgtaaatttaaataatttattaagtatagcaaataattgaaatatggtggactaaattttgtcatagaaatatgtgcaagttatagtagtggctcacatgagaggtaatcaattctgctaatagtagcagaatgagtgcagtggaacatgaaaaacttgaggagataacagttgaggtgggtttccatagatgcataatagttcaagagcaagatttggtggggaggcactattcaagacagggactaagttcaaaatccaagacgtatgctgggacacacctctgacaggttggcataaaggaggcttaatcaaactatttttcttcttctgaaacagaagcaataattttcatttacatttgacatatcccgaggtaatattaacattagggaaagttactcttttccatctttccacattcttgcaggaccataaaatctgaattttccagtatttttaataagagggaagaaacctctctttttcttctctttttcatctcccaagagatcctcctctcatgactacagttgaataggtggtttctattggaagacattcaggaattcaaggtgcatgtccataaatggactttttttgttgttgttcagagctggaccttgaatgatgcatccttctctctgttgtaaccatgaataatgcacccttcatgctatagcctttaacgattcacccttcttattgtaaccttgaatgattcaccctttatggtgtagccttgagtgacgcacccttcatgttgtagccttcaatgatgcacactccatgttatagccttgaatgatataccctttatgctgcagcctttctcttatggggaaaagcctgcagatatcctgctgcttaactgacaagtgtggtgagaaataagtagaaatctaaagaggggaagaccattttggacacttatctgcaaggcagatccaacacacttttccagtagtcaagctacttctaattttgttcagtatcaaaatgagaaacaggcctgattctccagcactcttgtcaacacaacttccccccatatttatatatatacacacacatatatatctttatatatatacacacatatatatctttatatatatatatttatatatatatctttttgcatatatacatatatatgtatctttatttcctttgaaataaagataaatatagctgatttctttggcttcgacacttactatttgcatgactaagggaagctagttaacctttctgtgactcatttccttgtccataaaatgggaatattaattgtacatgtcttatggattggtgtgtgaattcagttagcgagtgtagaatataacttatagatcaaagtagagtaaatggaaagggctcaactatggtgttgctactgccattgttattacaggcacacagttcgagctataatcatttcaagggaaattcttatgtgtcagttctggatcgaggtctgagattctgcatttcaaacaaacttccaggaatgctgctgcttcttggtccacacttggagaaataagtcagcagagagtcctctcgtttcctattgtaccatgtctgtcttttgtctcctgcttattggcctctgtaaggaactcacagctgctataataaagtaccaaaaactgggtggcttaaaacaacagaaacttactttctcacaattctggaggctaaaaattcaaaatcaaggtgtcagcagggtcagattccttccaaagcctttaggagaggacctttccttgctcctcctaggtttctggttaaatctaggtgttctttgccttatggcagcgtgactctaatttatgcctccatcttcaccctcacatggccttctctctcatgtgtttgtgtcttttctcttcttcttctatgaacatgagttatattggattaaggctcaccctaatttagtatgaacccatcttaacttgattatatctgcaaagactctatttccaaatgaagtcagactcacaggtattgggggtttgatattgaacatatcttctgggaggagacacaacttaatcattaatatccactttcttttttccttattaaatgtttaatttttttgttttcattaaaactgttgttcgattatgggtgcttcacataaaaggttggaaacttaaaaatttgtctctgacccctcctggttggaaaggcctctgttgtacatttatgctagcctaggccataccactttctgtcttcagtacagccatcttagtttattcaagtgacacagattttccagaacacagtattcatgatcttttaaagcatttttcttcaaagactttgatctggcaataaatgttactatgtaattctcatgacataaattaggcataacttggatctcctcttcttctgctcattcatttgtctgaatcatcactattatcttttttatattcctttaccgttctccataatgcttttccaagaaactggtttattccacaattttattgcagaggcagctgcaggatatcataatcttatctttatacgaaggaagaattgcctaatccctcaagtaaactaaaaatgttttatacagtcatttctcattcatccaatgttccaggcagtcccagtccaagactgccccttcacacacacaacaactcttcacaagacttcactgtccttcagactctcctgcagcacagacattcgaggttgctgagtcgacattgcagttatttcactcttgattttgctgctcaattttaagtttccacacttattgctaaaacattccttctagtggatctgtttatgtattcaagcacaccatctgtcacaattatttcaaatcacaggtctgtgacagactgtcaatttcacaccacaaagtataatagtggaaaacaaagtaaaatcaagaaagggagaacactaaacttattaaaaccatatacaagcatacgcattcaggaaactaaaacaaccttcaaagaagttgaacaattttgagccaaaagaaagggcatatgcacaccctaccccagtataagataaccagaaaaggactgggtgagttgcctcctgagtattatgactgccatcaatcacttaggcattggagatcaggagactcttcccaggccacacattctgcagtgactgtgagggctgaatgaatgcttccaggccatcaggaacaagcatagaaacacaaatcaattttaaaatacactcaaaaatgtcaaaataatccaacttttttgtcttgatcagatatatctactagcacatattacttcattttttcagccaatatttattaataacctactgtgtgcttgacagtgttctagtggcagagacacagcagtgcataatgccttgccctctgaagcagatgttgcagcaggaggagacattccaggacatgacaattaaagtggaggaatgtcaagcagaactggggaataagggatgtgaagggggtgagacttgctattttatataggatggtcggaaaaaggacttatcaggagtgacatttgagcagggagatgaagaactgaagggagcaagccaagaaaagaggccctgaatttggaaaatacatggcacgttccaacaagagcagagaggacaaacgtgagcagagtaaagtgagcaagaagagataatatatgatacaaggtcagaaaggtaatgacaatcgcatcctgtaggactttttaggccattgtaaagatgagcttttactctgagtaagacagagagctattagaggggtctcggtagaggagtgacttgattcaactgtctttgagaggatccctttgcgtgtatagactgtaaggtaacaggaataggccaagagagaatatttcggatgctagtgcaataatcaggtgagagatgatgaagactttgacctggataatagtagaaaaattgttgagaagggatcaaaatatggggtttgttttgatggtagagaggccagggatggctgaatgatcagatggggcatgagagagaaagaaaagaaacagagatgacttcaggaattttggcctgggccactggaaggatgaagtcaccatttactgagatggtaatgactgggaggttgagctggaagaactgagaatcaaatatctggttttgaatctgttctttttgagatgcatattcaacttccatatggaggtgtcaaggaggatttagatctagaattttggagctcaagggaaagggttgagctgtagacataaattctagagatgccggaatatagattgtgatccttctttatcagcacagaaatgacttgactttgtccaaactaagcaatcatactgtacatgttagcaacacattttacagggccaatttggccttttgcaatgttctgtggtttctaagataaataaacatattatatgtttccctctgga

SEQ ID NO: 76 - protein, 359 aa, Homo sapiens
  misc_feature: decorin (DCN)
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 77 - DNA, 1650 bp, Homo sapiens
  misc_feature: matrix Gla protein (MGP)
gtaggagcctctctccctactgctgctacacaagaccctgagactgacctgcaggacgaaaccatgaagagcctgatccttcttgccatcctggccgccttagcggtagtaactttgtgttatgaatcacatgaaagcatggaatcttatgaacttaatcccttcattaacaggagaaatgcaaataccttcatatcccctcagcagagatggagagctaaagtccaagagaggatccgagaacgctctaagcctgtccacgagctcaatagggaagcctgtgatgactacagactttgcgaacgctacgccatggtttatggatacaatgctgcctataatcgctacttcaggaagcgccgagggaccaaatgagactgagggaagaaaaaaaatctctttttttctggaggctggcacctgattttgtatccccctgtagcagcattacagaaatacataggcttatatacaatgcttctttcctgtatattctcttgtctggctgcacccctttttcccgcccccagattgataagtaatgaaagtgcactgcagtgagggtcaaaggagagtcaacatatgtgattgttccataataaacttctggtgtgatactttcatcttgtaaatctgctttcttttgggaagatattgagatatttaaatcatggcccaccttacccaaaataggagattctgttcatctcatatctagtattaattagaaaaataactacataaaaagaaggaagctaagaaggcactcactcagccataaattctctaaaccctctctaccttggaatccgtgaatggaatctggtatgttttttgcaggattttcctattgtaaattgtggcaaatacagggctcccttcatttgcttttcatctcttatgcatcaaagtcaaaaacatttctgaatcaagataatctagaagagaaaaaaggaggaaaaggaaaagagaaagcagaagggacaaataaaagcaattggcaaaaactgtcaataatagtttatacacttaactatatcaataatcacattatatgtaaatagtctaaacaatccaattattttttacttctacttatgttatatttttacttctacatttgttaaaggttccacgctacatttttactattcttgctttaaataattttaataatttcttttaaagtttagataataagaaaatatcccgggccaggcacagtggccacacctgtaatctcagtagccatgaccataccaatgcactccagcctgtgaaacagagtgagactctgtctctacagaaaaataaaaaagaaaagaaagaaaagatctcatatatttacccatgtaattttcatttcctgttttcttcattcttctttccatctggtgtcacttcctttctgcctgacgacttcctttaacgttttttatagttcaggtctgcaggattctttaagttttgtatgcttgtttttattcttgaaagatattttcactgtatattgaatcccaagttgccacgtttcttttaattattttaaggtattccactgaattctagcttgcacaatttttcgatataaaatttgttgtaattcttacctttgttccactgtgtatactgcatcttttatctctggct

SEQ ID NO: 78 - protein, 103 aa, Homo sapiens
  misc_feature: matrix Gla protein (MGP)
MKSLILLAILAALAVVTLCYESHESMESYELNPFINRRNANTFISPQQRWRAKVQERIRERSKPVHELNREACDDYRLCERYAMVYGYNAAYNRYFRKRRGTK

SEQ ID NO: 79 - DNA, 2061 bp, Homo sapiens
  misc_feature: ribosomal protein L22 (RPL22)
cctttctaactccgctgccgccatggctcctgtgaaaaagcttgtggtgaaggggggcaaaaaaaagaagcaagttctgaagttcactcttgattgcacccaccctgtagaagatggaatcatggatgctgccaattttgagcagtttttgcaagaaaggatcaaagtgaacggaaaagctgggaaccttggtggaggggtggtgaccatcgaaaggagcaagagcaagatcaccgtgacatccgaggtgcctttctccaaaaggtatttgaaatatctcaccaaaaaatatttgaagaagaataatctacgtgactggttgcgcgtagttgctaacagcaaagagagttacgaattacgttacttccagattaaccaggacgaagaagaggaggaagacgaggattaaatttcatttatctggaaaattttgtatgagttcttgaataaaacttgggaaccaaaatggtggtttatccttgtatctctgcagtgtggattgaacagaaaattggaaatcatagtcaaagggcttcccttggttcgccactcatttatttgtaacttgacttctttttttttctgcttaaaaatttcaattctcgtggtaataccagagtagaaggagagggtgactttaccgaactgacagccattggggaggcagatgcgggtgtggaggtgtgggctgaaggtagtgactgtttgattttaaaaagtgtgactgtcagttgtatctgttgcttttctcaatgattcagggatacaaatgggcttctctcattcattaaaagaaaacgcgacatctttctaagattctctgtgggaaaatgactgtcaataaaatgcgggtttctgggccattcgtcttactttcattttttgattacaaatttctcttgacgcacacaattatgtctgctaatcctcttcttcctagagagagaaactgtgctccttcagtgttgctgccataaaggggtttggggaatcgattgtaaaagtcccaggttctaaattaactaaatgtgtacagaaatgaacgtgtaagtaatgtttctacaggtctttgcaacaaactgtcactttcgtctccagcagagggagctgtaggaatagtgcttccagatgtggtctcccgtgtggggcccagcaatgggggcccctgatgccaagagctctggaggttcttgaaagaggggacacgaaggaggagtgactgggaagcctcccatgccaaggaggtgggaggtgccctggaaatagctgcctcatgccacttaggccatgactggatttaatgtcagtggtgtgccacagtgcagaggctagacaactgaaaggggctaccaaggctgggaaaaaaatgcaattgttgctgtgagtgactttgaaagactctggtgccttgtggtgcccttctgaaattcaaacagtaatgcaaaagtgtctgcattagaatttacggtgtctaaaattcatgtttttaaaagagcttgcctacagatggtttccacacttgaaattgtgccctgcgagttgcatagctggaagttcaatgctcagtcctaccttggctcccattaaacatttggtgctctgtggattgagttgaacgtgttgaggctttgcaatttcacttgtgttaaaggctctggcatttttccatttctatgcaaatttctttgaagcagaattgcttgcatatttcttctctgccgtcacagaaagcagagtttctttcaaacttcactgaggcatcagttgctctttggcaatgtcccttaaccatgattattaactaagtttgtggcttgagtttacaaattctacttgttgcattgatgttcccatgtagtaagtcatttttagtttggttgtgaaaaaaccctgggctgaagttggcatttcagttaaaagaaaaaaagaaactagtcccagatttgaaaacttgtaataaaattgaaactcactggttttctatgtctttttgaactcttgtaatcgagttttgatcatattttctattaaagtggctaacacctggctactcttactgta

SEQ ID NO: 80 - protein, 128 aa, Homo sapiens [sequence omitted]

SEQ ID NO: 81 - DNA, 390 bp, Homo sapiens
  misc_feature: ribosomal protein L39 (RPL39)
ccctcctcttcctttctccgccatcgtggtgtgttcttgactccgctgctcgccatgtcttctcacaagactttcaggattaagcgattcctggccaagaaacaaaagcaaaatcgtcccattccccagtggattcggatgaaaactggaaataaaatcaggtacaactccaaaaggagacattggagaagaaccaagctgggtctataaggaattgcacatgagatggcacacatatttatgctgtctgaaggtcacgatcatgttaccatatcaagctgaaaatgtcaccactatctggagatttcgacgtgttttcctctctgaatctgttatgaacacgttggttggctggattcagtaataaatatgtaaggcctttctttttaa

SEQ ID NO: 82 - protein, 51 aa, Homo sapiens [sequence omitted]

SEQ ID NO: 83 - DNA, 1091 bp, Homo sapiens
  misc_feature: complement C1q A chain (C1QA)
actcctgctgggcagcccacagggtccctgggcggagggcaggagcatccagttggagttgacaacaggaggcagaggcatcatggagggtccccggggatggctggtgctctgtgtgctggccatatcgctggcctctatggtgaccgaggacttgtgccgagcaccagacgggaagaaaggggaggcaggaagacctggcagacgggggcggccaggcctcaagggggagcaaggggagccgggggcccctggcatccggacaggcatccaaggccttaaaggagaccagggggaacctgggccctctggaaaccccggcaaggtgggctacccagggcccagcggccccctcggagcccgtggcatcccgggaattaaaggcaccaagggcagcccaggaaacatcaaggaccagccgaggccagccttctccgccattcggcggaaccccccaatggggggcaacgtggtcatcttcgacacggtcatcaccaaccaggaagaaccgtaccagaaccactccggccgattcgtctgcactgtacccggctactactacttcaccttccaggtgctgtcccagtgggaaatctgcctgtccatcgtctcctcctcaaggggccaggtccgacgctccctgggcttctgtgacaccaccaacaaggggctcttccaggtggtgtcagggggcatggtgcttcagctgcagcagggtgaccaggtctgggttgaaaaagaccccaaaaagggtcacatttaccagggctctgaggccgacagcgtcttcagcggcttcctcatcttcccatctgcctgagccagggaaggaccccctcccccacccacctctctggcttccatgctccgcctgtaaaatgggggcgctattgcttcagctgctgaagggagggggctggctctgagagccccaggactggctgccccgtgacacatgctctaagaagctcgtttcttagacctcttcctggaataaacatctgtgtctgtgtctgctgaacatgagcttcagttgctactcggagcattgagagggaggcctaagaataataacaatccagtgcttaaga

SEQ ID NO: 84 - protein, 245 aa, Homo sapiens
  misc_feature: complement C1q A chain (C1QA)
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 85 - DNA, 1098 bp, Homo sapiens
  misc_feature: complement C1q B chain (C1QB)
agtaggctctcggctcctggtcccactgctgctcagcccagtggcctcacaggacaccagcttcccaggaggcgtctgacacagtatgatgatgaagatcccatggggcagcatcccagtactgatgttgctcctgctcctgggcctaatcgatatctcccaggcccagctcagctgcaccgggcccccagccatccctggcatcccgggtatccctgggacacctggccccgatggccaacctgggaccccagggataaaaggagagaaagggcttccagggctggctggagaccatggtgagttcggagagaagggagacccagggattcctgggaatccaggaaaagtcggccccaagggccccatgggccctaaaggtggcccaggggcccctggagccccaggccccaaaggtgaatcgggagactacaaggccacccagaaaatcgccttctctgccacaagaaccatcaacgtccccctgcgccgggaccagaccatccgcttcgaccacgtgatcaccaacatgaacaacaattatgagccccgcagtggcaagttcacctgcaaggtgcccggtctctactacttcacctaccacgccagctctcgagggaacctgtgcgtgaacctcatgcgtggccgggagcgtgcacagaaggtggtcaccttctgtgactatgcctacaacaccttccaggtcaccaccggtggcatggtcctcaagctggagcagggggagaacgtcttcctgcaggccaccgacaagaactcactactgggcatggagggtgccaacagcatcttttccgggttcctgctctttccagatatggaggcctgacctgtgggctgcttcacatccaccccggctccccctgccagcaacgctcactctacccccaacaccaccccttgcccaaccaatgcacacagtagggcttggtgaatgctgctgagtgaatgagtaaataaactcttcaaggccaagggacagtggtctaattcaactctgtgtcccagcacctggcacaccagaagtgccatgctcagaaatgttggttacatgaatgaatgaaccatgaatgaatgaaa

SEQ ID NO: 86 - protein, 253 aa, Homo sapiens
  misc_feature: complement C1q B chain (C1QB)
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 87 - DNA, 5941 bp, Homo sapiens
  misc_feature: myosin heavy chain 6 (MYH6)
agatagagagactcctgcggcccagattcttcaggattctccgtgaagggataaccaggggaagcaccaagatgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtcggcctacctcatggggctgaactcagctgacctgctcaaggggctgtgccaccctcgggtgaaagtgggcaacgagtatgtcaccaaggggcagagcgtgcagcaggtgtactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcaggaagcccacttctccctgatccactacgccggcactgtggactacaacatcctgggctggctggaaaaaaacaaggatcctctcaacgagactgttgtggccctgtaccagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgcatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaaggggacagagaagctgctcagctctctggacattgatcacaaccagtacaagtttggccacaccaaggtgttcttcaaggcagggctgcttgggctgctggaggagatgcgggatgagaggctgagccgcatcatcacgcgcatgcaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagacgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggttgaggaagacaaggtcaacagcctgtccaagtctaaggtcaagctggagcagcaggtggatgatctggagggatccctagagcaagagaagaaggtgcgcatggacctggagcgagcaaagcggaaactggagggcgacctgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttggcccggcagctagaggaaaaggaggcgctaatctcgcagctgacccgggggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtggaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggagcaggagctgattgagaccagcgagcgggtgcagctgctgcattcccagaacaccagcctcatcaaccagaagaagaagatggagtcggatctgacccagctccagtcggaagtggaggaggcagtgcaggagtgcagaaacgccgaggagaaggccaagaaggccatcacggatgccgccatgatggcagaggagctgaagaaggagcaggacaccagcgcccacctggagcgcatgaagaagaacatggagcagaccattaaggacctgcagcaccggctggacgaggccgagcagatcgccctcaagggaggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtgacactgcctcgggaacctcactcttgccaacctgtaataaatatgagtgcc

SEQ ID NO: 88 - protein, 1939 aa, Homo sapiens
  misc_feature: myosin heavy chain 6 (MYH6)
MTDAQMADFGAAAQYLRKSEKERLEAQTRPFDIRTECFVPDDKEEFVKAKILSREGGKVIAETENGKTVTVKEDQVLQQNPPKFDKIEDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQVLALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMESDLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 89 - DNA, 3451 bp, Homo sapiens
  misc_feature: secreted protein acidic and cysteine rich (SPARC), transcript variant 1
agactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaactgtattctggtgttctcttctacacagtgggagggcgagcagtaggagagattggcccatttggtgctggccatttgaggaatgcaagcccagcactagtctcataatctctaggaatctgtagagagaggaattgaagtaaatttcagcattggctcattcagtcattcggcgacattcatcaggtacctgcaatgtgttaggggatcttatgagtaggcagcgtgcgtgatccttgctcccctggagctttctaacattctagcaggcagaccacacataaatttgcaatactgtttctgataaaaacgtgctgtaaaggaaataaagcagagaactatcatggaaaa

SEQ ID NO: 90 - protein, 303 aa, Homo sapiens
  misc_feature: secreted protein acidic and cysteine rich (SPARC), transcript variant 1
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 91 - DNA, 561 bp, Homo sapiens
  misc_feature: translation machinery associated 7 homolog (TMA7), transcript variant 1
gtttccggtggcagggtctggggaagcggcggcaggcgccatgtccggccgcgaaggtggcaagaagaagccactgaaacagcccaagaagcaggccaaggagatggacgaggaagataaggctttcaagcagaaacaaaaagaggagcagaagaaactcgaggagctaaaagcgaaggccgcggggaaggggcccttggccacaggtggaattaagaaatctggcaaaaagtaagctgttccttgtgcctgaggagatggtgaccctttatttcatctgtatttaaacctctctattccctgccataacatcttttgccacgtatagctggaattaagtgttgtcttggagctgttgtacatttaagaataaacttttgtaaaaaaagaaaaatcttacagtggctcatcatctctttagttgttttcactaagtcgttcctaccataactgtgaatttaaagtaaaaccagctcagaatcttgccagagtctgttctttggtccttgttctaccctaaactttgtatcacctgaaattaaaccaactcatttgaaagga

SEQ ID NO: 92 - protein, 64 aa, Homo sapiens
  misc_feature: translation machinery associated 7 homolog (TMA7), transcript variant 1
MSGREGGKKKPLKQPKKQAKEMDEEDKAFKQKQKEEQKKLEELKAKAAGKGPLATGGIKKSGKK

SEQ ID NO: 93 - DNA, 970 bp, Homo sapiens
  misc_feature: ribosomal protein L23a (RPL23A)
attggagacccttttcacaagatggcgccgaaagcgaagaaggaagctcctgcccctcctaaagctgaagccaaagcgaaggctttaaaggccaagaaggcagtgttgaaaggtgtccacagccacaaaaagaagaagatccgcacgtcacccaccttccggcggccgaagacactgcgactccggagacagcccaaatatcctcggaagagcgctcccaggagaaacaagcttgaccactatgctatcatcaagtttccgctgaccactgagtctgccatgaagaagatagaagacaacaacacacttgtgttcattgtggatgttaaagccaacaagcaccagattaaacaggctgtgaagaagctgtatgacattgatgtggccaaggtcaacaccctgattcggcctgatggagagaagaaggcatatgttcgactggctcctgattacgatgctttggatgttgccaacaaaattgggatcatctaaactgagtccagctgcctaattctgaatatatatatatatatatcttttcaccatatacatgcctgtctgtcaatttctggttgggctgggaggccacacacacacactgacatgacagggcttgggcaagactcctgttctacttatccttttgaaatacctcaccctgccactccaccatgtatgatcattccagagatctttgtgactagagttagtgtcctaggaaaaccagaactcagaacttgcctccatggttgagtaacaagctgtacaagaaccccttttatccctggaagaggctgtgtatgaaaccaatgcccagggtttgaagggtgttagcatccatttcaggggagtgtggattggctggctctctggtagcattttgtcctcacacacccatctactatgtccaaccggtctgtctgcttccctcaccccttgcccaataaaggacaaggacttcagaggagta

SEQ ID NO: 94 - protein, 156 aa, Homo sapiens [sequence omitted]

SEQ ID NO: 95 - DNA, 421 bp, Homo sapiens
  misc_feature: NADHubiquinone oxidoreductase subunit A1 (NDUFA1)
gcttgctgggaagagaggcgaagccaggtcacctttcaaggacccagaagtagggttttggcctaggtaacggggcagagatgtggttcgagattctccccggactctccgtcatgggcgtgtgcttgttgattccaggactggctactgcgtacatccacaggttcactaacgggggcaaggaaaaaagggttgctcattttgggtatcactggagtctgatggaaagagataggcgcatctctggagttgatcgttactatgtgtcaaagggtttggagaacattgattaaggaagcattttcctgattgatgaaaaaaataactcagttatggccatctacccctgctagaaggttacagtgtattatgtagcatgcaatgtgttatgtagtgcttaataaaaataaaatgaaaaaaa

SEQ ID NO: 96 - protein, 70 aa, Homo sapiens
  misc_feature: NADHubiquinone oxidoreductase subunit A1 (NDUFA1)
MWFEILPGLSVMGVCLLIPGLATAYIHRFTNGGKEKRVAHFGYHWSLMERDRRISGVDRYYVSKGLENID

SEQ ID NO: 97 - DNA, 629 bp, Homo sapiens
  misc_feature: cytochrome c oxidase subunit 7C (COX7C)
cttttcagtccttgcgcaccggggaacaaggtcgtgaaaaaaaaggtcttggtgaggtgccgccatttcatctgtcctcattctctgcgcctttcgcagagcttccagcagcggtatgttgggccagagcatccggaggttcacaacctctgtggtccgtaggagccactatgaggagggccctgggaagaatttgccattttcagtggaaaacaagtggtcgttactagctaagatgtgtttgtactttggatctgcatttgctacacccttccttgtagtaagacaccaactgcttaaaacataaggatgtttcagttcctccatttaacagatatgaagagcattttaagaggtgcagcctctggaagtggatcaaactagaactcatatgccatactagatatgtttgtcaataaacttatgacgtgaatgcttaatgcctcttttttgaaatagggaatgtaataattggccatttgcctactttattatttgggtaacattccagtattactctctgtgatttagcttatttaatggtgttaaactgaggttatattaaatttttgattcccaggtcaggattttgttggtaatttatataataaaagggaaatacaaatcga

SEQ ID NO: 98 - protein, 63 aa, Homo sapiens
  misc_feature: cytochrome c oxidase subunit 7C (COX7C)
MLGQSIRRFTTSVVRRSHYEEGPGKNLPFSVENKWSLLAKMCLYFGSAFATPFLVVRHQLLKT

SEQ ID NO: 99 - DNA, 1203 bp, Homo sapiens
  misc_feature: ferritin heavy chain 1 (FTH1)
gccagacgttcttcgccgagagtcgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtacccaggtgttgtctttgaggtcttgggatgaatcagaaatctatccaggctatcttccagattccttaagtgccgttgttcagttctaatcacactaatcaaaaagaaacgagtatttgtatttattaaactcattagtttgggcagtatactaaggtgtggctgtcttggattcagatagaactaagggttcccgactctgaatccagagtctgagttaaatgtttccaatggttcagtctagctttcacagtttttatgaataaaaggcattaaaggctgaa

SEQ ID NO: 100 - protein, 183 aa, Homo sapiens
  misc_feature: ferritin heavy chain 1 (FTH1)
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 101 - DNA, 2203 bp, Homo sapiens
  misc_feature: 40S ribosomal protein S15a (RPS15A) Variant 2
ctctttccgccatctttccgcgccggtgagtagcactctctgagagctccaatttcatccgtctgccatcggcgccatcctgcaatctaagccacaatggtgcgcatgaatgtcctggcagatgctctcaagagtatcaacaatgccgaaaagagaggcaaacgccaggtgcttattaggccgtgctccaaagtcatcgtccggtttctcactgtgatgatgaagcatggttacattggcgaatttgaaatcattgatgaccacagagctgggaaaattgttgtgaacctcacaggcaggctaaacaagtgtggggtgatcagccccagatttgacgtgcaactcaaagacctggaaaaatggcagaataatctgcttccatcccgccagtttggtttcattgtactgacaacctcagctggcatcatggaccatgaagaagcaagacgaaaacacacaggagggaaaatcctgggattctttttctagggatgtaatacatatatttacaaataaaatgcctcatggactctggtgcttccacttggtcgttttgagcctttacagcagtgtagccacagcttctgcggcagcatgcagttgcttcgtttatcggtgaatgcgattccctgaagtgactaatacagccaagggaaaaagttcttatgaaaccagtatgcctaagaaacagtcacccctgctgtctgccaaaaccaggtatttgacactaaatattttagttgtatttcagtttttttttttttttttcttttttggagacagagtctgactctattgctcaggctggagtgcagtggcgcgatcttggcccactgcaacctccacctcccgggttcaagtgattgtcctgtctcagccgcctgagtaactgggattacaagtgtgtgtcgccacatccggctaatttttatattttagtagagacagggtttcgccatgttgcccaggctggtcttgagttctgggcctcaagtgatcagcctacctcggcctcccgaagtgctgggattacagccacgagccattacacctggcctatatttcagtattttctattagtttttgatgaatttgttttgcctggctaggattattctgtagataggattttagatctggcttttgtcactgactgctgtaataaatacttgctaggaatttttttttttttttttttttttttaagacaaagtcgctctgtcacacaggctggagtgcagtggcatgatcttggctcactgcacctccgcttcccagattcaagtagttctcgtgcctcagcctcctgagtagctgggattacaggtgtgtgccaccatgtctggctgatttttgtatttttagtagaggtggggtttcaccatgttggccaggctggtctcgaactcctgatctcaagtgatctgcctgccttggtctcccaaagtgctgggattacaggtgtgaccaccacgccctgccttaagaattgttccaagagaatctggtgccacttgcaggtgcccattgaagtgcaatgggcactgttgatcactgaggaggtagtgggtgctgacccggtgctggggcctgtcccctagtctctgctttgcccttggctagctaggtggtgtgccaagtggggagagaagctaccttattaaggggcatggatcagcttcctgaaaggagggcctgcctctgtaagatatgggaagtcgctgagaatgttacagaacggccctagagatggggcagataacggcccccatttgtgagaagtgagttgggaggcatgtttggggcctctgatgtttgggaggctgtgggtaattaacatgagttttggggtccagcagcagaattcaggtttcctcttccactcagtaacctcagcatccgtatctgtaatgggaatgatacaaaacctatccccaagttgagggaaaaatgagattgtgtaaagcgcacttggcacatgacagtcacaagcatggggacagtgagtccagaaggattttcttatgccagcattgtaagccctaggatcacagggctctggcttgtttaaccatcgtgtctctggtccctagcctgcaaacctggtgtgtagggatgcctcagtcgcttacatgttgattgagtgaatcgtcggtttctttctggacactgacttcaaaaataaaataggatatgaaaatgg

SEQ ID NO: 102 - protein, 130 aa, Homo sapiens [sequence omitted]

(19) United States
(12) Patent Application Publication
(10) Pub. No.: US 2021/0198741 A1
(43) Pub. Date: Jul. 1, 2021

(54) IDENTIFICATION OF SPATIAL BIOMARKERS OF HEART DISORDERS AND METHODS OF USING THE SAME

(71) Applicant: 10x Genomics, Inc., Pleasanton, CA (US)

(72) Inventor: Stephen R. Williams, Pleasanton, CA (US)

(21) Appl. No.: 17/132,694

(22) Filed: Dec. 23, 2020

Related U.S. Application Data
(60) Provisional application No. 62/955,089, filed on Dec. 30, 2019.

(51) Int. Cl. C12Q 1/6883; G01N 33/68

(52) U.S. Cl. CPC C12Q 1/6883; G01N 2800/32; G01N 33/6893

(57) ABSTRACT

Provided herein are methods of detecting biomarkers and/or candidate biomarkers for heart disorders and uses of the same.

This application claims priority and benefit from U.S. Provisional Patent Application 62/955,089, filed Dec. 30, 2019, the contents and disclosures of which are incorporated herein by reference in its entirety.

### REFERENCE TO SEQUENCE LISTING

This instant application contains a Sequence Listing which has been submitted electronically in ASCII format and is hereby incorporated by reference in its entirety. Said ASCII copy, created on Dec. 15, 2020, is named Sequence_Listing_47706-0156001.txt and is 267,518 bytes in size.

### BACKGROUND

Heart disease is the leading cause of death in the United States. About 610,000 people die of heart disease in the United States every year, which corresponds to approximately 1 in every 4 deaths (CDC, NCHS. Underlying Cause of Death 1999-2013 on CDC WONDER Online Database, 2015). Diagnosis and treatment for heart disorders (such as heart disease) is challenging due to the complicated causes, symptoms, and types of heart disorders.

While biomarkers are useful tools in the diagnosis and prognosis of heart diseases, it has been difficult to identify biomarkers of heart diseases and exploit their use in clinical practice (Piek A e al., Critical Reviews in Clinical Laboratory Sciences, 55:4, 246-263, 2018). One of the reasons is that most studies ignore a spatial aspect of biomarker expression. Identifying biomarkers of heart diseases based on spatial expression would facilitate better diagnosis and treatment of such conditions.

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

### SUMMARY

This application is based on the discovery that a comparison of gene expression at a location in heart tissue can be used to identify diagnostic biomarkers, candidate drug targets, candidate prognostic biomarkers, and candidate biomarkers for determining efficacy of a treatment of a heart disorder. The methods of this disclosure solve the problem of identifying spatial biomarkers of heart diseases.

Featured herein is a method of assessing expression levels of one or more analytes in a subject having a heart disease or disorder, comprising obtaining a biological sample from the subject; and b) determining an expression level of an analyte. In some embodiments, the analyte is selected from one or more analytes selected from the group consisting of ribosome protein L17 (RPL17), ribosomal protein L36a (RPL36A), thymosin beta 4 X-linked (TMSB4X), coiled-coil domain containing 80 (CCDC80), ferritin light chain (FTL), ribosomal protein L37 (RPL37), heme oxygenase 1 (HMOX1), Y-box binding protein 1 (YBX1), ribosomal protein S17 (RPS17), ribosomal protein S29 (RPS29), pancreatic progenitor cell differentiation and proliferation factor (PPDPF), Y-box binding protein 3 (YBX3), vimentin (VIM), NADH:ubiquinone oxidoreductase subunit B1 (NDUFB1), ribosomal protein L34 (RPL34), macrophage migration inhibitory factor (MIF), phospholipid transfer protein (PLTP), ribosomal protein L38 (RPL38), ribosomal protein S21 (RPS21), ribosomal protein S10 (RPS10), ribonuclease A family member 1, pancreatic (RNASE1), ribosomal protein S25 (RPS25), ribosomal protein L26 (RPL26), ribosomal protein L37a (RPL37A), ribosomal protein 515a (RPS15A), eukaryotic translation elongation factor 1 alpha 2 (EEF1A2), ribosomal protein S27 (RPS27), nexilin F-actin binding protein (NEXN), collagen type I alpha 1 chain (COL1A1), ribosomal protein L23 (RPL23), collagen type III alpha 1 chain (COL3A1), ATP synthase F1 subunit epsilon (ATP5F1E), ribosomal protein S8 (RPS8), ribosomal protein L31 (RPL31), poly(A) binding protein cytoplasmic 1 (PABPC1), ribosomal protein S28 (RPS28), fatty acid binding protein 4 (FABP4), decorin (DCN), matrix Gla protein (MGP), ribosomal protein L22 (RPL22), ribosomal protein L39 (RPL39), complement C1q A chain (C1QA), complement C1q B chain (C1QB), myosin heavy chain 6 (MYH6), secreted protein acidic and cysteine rich (SPARC), translation machinery associated 7 homolog (TMA7), ribosomal protein L23a (RPL23A), NADH:ubiquinone oxidoreductase subunit A1 (NDUFA1), cytochrome c oxidase subunit 7C (COX7C), ferritin heavy chain 1 (FTH1), and byproducts, precursors, and degradation products thereof. In some embodiments, the analyte is selected from one or more analytes selected from the group consisting of ribosomal protein L36a (RPL36A), ferritin light chain (FTL), ribosomal protein L37 (RPL37), ribosomal protein S17 (RPS17), ribosomal protein S29 (RPS29), pancreatic progenitor cell differentiation and proliferation factor (PPDPF), Y-box binding protein 3 (YBX3), ribosomal protein L34 (RPL34), ribosomal protein L38 (RPL38), ribosomal protein S21 (RPS21), ribosomal protein S10 (RPS10), ribosomal protein S25 (RPS25), ribosomal protein L26 (RPL26), ribosomal protein L37a (RPL37A), ribosomal protein 515a (RPS15A), ribosomal protein S27 (RPS27), ribosomal protein L23 (RPL23), ATP synthase F1 subunit epsilon (ATP5F1E), ribosomal protein S8 (RPS8), ribosomal protein L31 (RPL31), ribosomal protein S28 (RPS28), fatty acid binding protein 4 (FABP4), ribosomal protein L22 (RPL22), ribosomal protein L39 (RPL39), complement C1q B chain (C1QB), translation machinery associated 7 homolog (TMA7), ribosomal protein L23a (RPL23A), NADH:ubiquinone oxidoreductase subunit A1 (NDUFA1), cytochrome c oxidase subunit 7C (COX7C), ferritin heavy chain 1 (FTH1), and byproducts, precursors, and degradation products thereof.

In some instances, the method further includes serially obtaining a biological sample from the subject at a plurality of time points. In some instances, the method also includes determining the expression levels of the one or more analytes in the serially obtained biological samples from the subject.

In some embodiments, disclosed herein is a method of diagnosing a subject as having a heart disease or disorder or having an increased likelihood of developing a heart disease or disorder, wherein the method comprises: (a) determining an expression level of one or more analytes selected from the group consisting of (1) RPL17, (2) RPL36A, (3) TMSB4X, (4) CCDC80, (5) FTL, (6) RPL37, (7) HMOX1, (8) YBX1, (9) RPS17, (10) RPS29, (11) PPDPF, (12) YBX3, (13) VIM, (14) NDUFB1, (15) RPL34, (16) MIF, (17) PLTP, (18) RPL38, (19) RPS21, (20) RPS10, (21) RNASE1, (22) RPS25, (23) RPL26, (24) RPL37A, (25) RPS15A, (26) EEF1A2, (27) RPS27, (28) NEXN, (29) COL1A1, (30) RPL23, (31) COL3A1, (32) ATP5F1E, (33) RPS8, (34) RPL31, (35) PABPC1, (36) RPS28, (37) FABP4, (38) DCN, (39) MGP, (40) RPL22, (41) RPL39, (42) C1QA, (43) C1QB, (44) MYH6, (45) SPARC, (46) TMA7, (47) RPL23A, (48) NDUFA1, (49) COX7C, (50) FTH1, and byproducts, precursors, and degradation products thereof, in a biological sample from the subject; and (b) identifying the subject having: (1) an elevated expression level of the one or more analytes (19)-(50), and byproducts, precursors, and degradation products thereof, of step (a), as compared to the reference expression level(s) of the one or more analytes (19)-(50) and byproducts, precursors, and degradation products thereof; or (2) about the same or a decreased expression level of the one or more analytes (1)-(18), and byproducts, precursors, and degradation products thereof, of step (a), as compared to the reference expression level(s) of the one or more analytes (1)-(18) and byproducts, precursors, and degradation products thereof; as having a heart disease or disorder or having an increased likelihood of developing a heart disease or disorder.

In some embodiments, disclosed herein is a method of diagnosing a subject as having a heart disease or disorder or having an increased likelihood of developing a heart disease or disorder, wherein the method comprises: (a) determining an expression level of one or more analytes selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, in a biological sample from the subject; and (b) identifying the subject having: (1) an elevated expression level of the one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, of step (a), as compared to the reference expression level(s) of the one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1 and byproducts, precursors, and degradation products thereof; or (2) about the same or a decreased expression level of the one or more analytes RPL36A, FTL, RPL37, RPS17, RPS29, PPDPF, YBX3, RPL34, RPL38, and byproducts, precursors, and degradation products thereof, of step (a), as compared to the reference expression level(s) of the one or more analytes RPL36A, FTL, RPL37, RPS17, RPS29, PPDPF, YBX3, RPL34, RPL38, and byproducts, precursors, and degradation products thereof; as having a heart disease or disorder or having an increased likelihood of developing a heart disease or disorder.

In some embodiments, the heart disease or disorder is myocardial infarction.

In some embodiments, the methods of diagnosing a heart disease or disorder further comprise obtaining the biological sample from the subject.

In some embodiments, the methods of diagnosing a heart disease or disorder further comprise confirming a diagnosis of myocardial infarction in the subject by obtaining an image of the subject's heart or performing cardiac testing on the subject.

In some embodiments, the methods further comprise monitoring risk of having a myocardial infarction in a subject over time, wherein the method comprises: (a) determining a first expression level of one or more analytes selected from the group consisting of (1) RPL17, (2) RPL36A, (3) TMSB4X, (4) CCDC80, (5) FTL, (6) RPL37, (7) HMOX1, (8) YBX1, (9) RPS17, (10) RPS29, (11) PPDPF, (12) YBX3, (13) VIM, (14) NDUFB1, (15) RPL34, (16) MIF, (17) PLTP, (18) RPL38, (19) RPS21, (20) RPS10, (21) RNASE1, (22) RPS25, (23) RPL26, (24) RPL37A, (25) RPS15A, (26) EEF1A2, (27) RPS27, (28) NEXN, (29) COL1A1, (30) RPL23, (31) COL3A1, (32) ATP5F1E, (33) RPS8, (34) RPL31, (35) PABPC1, (36) RPS28, (37) FABP4, (38) DCN, (39) MGP, (40) RPL22, (41) RPL39, (42) C1QA, (43) C1QB, (44) MYH6, (45) SPARC, (46) TMA7, (47) RPL23A, (48) NDUFA1, (49) COX7C, (50) FTH1, and byproducts, precursors, and degradation products thereof in a first biological sample obtained from a subject at a first time point; (b) determining a second expression level of the one or more analytes (1)-(50) and byproducts, precursors, and degradation products thereof of step (a), in a second biological sample obtained from the subject at a second time point; (c) identifying the subject as having: (1) an increased second expression level of the one or more analytes (19)-(50), byproducts, precursors, and degradation products thereof, of step (a), or (2) about the same or a decreased second expression level of one or more analytes (1)-(18) and byproducts, precursors, and degradation products thereof, of step (a), as compared to the first expression level of the one or more analytes (1)-(18) and byproducts, precursors, and degradation products thereof, as having an increasing risk of having a myocardial infarction, or (d) identifying the subject as having: (1) about the same or a decreased second expression level of one or more analytes (19)-(50) and byproducts, precursors, and degradation products thereof, of step (a), as compared to the first expression level of the one or more analytes (19)-(50), and byproducts, precursors, and degradation products thereof; or (2) an increased second expression level of one or more analytes (1)-(18) and byproducts, precursors, and degradation products thereof, of step (a), as compared to the first expression level of the one or more analytes (1)-(18) and byproducts, precursors, and degradation products thereof, of step (a), as having about the same or a decreasing risk of having a myocardial infarction.

In some embodiments, the methods further comprise monitoring risk of having a myocardial infarction in a subject over time, wherein the method comprises: (a) determining a first expression level of one or more analytes selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof in a first biological sample obtained from a subject at a first time point; (b) determining a second expression level of the one or more analytes RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1 and byproducts, precursors, and degradation products thereof of step (a), in a second biological sample obtained from the subject at a second time point; (c) identifying the subject as having: (1) an increased second expression level of the one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, byproducts, precursors, and degradation products thereof, of step (a), or (2) about the same or a decreased second expression level of one or more analytes RPL36A, FTL, RPL37, RPS17, RPS29, PPDPF, YBX3, RPL34, RPL38, and byproducts, precursors, and degradation products thereof, of step (a), as compared to the first expression level of the one or more analytes RPL36A, FTL, RPL37, RPS17, RPS29, PPDPF, YBX3, RPL34, RPL38, and byproducts, precursors, and degradation products thereof, as having an increasing risk of having a myocardial infarction, or (d) identifying the subject as having: (1) about the same or a decreased second expression level of one or more analytes (19)-(50) and byproducts, precursors, and degradation products thereof, of step (a), as compared to the first expression level of the one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof; or (2) an increased second expression level of one or more analytes RPL36A, FTL, RPL37, RPS17, RPS29, PPDPF, YBX3, RPL34, RPL38, and byproducts, precursors, and degradation products thereof, of step (a), as compared to the first expression level of the one or more analytes RPL36A, FTL, RPL37, RPS17, RPS29, PPDPF, YBX3, RPL34, RPL38, and byproducts, precursors, and degradation products thereof, of step (a), as having about the same or a decreasing risk of having a myocardial infarction.

In some embodiments, the methods disclosed herein further comprise administering a treatment of myocardial infarction to the subject.

In some embodiments, described herein are methods of determining efficacy of a treatment for reducing the risk of having a myocardial infarction in a subject, wherein the method comprises: (a) determining a first expression level of one or more analytes selected from the group consisting of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, in a first biological sample obtained from a subject at a first time point; (b) determining a second expression level of the one or more of the analytes of step (a) in a second biological sample obtained from the subject at a second time point, wherein the subject is administered one or more doses of a treatment for reducing the risk of having a myocardial infarction between the first and second time points; and (c) identifying: (la) the treatment as being effective in the subject having about the same or a decreased second expression level of one or more analytes RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, of step (a), or (1b) the treatment as being effective in the subject having an increased second expression level of one or more analytes RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, of step (a); or (2a) the treatment as not being effective in the subject having increased second expression level of one or more analytes RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, of step (a), or (2b) the treatment as not being effective in the subject having about the same or a decreased second expression level of one or more analytes RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof.

In some embodiments, described herein are methods of determining efficacy of a treatment for reducing the risk of having a myocardial infarction in a subject, wherein the method comprises: (a) determining a first expression level of one or more analytes selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, in a first biological sample obtained from a subject at a first time point; (b) determining a second expression level of the one or more of the analytes of step (a) in a second biological sample obtained from the subject at a second time point, wherein the subject is administered one or more doses of a treatment for reducing the risk of having a myocardial infarction between the first and second time points; and (c) identifying: (la) the treatment as being effective in the subject having about the same or a decreased second expression level of one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, of step (a), or (1b) the treatment as being effective in the subject having an increased second expression level of one or more analytes RPL36A, FTL, RPL37, RPS17, RPS29, PPDPF, YBX3, RPL34, RPL38, and byproducts, precursors, and degradation products thereof, of step (a); or (2a) the treatment as not being effective in the subject having increased second expression level of one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, of step (a), or (2b) the treatment as not being effective in the subject having about the same or a decreased second expression level of one or more analytes RPL36A, FTL, RPL37, RPS17, RPS29, PPDPF, YBX3, RPL34, RPL38, and byproducts, precursors, and degradation products thereof.

In some embodiments, described herein are methods of identifying a patient subpopulation for which a treatment for reducing the risk of having a myocardial infarction is effective, the method comprising: (a) determining (1) a first expression level of one or more analytes selected from the group consisting of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, in first biological samples obtained from a patient subpopulation at a first time point, and (2) a second expression level of the one or more of the analytes of step (a)(1), in second biological samples obtained from the patient population at a second time point, wherein the patient subpopulation is administered one or more doses of a treatment for reducing the risk of having a myocardial infarction between the first and second time points; and (b) determining a correlation between efficacy of the treatment for reducing the risk of having a myocardial infarction and the second expression level in the biological samples from the patient subpopulation as compared to biological samples obtained from an untreated patient, wherein (1) a lower second expression level of the one or more analytes RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, or (2) about the same or an elevated second expression level of the one or more analytes RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, in the biological samples from the patient subpopulation as compared to the biological samples from the untreated patient; is indicative that the treatment is effective for reducing risk of having a myocardial infarction in the patient subpopulation.

In some embodiments of the above methods, the treatment is (a) an antagonist of one or more analytes RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof; or (b) an agonist of one or more analytes RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof.

In some embodiments, described herein are methods of identifying a patient subpopulation for which a treatment for reducing the risk of having a myocardial infarction is effective, the method comprising: (a) determining (1) a first expression level of one or more analytes selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, in first biological samples obtained from a patient subpopulation at a first time point, and (2) a second expression level of the one or more of the analytes of step (a)(1), in second biological samples obtained from the patient population at a second time point, wherein the patient subpopulation is administered one or more doses of a treatment for reducing the risk of having a myocardial infarction between the first and second time points; and (b) determining a correlation between efficacy of the treatment for reducing the risk of having a myocardial infarction and the second expression level in the biological samples from the patient subpopulation as compared to biological samples obtained from an untreated patient, wherein (1) a lower second expression level of the one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, or (2) about the same or an elevated second expression level of the one or more analytes RPL36A, FTL, RPL37, RPS17, RPS29, PPDPF, YBX3, RPL34, RPL38, and byproducts, precursors, and degradation products thereof, in the biological samples from the patient subpopulation as compared to the biological samples from the untreated patient; is indicative that the treatment is effective for reducing risk of having a myocardial infarction in the patient subpopulation.

In some embodiments of the above methods, the treatment is (a) an antagonist of one or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof; or (b) an agonist of one or more analytes RPL36A, FTL, RPL37, RPS17, RPS29, PPDPF, YBX3, RPL34, RPL38, and byproducts, precursors, and degradation products thereof.

In some embodiments, any of the above methods further comprising administering a treatment for reducing the risk of having a myocardial infarction to the subject, adjusting a dosage of a treatment for reducing the risk of having a myocardial infarction for the subject, or adjusting a treatment for reducing the risk of having a myocardial infarction for the subject.

In some embodiments, the treatment further comprises administering at least one medication selected from a blood pressure lowering drug, and a cholesterol lowering drug, an anti-coagulant drug, an anti-platelet drug, a thrombolytic drug, nitroglycerin, and a pain-reliever.

In some embodiments, the (a) the blood pressure lowering drug is one or more drugs selected from the group consisting of a diuretic, a beta-blocker, an ACE inhibitor, an angiotensin II receptor blocker, a calcium channel blocker, an alpha blocker, an alpha-2 receptor agonist, a central agonist, a peripheral adrenergic inhibitor, and a vasodilator; (b) the cholesterol lowering drug is one or more drugs selected from the group consisting of a statin, a bile acid binding resin, a cholesterol absorption inhibitor, a fibrate, niacin, an omega-3 fatty acid, a combination cholesterol absorption inhibitor and statin, a combination stain and calcium channel blocker, and a monoclonal antibody; (c) the anti-coagulant drug is one or more drugs selected from the group consisting of apixaban (Eliquis), dabigatran (Pradaxa), edoxaban (Savaysa), fondaparinux (Arixtra), heparin (Fragmin, Innohep, and Lovenox), rivaroxaban (Xarelto), and warfarin (Coumadin, Jantoven); (d) the anti-platelet drug is one or more drugs selected from the group consisting of aspirin, clopidogrel (Plavix),dipyridamole (Persantine), ticlopidine (Ticlid), prasugrel (Effient), ticagrelor (Brilinta), vorapaxar (Zontivity); (e) the thrombolytic drug is one or more drugs selected from the group consisting of alteplase (Activase) and streptokinase (Streptase); and (f) the pain-reliever drug is one or more drugs selected from the group consisting of hydrocortisone (Cortef), methylprednisolone (Medrol), prednisolone (Prelone), prednisone (Deltasone), buprenorphine (Buprenex, Butrans), fentanyl (Duragesic), hydrocodone-acetaminophen (Vicodin), hydromorphone (Exalgo ER), meperidine (Demerol), oxycodone (OxyContin), oxymorphone (Opana), tramadol (Ultram), duloxetine (Cymbalta) and venlafaxine (Effexor XR), fluoxetine (Prozac) and paroxetine (Paxil), imipramine (Tofranil), nortriptyline (Pamelor), desipramine (Norpramin), carbamazepine (Tegretol), gabapentin (Neurontin), phenytoin (Dilantin), and pregabalin (Lyrica).

In some instances, (a) the vasodilator is one or more drugs selected from the group consisting of Bumetanide (Bumex), Chlorthalidone (Hygroton), Chlorothiazide (Diuril), Ethacrynate (Edecrin), Furosemide (Lasix), Hydrochlorothiazide HCTZ (Esidrix, Hydrodiuril, Microzide), Indapamide (Lozol), Methyclothiazide (Enduron), Metolazone (Mykroz, Zaroxolyn), Torsemide (Demadex), Minoxidil (Loniten), and Hydralazine (Apresoline); (b) the diuretic is one or more drugs selected from the group consisting of Chlorthalidone (Hygroton), Chlorothiazide (Diuril), Hydrochlorothiazide or HCTZ (Esidrix, Hydrodiuril, Microzide), Indapamide (Lozol), Metolazone (Mykrox, Zaroxolyn), Amiloride (Midamor), Bumetanide (Bumex), Furosemide (Lasix), Spironolactone (Aldactone), and Triamterene (Dyrenium); (c) the beta-blocker is one or more drugs selected from the group consisting of acebutolol (Sectral®), atenolol (Tenormin®), bisoprolol (Zebeta®), metoprolol (Lopressor®, Toprol XL®), nadolol (Corgard®), nebivolol (Bystolic®), propranolol (Inderal, InnoPran XL), carvedilol (Coreg), esmilol (Brevibloc), labetalol (Trandate, Normodyne), metoprolol tartrate (Lopressor) and metoprolol succinate (Toprol-XL), nadolol (Corgard), penbutolol sulfate (Levatol), sotalol (Betapace), and hydrochlorothiazide HCTZ and bisoprolol (Ziac); (d) the ACE inhibitor is one or more drugs selected from the group consisting of benazepril (Lotensin®), captopril (Capoten®), enalapril (Vasotec®), fosinopril (Monopril®), lisinopril (Prinivil®, Zestril®), moexioril (Univasc®), perinopril (Aceon®), quinapril (Accupril®), ramipril (Altace®), and trandolapril (Mavik®); (e) the angiotensin II receptor blocker is one or more drugs selected from the group consisting of azilsartan (Edarbi), candesartan (Atacand), eprosartan, mesylate (Teveten), irbesarten (Avapro), losartin potassium (Cozaar), olmesartan (Benicar), telmisartan (Micardis), and valsartan (Diovan); (f) the calcium channel blocker is one or more drugs selected from the group consisting of Amlodipine besylate (Norvasc, Lotrel), Clevidipine (Cleviprex), Diltiazem hydrochloride (Cardizem CD, Cardizem SR, Dilacor XR, Tiazac), Felodipine (Plendil), Isradipine (DynaCirc, DynaCirc CR), Nicardipine (Cardene SR), Nifedipine (Adalat CC, Procardia XL), Nimodipine (Nimotop, Nymalize), Nisoldipine (Sular), Verapamil hydrochloride (Calan SR, Isoptin SR, Verelan, and Covera HS); (g) the alpha-2 receptor agonist is one or more drugs selected from the group consisting of Methyldopa (Aldomet), Clonidine (Catapres®), Clonidine patch (Catapres-TTS®), Tizanidine (Zanaflex®), Clonidine (Kapvay®), Guanfacine (Intuniv®), and Lofexidine (Lucemyra™); (h) the central agonist is one or more drugs selected from the group consisting of clonidine hydrochloride (Catapres) and guanfacine hydrochloride (Tenex); (i) the peripheral adrenergic inhibitor is one or more drugs selected from the group consisting of guanadrel (Hylorel), guanethidine monosulfate (Ismelin), and reserpine (Serpasil); (j) the statin is one or more drugs selected from the group consisting of atorvastatin (Lipitor), fluvastatin (Lescol XL), lovastatin (Altoprev), pitavastatin (Livalo), pravastatin (Pravachol), and rosuvastatin (Crestor), and simvastatin (Zocor). (k) the bile acid binding resin is one or more drugs selected from the group consisting of cholestyramine (Prevalite), colesevelam (Welchol), and colestipol (Colestid); (l) the cholesterol absorption inhibitor is ezetimibe (Zetia); (m) the fibrates is one or more drugs selected from the group consisting of fenofibrate (Antara, Lipofen), and gemfibrozil (Lopid); (n) the omega-3 fatty acids is one or more drugs selected from the group consisting of lovaza, and icosapent ethyl (Vascepa); (o) the combination statin and calcium channel blocker is amlodipine-atorvastatin (Caduet); and (p) the monoclonal antibody is one or more antibodies selected from the group consisting of Alirocumab (Praluent) and Evolocumab (Repatha).

In some embodiments of any of the above methods, the treatment further comprises angioplasty, undergoing bypass surgery, and implanting a pacemaker or a defibrillator.

In some embodiments, the methods further comprise obtaining the first and second biological samples from the subject. In some embodiments, the biological sample(s) comprise blood, serum, plasma, or tissue. In some embodiments, the biological sample(s) comprise cardiac tissue.

In some embodiments, each of the first and second expression levels is an expression level of (a) protein or a byproduct or precursor or degradation product thereof or (b) an expression level of mRNA or a fragment thereof.

In some embodiments of any of the methods disclosed herein, the methods further comprise: (a) contacting the biological sample with an substrate comprising a plurality of attached capture probes, wherein a capture probe of the plurality comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

In some embodiments, the methods further comprise (a) contacting the biological sample with a plurality of analyte capture agents, wherein an analyte capture agent of the plurality of analyte capture agents comprises an analyte binding moiety and a capture agent barcode domain, wherein the analyte binding moiety specifically binds to the analyte, and wherein the capture agent barcode domain comprises an analyte binding moiety barcode and an analyte capture sequence; (b) hybridizing the capture agent barcode domain to a capture domain of a capture probe on an array, wherein the array comprises a plurality of capture probes, wherein the capture probe is part of the plurality of capture probes and comprises: (i) a spatial barcode and (ii) the capture domain; (c) extending a 3′ end of the capture probe using the capture agent barcode domain that is specifically bound to the capture domain as a template to generate an extended capture probe; and (d) amplifying the extended capture probe.

In some embodiments, the heart disease or disorder is selected from the group consisting of: congenital heart disease or disorder, arrhythmia, tachycardia, bradycardia, premature ventricular contraction, fibrillation, coronary artery disease (CAD), heart muscle disease, dilated cardiomyopathy, myocardial infarction, heart failure, hypertrophic cardiomyopathy, mitral regurgitation, heart valve disease, mitral valve prolapse, pulmonary stenosis, pericardial disease, heart infection, aneurysm, and sudden cardiac arrest.

In some embodiments of any of the above methods, the levels of at least two of the analytes are determined. In some embodiments, the levels of at least three of the analytes are determined. In some embodiments, the levels of at least four of the analytes are determined. In some embodiments, disclosed herein are methods for quantitatively profiling gene expression signatures correlating to a disease state of a subject, wherein the disease state is a heart disease, comprising: generating a profile of expression levels of a plurality of analytes, wherein an analyte in the plurality of analytes is correlated with the heart disease in a biological sample obtained from the subject, wherein the profile is generated from a library generated by: (a) contacting the biological sample with an substrate comprising a plurality of attached capture probes, wherein a capture probe of the plurality of attached capture probes comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

In some embodiments, the methods comprise administering an effective amount of a therapeutic agent to the subject, wherein the subject has been identified by profiling expression levels of a plurality of analytes, wherein an analyte in the plurality of analytes is correlated with the heart disease in a biological sample obtained from the subject, wherein the profile is generated from a library, wherein the library is generated by: (a) contacting the biological sample with an substrate comprising a plurality of attached capture probes, wherein a capture probe of the plurality comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

In some embodiments, the analyte of the plurality of analytes are selected from the group consisting of (1) RPL17, (2) RPL36A, (3) TMSB4X, (4) CCDC80, (5) FTL, (6) RPL37, (7) HMOX1, (8) YBX1, (9) RPS17, (10) RPS29, (11) PPDPF, (12) YBX3, (13) VIM, (14) NDUFB1, (15) RPL34, (16) MIF, (17) PLTP, (18) RPL38, (19) RPS21, (20) RPS10, (21) RNASE1, (22) RPS25, (23) RPL26, (24) RPL37A, (25) RPS15A, (26) EEF1A2, (27) RPS27, (28) NEXN, (29) COL1A1, (30) RPL23, (31) COL3A1, (32) ATP5F1E, (33) RPS8, (34) RPL31, (35) PABPC1, (36) RPS28, (37) FABP4, (38) DCN, (39) MGP, (40) RPL22, (41) RPL39, (42) C1QA, (43) C1QB, (44) MYH6, (45) SPARC, (46) TMA7, (47) RPL23A, (48) NDUFA1, (49) COX7C, (50) FTH1, and byproducts, precursors, and degradation products thereof.

In some embodiments, the analyte of the plurality of analytes are selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof.

In some embodiments, the heart disease is myocardial infarction.

In some embodiments, the methods further comprise determining (i) all or a portion of the sequence of the spatial barcode or the complement thereof, and (ii) all or a portion of the sequence of the analyte from the biological sample or the capture agent barcode domain.

In some embodiments, the methods further comprise using the determined sequences of (i) and (ii) to identify the location of the analyte in the biological sample.

In some embodiments, disclosed herein in a kit comprising (a) an antibody that binds specifically to one or more analytes selected from the group consisting of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof; and (b) instructions for performing the method of any one of the preceding claims.

Provided herein are methods for identifying a candidate drug target for treatment of a heart disease or disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as candidate drug target(s) for treatment of the heart disorder.

In some embodiments of any of the methods described herein, the method includes identifying one or more biomarker(s) showing elevated level(s) in the location in the sample as compared to reference level(s) of the one or more biomarker(s) in a corresponding location in a sample comprising heart tissue obtained from a control animal as candidate drug target(s) for treatment of the heart disorder. In some embodiments of any of the methods described herein, the method further includes testing the ability of an inhibitor of the expression and/or activity of the one or more identified candidate drug target(s) to treat the heart disorder in an animal.

In some embodiments of any of the methods described herein, the method includes identifying one or more biomarker(s) showing decreased level(s) in the location in the sample as compared to reference level(s) of the one or more biomarker(s) in a corresponding location in a sample comprising heart tissue obtained from a control animal as candidate drug target(s) for treatment of the heart disorder. In some embodiments of any of the methods described herein, the method further includes testing the ability of an agent that increases the expression and/or activity of the one or more identified candidate drug target(s) to treat the heart disorder in an animal.

Also provided herein are methods for identifying a diagnostic biomarker of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as diagnostic biomarker(s) of the heart disorder.

In some embodiments of any of the methods described herein, the method includes identifying one or more biomarker(s) showing elevated level(s) in the location in the sample as compared to reference level(s) of the one or more biomarker(s) in a corresponding location in a sample comprising heart tissue obtained from a control animal as diagnostic biomarker(s) of the heart disorder.

In some embodiments of any of the methods described herein, the method includes identifying one or more biomarker(s) showing decreased level(s) in the location in the sample as compared to reference level(s) of the one or more biomarker(s) in a corresponding location in a sample comprising heart tissue obtained from a control animal, as diagnostic biomarker(s) of the heart disorder.

Also provided herein are methods for identifying a candidate prognostic biomarker of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as candidate prognostic biomarker(s) of the heart disorder.

In some embodiments of any of the methods described herein, the method includes identifying one or more biomarker(s) showing elevated level(s) in the location in the sample as compared to reference level(s) of the one or more biomarker(s) in a corresponding location in a sample comprising heart tissue obtained from a control animal, as candidate prognostic biomarker(s) of the heart disorder. In some embodiments of any of the methods described herein, the method further includes performing an experiment to validate whether the one or more identified candidate prognostic biomarker(s) provides for an accurate assessment of the prognosis of the heart disorder in an animal.

In some embodiments of any of the methods described herein, the method includes identifying one or more biomarker(s) showing decreased level(s) in the location in the sample as compared to reference level(s) of the one or more biomarker(s) in a corresponding location in a sample comprising heart tissue obtained from a control animal, as candidate prognostic biomarker(s) of the heart disorder. In some embodiments of any of the methods described herein, the method further includes performing an experiment to validate whether the one or more identified candidate prognostic biomarker(s) provides for an accurate assessment of the prognosis of the heart disease disorder in an animal.

Also provided herein are methods for determining a candidate biomarker for determining efficacy of a treatment of a heart disorder that include: (a) determining level(s) of one or more biomarker(s) in a location in a sample comprising heart tissue obtained from an animal having a heart disorder; and (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as a candidate biomarker for determining efficacy of a treatment of the heart disorder.

In some embodiments of any of the methods described herein, the method includes identifying one or more biomarker(s) showing elevated level(s) in the location in the sample as compared to reference level(s) of the one or more biomarker(s) in a corresponding location in a sample comprising heart tissue obtained from a control animal as candidate biomarker(s) for determining efficacy of a treatment of the heart disorder. In some embodiments of any of the methods described herein, the method further includes performing an experiment to validate whether the one or more identified candidate biomarker(s) provides for an accurate assessment of the efficacy of a treatment of the heart disease or disorder in an animal.

In some embodiments of any of the methods described herein, the method includes identifying one or more biomarker(s) showing decreased level(s) in the location in the sample as compared to reference level(s) of the one or more biomarker(s) in a corresponding location in a sample comprising heart tissue obtained from a control animal, as candidate biomarker(s) for determining efficacy of a treatment of a heart disorder. In some embodiments of any of the methods described herein, the method further includes performing an experiment to validate whether the one or more identified candidate biomarker(s) provides for an accurate assessment of the efficacy of a treatment of the heart disorder in an animal.

In some embodiments of any of the methods described herein, the location is in the brachiocephalic trunk, left common carotid artery, left subclavian artery, aortic arch, aorta, superior vena cava, right pulmonary artery, left pulmonary artery, ligamentum arteriosum, right pulmonary veins, left pulmonary veins, ascending aorta, pulmonary trunk, auricle of left atrium, right atrium, left atrium, circumflex artery, inferior vena cava, right coronary artery, left coronary artery, anterior cardiac vein, coronary sinus, circumflex branch of left coronary artery, right ventricle, left ventricle, small cardiac vein, great cardiac vein, middle cardiac vein, right marginal artery, anterior interventricular artery, or posterior interventricular artery.

In some embodiments of any of the methods described herein, the heart disorder is selected from the group consisting of: congenital heart disease or disorder, arrhythmia, tachycardia, bradycardia, premature ventricular contraction, fibrillation, coronary artery disease (CAD), heart muscle disease, dilated cardiomyopathy, myocardial infarction, heart failure, hypertrophic cardiomyopathy, mitral regurgitation, heart valve disease, mitral valve prolapse, pulmonary stenosis, pericardial disease, heart infection, aneurysm, and sudden cardiac arrest.

In some embodiments of any of the methods described herein, the animal is a mouse, a rat, a rabbit, a cat, a pig, a human, a nonhuman primate, a zebrafish, a naked mole rat, or a dog. In some embodiments of any of the methods described herein, the control animal is a sex-matched, age-matched healthy animal or a population of sex-matched, age-matched healthy animals. In some embodiments of any of the methods described herein, the control animal is a sex-matched, age-matched animal that does not have a heart disorder or is not at risk of having a heart disease or disorder. In some embodiments of any of the methods described herein, the one or more biomarkers is a nucleic acid. In some embodiments of any of the methods described herein, the nucleic acid is DNA. In some embodiments of any of the methods described herein, the nucleic acid is RNA. In some embodiments of any of the methods described herein, the RNA is mRNA.

In some embodiments of any of the methods described herein, step (a) of the method includes: delivering a probe to the sample, where the sample is affixed to a support, and the probe comprises (i) a sequence that binds specifically to one of the one or more biomarkers and (ii) a spatial barcode that identifies the location where the probe interacted with the sample; and determining all or a portion of (i) the spatial barcode or a complement thereof, and (ii) the sequence that binds specifically to one of the biomarkers or a complement thereof, to determine the level of the biomarker in the location in the sample.

In some embodiments of any of the methods described herein, the determining step comprises sequencing. In some embodiments of any of the methods described herein, the sequencing comprises high throughput sequencing. In some embodiments of any of the methods described herein, the one or more biomarkers is a protein, a peptide, or a combination thereof. In some embodiments of any of the methods described herein, step (a) of the method comprises: contacting the sample with a binding agent that specifically binds to one of the one or more biomarker(s), where the binding agent further comprises an oligonucleotide having a sequence; and sequencing all or a portion of the sequence of the oligonucleotide or a complement thereof, from a probe specifically bound to the biomarker in the location of the sample, to determine the level of the biomarker in the location in the sample. In some embodiments of any of the methods described herein, the binding agent includes an antibody or an antigen-binding antibody fragment. In some embodiments of any of the methods described herein, the sequencing comprises high throughput sequencing.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.As used herein, the term “heart disorder” means a disease or disorder that has a detrimental effect on one or more cells within the heart of an animal. In some embodiments, the detrimental effect on one or more cells within the heart of an animal include, e.g., dysfunction of one or more cells, cytotoxicity (e.g., necrosis or apoptosis) in one or more cells, upregulation of stress signaling pathways in one or more cells, unregulated cell growth in one or more cells, misregulated cell signaling pathways, and upregulated inflammation or inflammation signaling pathways in one or more cells. Non-limiting examples of heart disorders are described herein. Additional examples of heart disorders are known in the art.

As used herein, the term “candidate drug target” means a biomarker (e.g., a nucleic acid (e.g., gene or mRNA), protein, or any of the other exemplary biomarkers described herein) that has been identified for further analysis/experimentation as to whether the biomarker will be an effective target for treatment of a heart disorder.

As used herein, the term “diagnostic biomarker” means a biomarker (e.g., a protein and a nucleic acid (e.g., mRNA), or any of the other biomarkers described herein) that can be used alone or in combination with one or more additional biomarkers or other diagnostic tests (e.g., any of the additional biomarkers and other diagnostic tests described herein or known in the art) to diagnose a heart disorder in an animal.

As used herein, the term “candidate prognostic biomarker of a heart disorder” means a biomarker (e.g., a nucleic acid (e.g., gene or mRNA), protein, or any of the other exemplary biomarkers described herein) that has been identified for further analysis/experimentation to determine whether the biomarker will provide for an accurate assessment of prognosis of a heart disorder in an animal.

As used herein, the term “candidate biomarker for determining efficacy of a treatment for a heart disorder” means a biomarker (e.g., a nucleic acid (e.g., gene or mRNA), protein, or any of the other exemplary biomarkers described herein) that has been identified for further analysis/experimentation to determine whether the biomarker will provide for an accurate assessment of efficacy of a treatment for a heart disorder in an animal.

By the phrase “increased likelihood of having a myocardial infarction” means a subject's increased risk of having a myocardial infarction in the future as compared to a control subject population (e.g., a sex-matched, and age-matched control subject population). For example, a control subject population can not have an elevation in the level of one or more biomarkers (as described herein as being indicative of increased risk of having a myocardial infarction) and/or can not have a decrease in the level of one or more biomarkers (as described herein as being indicative of increased risk of having a myocardial infarction). In some embodiments, a control subject population does not have other risk factors for having a myocardial infarction. In some embodiments, a control subject population can have one or more of: absence of genetic mutations that indicate an increased risk of having a myocardial infarction, a familial history of having a myocardial infarction, an absence of other biomarker levels that indicate an increased risk of having a myocardial infarction, and an absence of other behavioral risk factors that indicate an increased risk of having a myocardial infarction. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The patent or application file contains at least one drawing executed in color. Copies of this patent or patent application publication with color drawing(s) will be provided by the Office upon request and payment of the necessary fee.

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce a spatially-barcoded cells or cellular contents.

FIG. 7A shows a haemotoxylin and eosin-stained heart tissue section.

FIG. 7B shows the region of the heart tissue section in FIG. 7A that corresponds to a normal cardiac tissue section (left) and a diseased (myocardial infarction) cardiac tissue section (right). The dotted regions show the overlay of the tissue sections with a spatial array.

### DETAILED DESCRIPTION

### Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product (also referred to herein as a connected probe) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

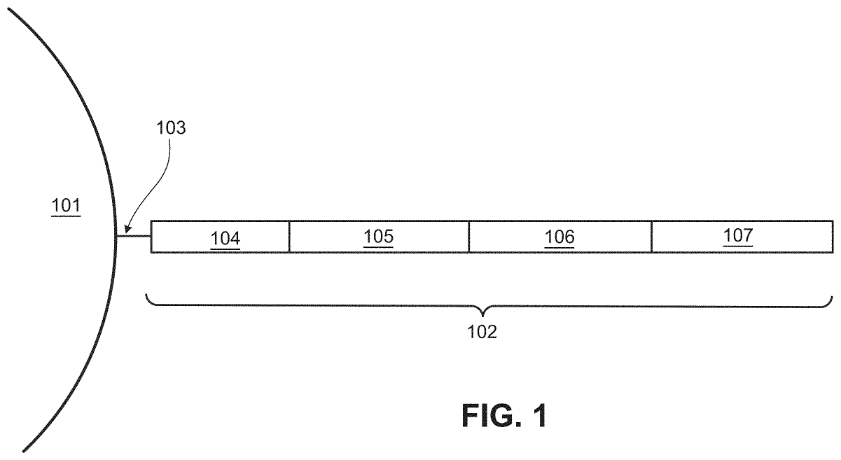

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe (e.g., a ligation product) described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe (e.g., a ligation product) described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

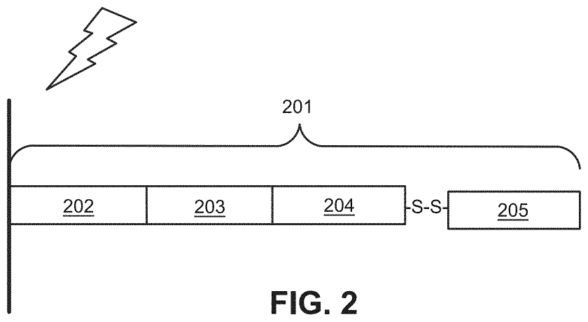

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

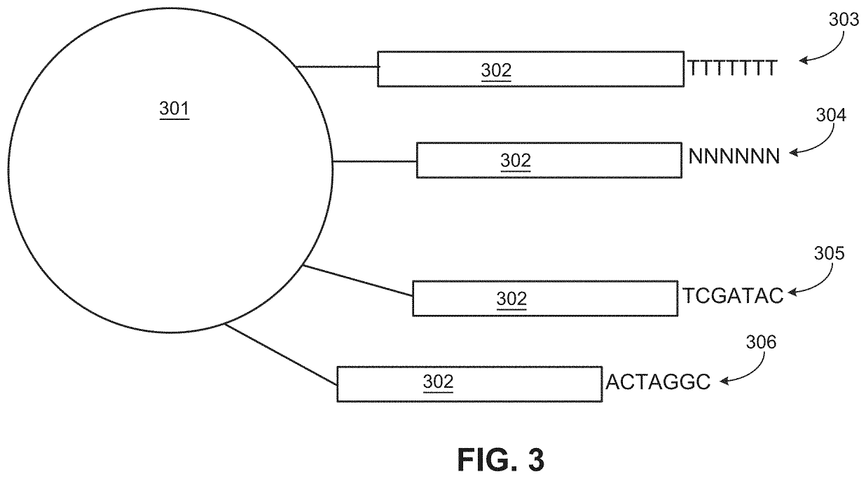

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to the analyte capture agent of interest 305. A fourth type of capture probe associated with the feature includes the spatial barcode 302 in combination with a capture probe that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663. In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe.

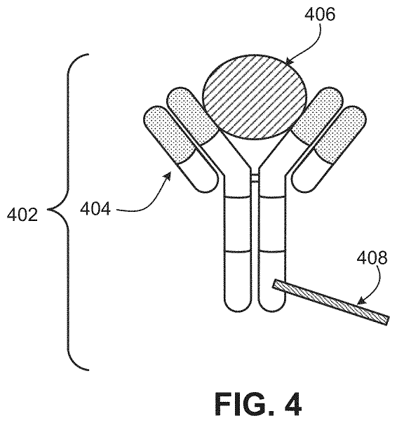

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety 404 can include a polypeptide and/or an aptamer. The analyte-binding moiety 404 can include an antibody or antibody fragment (e.g., an antigen-binding fragment).

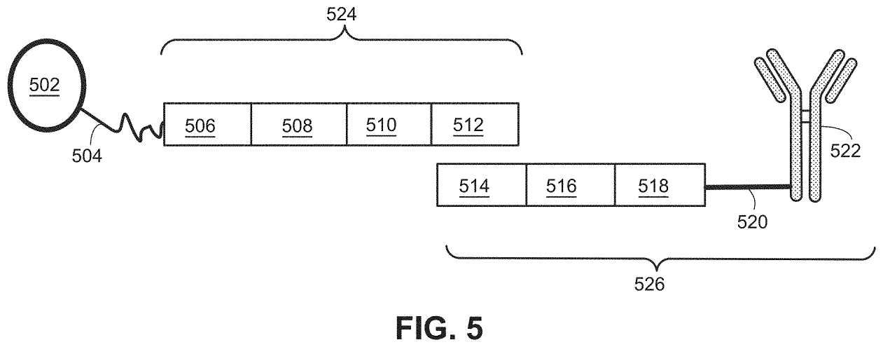

FIG. 5 is a schematic diagram depicting an exemplary interaction between a feature-immobilized capture probe 524 and an analyte capture agent 526. The feature-immobilized capture probe 524 can include a spatial barcode 508 as well as one or more functional sequence 506 and UMI 510, as described elsewhere herein. The capture probe can also include a capture domain 512 that is capable of binding to an analyte capture agent 526. The analyte capture agent 526 can include a functional sequence 518, capture agent barcode domain 516, and an analyte capture sequence (which may also be referred to as a capture handle sequence) 514 that is capable of binding to the capture domain 512 of the capture probe 524. The analyte capture agent can also include a linker 520 that allows the capture agent barcode domain 516 to couple to the analyte binding moiety 522.

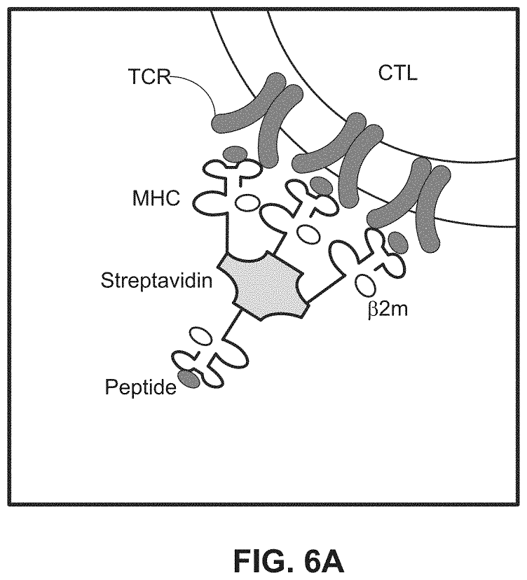

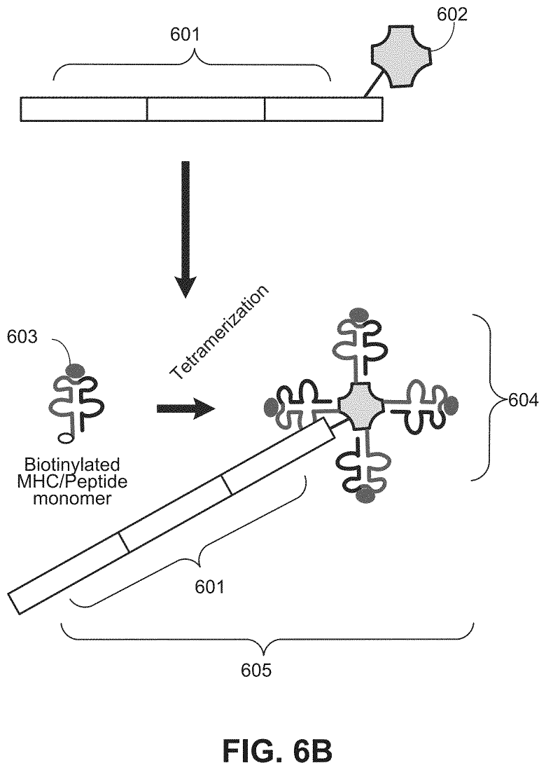

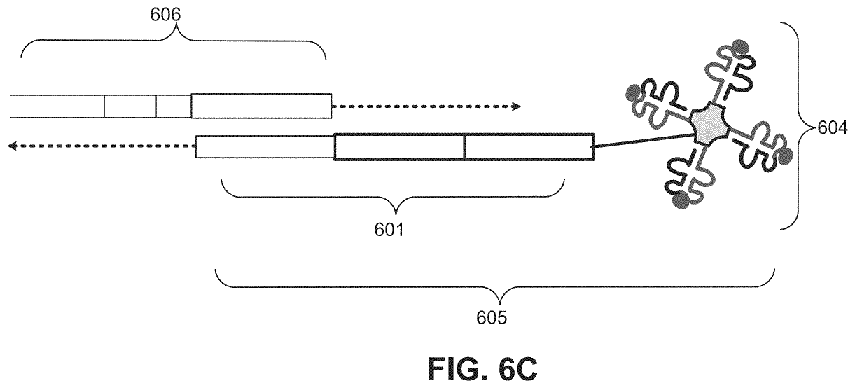

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 1105. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligation products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product (also referred to herein as a connected probe). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., an RNAse, e.g., RNase A, RNase C, RNase H, or RNase I). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Also provided herein are methods for identifying a candidate drug target for treatment of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as candidate drug target(s) for treatment of the heart disorder.

Also provided herein are methods for identifying a diagnostic biomarker of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as diagnostic biomarker(s) of the heart disorder.

Also provided herein are methods for identifying a candidate prognostic biomarker of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as candidate prognostic biomarker(s) of the heart disorder.

Also provided herein are methods for determining a candidate biomarker for determining efficacy of a treatment of a heart disorder that include: (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) in a different location within the sample or in a corresponding location in a sample comprising heart tissue obtained from a control animal, and/or (ii) one or more biomarker(s) showing decreased level(s) in the location in the sample as compared to reference level(s) of the one or more biomarker(s) in a different location within the sample or in a corresponding location in a sample comprising heart tissue obtained from a control animal, as a candidate biomarker for determining efficacy of a treatment of the heart disorder.

Also provided herein are methods of diagnosing a subject as having myocardial infarction that include: (a) determining a level of one or more of: (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; (18) ribosomal protein L38 (RPL38), or a byproduct or precursor or degradation product thereof; (19) ribosomal protein S21 (RPS21), or a byproduct or precursor or degradation product thereof; (20) ribosomal protein S10 (RPS10), or a byproduct or precursor or degradation product thereof; (21) ribonuclease A family member 1, pancreatic (RNASE1), or a byproduct or precursor or degradation product thereof; (22) ribosomal protein S25 (RPS25), or a byproduct or precursor or degradation product thereof; (23) ribosomal protein L26 (RPL26), or a byproduct or precursor or degradation product thereof; (24) ribosomal protein L37a (RPL37A), or a byproduct or precursor or degradation product thereof; (25) ribosomal protein S15a (RPS15A), or a byproduct or precursor or degradation product thereof; (26) eukaryotic translation elongation factor 1 alpha 2 (EEF1A2), or a byproduct or precursor or degradation product thereof; (27) ribosomal protein S27 (RPS27), or a byproduct or precursor or degradation product thereof; (28) nexilin F-actin binding protein (NEXN), or a byproduct or precursor or degradation product thereof; (29) collagen type I alpha 1 chain (COL1A1), or a byproduct or precursor or degradation product thereof; (30) ribosomal protein L23 (RPL23), or a byproduct or precursor or degradation product thereof; (31) collagen type III alpha 1 chain (COL3A1), or a byproduct or precursor or degradation product thereof; (32) ATP synthase F1 subunit epsilon (ATP5F1E), or a byproduct or precursor or degradation product thereof; (33) ribosomal protein S8 (RPS8), or a byproduct or precursor or degradation product thereof; (34) ribosomal protein L31 (RPL31), or a byproduct or precursor or degradation product thereof; (35) poly(A) binding protein cytoplasmic 1 (PABPC1), or a byproduct or precursor or degradation product thereof; (36) ribosomal protein S28 (RPS28), or a byproduct or precursor or degradation product thereof; (37) fatty acid binding protein 4 (FABP4), or a byproduct or precursor or degradation product thereof; (38) decorin (DCN), or a byproduct or precursor or degradation product thereof; (39) matrix Gla protein (MGP), or a byproduct or precursor or degradation product thereof; (40) ribosomal protein L22 (RPL22), or a byproduct or precursor or degradation product thereof; (41) ribosomal protein L39 (RPL39), or a byproduct or precursor or degradation product thereof; (42) complement C1q A chain (C1QA), or a byproduct or precursor or degradation product thereof; (43) complement C1q B chain (C1QB), or a byproduct or precursor or degradation product thereof; (44) myosin heavy chain 6 (MYH6), or a byproduct or precursor or degradation product thereof; (45) secreted protein acidic and cysteine rich (SPARC), or a byproduct or precursor or degradation product thereof; (46) translation machinery associated 7 homolog (TMA7), or a byproduct or precursor or degradation product thereof; (47) ribosomal protein L23a (RPL23A), or a byproduct or precursor or degradation product thereof; (48) NADH:ubiquinone oxidoreductase subunit A1 (NDUFA1), or a byproduct or precursor or degradation product thereof; (49) cytochrome c oxidase subunit 7C (COX7C), or a byproduct or precursor or degradation product thereof; and (50) ferritin heavy chain 1 (FTH1), or a byproduct or precursor or degradation product thereof, in a biological sample from a subject; and (b) identifying a subject having decreased level(s) of one or more of (1)-(18), in the biological sample as compared to reference level(s) of the one or more of (1)-(18), and/or identifying a subject having increased level(s) of one or more of (19) through (50) in the biological sample as compared to reference level(s) of the one or more of (19) through (50), as having myocardial infarction.

Also provided herein are methods of identifying a subject as having an increased likelihood of having a myocardial infarction that include: (a) determining a level of one or more of (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; (18) ribosomal protein L38 (RPL38), or a byproduct or precursor or degradation product thereof; (19) ribosomal protein S21 (RPS21), or a byproduct or precursor or degradation product thereof; (20) ribosomal protein S10 (RPS10), or a byproduct or precursor or degradation product thereof; (21) ribonuclease A family member 1, pancreatic (RNASE1), or a byproduct or precursor or degradation product thereof; (22) ribosomal protein S25 (RPS25), or a byproduct or precursor or degradation product thereof; (23) ribosomal protein L26 (RPL26), or a byproduct or precursor or degradation product thereof; (24) ribosomal protein L37a (RPL37A), or a byproduct or precursor or degradation product thereof; (25) ribosomal protein S15a (RPS15A), or a byproduct or precursor or degradation product thereof; (26) eukaryotic translation elongation factor 1 alpha 2 (EEF1A2), or a byproduct or precursor or degradation product thereof; (27) ribosomal protein S27 (RPS27), or a byproduct or precursor or degradation product thereof; (28) nexilin F-actin binding protein (NEXN), or a byproduct or precursor or degradation product thereof; (29) collagen type I alpha 1 chain (COL1A1), or a byproduct or precursor or degradation product thereof; (30) ribosomal protein L23 (RPL23), or a byproduct or precursor or degradation product thereof; (31) collagen type III alpha 1 chain (COL3A1), or a byproduct or precursor or degradation product thereof; (32) ATP synthase F1 subunit epsilon (ATP5F1E), or a byproduct or precursor or degradation product thereof; (33) ribosomal protein S8 (RPS8), or a byproduct or precursor or degradation product thereof; (34) ribosomal protein L31 (RPL31), or a byproduct or precursor or degradation product thereof; (35) poly(A) binding protein cytoplasmic 1 (PABPC1), or a byproduct or precursor or degradation product thereof; (36) ribosomal protein S28 (RPS28), or a byproduct or precursor or degradation product thereof; (37) fatty acid binding protein 4 (FABP4), or a byproduct or precursor or degradation product thereof; (38) decorin (DCN), or a byproduct or precursor or degradation product thereof; (39) matrix Gla protein (MGP), or a byproduct or precursor or degradation product thereof; (40) ribosomal protein L22 (RPL22), or a byproduct or precursor or degradation product thereof; (41) ribosomal protein L39 (RPL39), or a byproduct or precursor or degradation product thereof; (42) complement C1q A chain (C1QA), or a byproduct or precursor or degradation product thereof; (43) complement C1q B chain (C1QB), or a byproduct or precursor or degradation product thereof; (44) myosin heavy chain 6 (MYH6), or a byproduct or precursor or degradation product thereof; (45) secreted protein acidic and cysteine rich (SPARC), or a byproduct or precursor or degradation product thereof; (46) translation machinery associated 7 homolog (TMA7), or a byproduct or precursor or degradation product thereof; (47) ribosomal protein L23a (RPL23A), or a byproduct or precursor or degradation product thereof; (48) NADH:ubiquinone oxidoreductase subunit A1 (NDUFA1), or a byproduct or precursor or degradation product thereof; (49) cytochrome c oxidase subunit 7C (COX7C), or a byproduct or precursor or degradation product thereof; and (50) ferritin heavy chain 1 (FTH1), or a byproduct or precursor or degradation product thereof, in a biological sample from a subject; and (b) identifying a subject having decreased level(s) of one or more of (1)-(18), in the biological sample as compared to reference level(s) of the one or more of (1)-(18), and/or identifying a subject having increased level(s) of one or more of (19) through (50) in the biological sample as compared to reference level(s) of the one or more of (19) through (50), as having an increased likelihood of having a myocardial infarction.

Also provided herein are methods of monitoring risk of having a myocardial infarction in a subject over time that include: (a) determining a first level of one or more of: (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; (18) ribosomal protein L38 (RPL38), or a byproduct or precursor or degradation product thereof; (19) ribosomal protein S21 (RPS21), or a byproduct or precursor or degradation product thereof; (20) ribosomal protein S10 (RPS10), or a byproduct or precursor or degradation product thereof; (21) ribonuclease A family member 1, pancreatic (RNASE1), or a byproduct or precursor or degradation product thereof; (22) ribosomal protein S25 (RPS25), or a byproduct or precursor or degradation product thereof; (23) ribosomal protein L26 (RPL26), or a byproduct or precursor or degradation product thereof; (24) ribosomal protein L37a (RPL37A), or a byproduct or precursor or degradation product thereof; (25) ribosomal protein S15a (RPS15A), or a byproduct or precursor or degradation product thereof; (26) eukaryotic translation elongation factor 1 alpha 2 (EEF1A2), or a byproduct or precursor or degradation product thereof; (27) ribosomal protein S27 (RPS27), or a byproduct or precursor or degradation product thereof; (28) nexilin F-actin binding protein (NEXN), or a byproduct or precursor or degradation product thereof; (29) collagen type I alpha 1 chain (COL1A1), or a byproduct or precursor or degradation product thereof; (30) ribosomal protein L23 (RPL23), or a byproduct or precursor or degradation product thereof; (31) collagen type III alpha 1 chain (COL3A1), or a byproduct or precursor or degradation product thereof; (32) ATP synthase F1 subunit epsilon (ATP5F1E), or a byproduct or precursor or degradation product thereof; (33) ribosomal protein S8 (RPS8), or a byproduct or precursor or degradation product thereof; (34) ribosomal protein L31 (RPL31), or a byproduct or precursor or degradation product thereof; (35) poly(A) binding protein cytoplasmic 1 (PABPC1), or a byproduct or precursor or degradation product thereof; (36) ribosomal protein S28 (RPS28), or a byproduct or precursor or degradation product thereof; (37) fatty acid binding protein 4 (FABP4), or a byproduct or precursor or degradation product thereof; (38) decorin (DCN), or a byproduct or precursor or degradation product thereof; (39) matrix Gla protein (MGP), or a byproduct or precursor or degradation product thereof; (40) ribosomal protein L22 (RPL22), or a byproduct or precursor or degradation product thereof; (41) ribosomal protein L39 (RPL39), or a byproduct or precursor or degradation product thereof; (42) complement C1q A chain (C1QA), or a byproduct or precursor or degradation product thereof; (43) complement C1q B chain (C1QB), or a byproduct or precursor or degradation product thereof; (44) myosin heavy chain 6 (MYH6), or a byproduct or precursor or degradation product thereof; (45) secreted protein acidic and cysteine rich (SPARC), or a byproduct or precursor or degradation product thereof; (46) translation machinery associated 7 homolog (TMA7), or a byproduct or precursor or degradation product thereof; (47) ribosomal protein L23a (RPL23A), or a byproduct or precursor or degradation product thereof; (48) NADH:ubiquinone oxidoreductase subunit A1 (NDUFA1), or a byproduct or precursor or degradation product thereof; (49) cytochrome c oxidase subunit 7C (COX7C), or a byproduct or precursor or degradation product thereof; and (50) ferritin heavy chain 1 (FTH1), or a byproduct or precursor or degradation product thereof, in a first biological sample obtained from a subject at a first time point; (b) determining a second level of one or more of (1)-(50), in a second biological sample obtained from the subject at a second time point; (c) identifying: (i) a subject having increased second level(s) of one or more of (19) through (50) as compared to the first level(s) of the one or more of (19)-(50), and/or decreased second level(s) of one or more of (1) through (18) as compared to the first level(s) of the one or more of (1) through (18), as having an increasing risk of having a myocardial infarction, or (ii) a subject having about the same or decreased second level(s) of one or more of (19) through (50) as compared to the first level(s) of the one or more of (19) through (50), and/or about the same or increased second level(s) of one or more of (1) through (18) as compared to the first level(s) of the one or more of (1) through (18), as having about the same or a decreasing risk of having a myocardial infarction.

Also provided herein are methods of determining efficacy of a treatment for reducing the risk of having a myocardial infarction in a subject that include: (a) determining a first level of one or more of: (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; (18) ribosomal protein L38 (RPL38), or a byproduct or precursor or degradation product thereof; (19) ribosomal protein S21 (RPS21), or a byproduct or precursor or degradation product thereof; (20) ribosomal protein S10 (RPS10), or a byproduct or precursor or degradation product thereof; (21) ribonuclease A family member 1, pancreatic (RNASE1), or a byproduct or precursor or degradation product thereof; (22) ribosomal protein S25 (RPS25), or a byproduct or precursor or degradation product thereof; (23) ribosomal protein L26 (RPL26), or a byproduct or precursor or degradation product thereof; (24) ribosomal protein L37a (RPL37A), or a byproduct or precursor or degradation product thereof; (25) ribosomal protein 515a (RPS15A), or a byproduct or precursor or degradation product thereof; (26) eukaryotic translation elongation factor 1 alpha 2 (EEF1A2), or a byproduct or precursor or degradation product thereof; (27) ribosomal protein S27 (RPS27), or a byproduct or precursor or degradation product thereof; (28) nexilin F-actin binding protein (NEXN), or a byproduct or precursor or degradation product thereof; (29) collagen type I alpha 1 chain (COL1A1), or a byproduct or precursor or degradation product thereof; (30) ribosomal protein L23 (RPL23), or a byproduct or precursor or degradation product thereof; (31) collagen type III alpha 1 chain (COL3A1), or a byproduct or precursor or degradation product thereof; (32) ATP synthase F1 subunit epsilon (ATP5F1E), or a byproduct or precursor or degradation product thereof; (33) ribosomal protein S8 (RPS8), or a byproduct or precursor or degradation product thereof; (34) ribosomal protein L31 (RPL31), or a byproduct or precursor or degradation product thereof; (35) poly(A) binding protein cytoplasmic 1 (PABPC1), or a byproduct or precursor or degradation product thereof; (36) ribosomal protein S28 (RPS28), or a byproduct or precursor or degradation product thereof; (37) fatty acid binding protein 4 (FABP4), or a byproduct or precursor or degradation product thereof; (38) decorin (DCN), or a byproduct or precursor or degradation product thereof; (39) matrix Gla protein (MGP), or a byproduct or precursor or degradation product thereof; (40) ribosomal protein L22 (RPL22), or a byproduct or precursor or degradation product thereof; (41) ribosomal protein L39 (RPL39), or a byproduct or precursor or degradation product thereof; (42) complement C1q A chain (C1QA), or a byproduct or precursor or degradation product thereof; (43) complement C1q B chain (C1QB), or a byproduct or precursor or degradation product thereof; (44) myosin heavy chain 6 (MYH6), or a byproduct or precursor or degradation product thereof; (45) secreted protein acidic and cysteine rich (SPARC), or a byproduct or precursor or degradation product thereof; (46) translation machinery associated 7 homolog (TMA7), or a byproduct or precursor or degradation product thereof; (47) ribosomal protein L23a (RPL23A), or a byproduct or precursor or degradation product thereof; (48) NADH:ubiquinone oxidoreductase subunit A1 (NDUFA1), or a byproduct or precursor or degradation product thereof; (49) cytochrome c oxidase subunit 7C (COX7C), or a byproduct or precursor or degradation product thereof; and (50) ferritin heavy chain 1 (FTH1), or a byproduct or precursor or degradation product thereof, in a first biological sample obtained from a subject at a first time point; (b) determining a second level of the one or more of (1) through (50), in a second biological sample obtained from the subject at a second time point, wherein the subject is administered one or more doses of a treatment for reducing the risk of having a myocardial infarction between the first and second time points; and (c) identifying: (i) the treatment as being effective in a subject having about the same or decreased second level(s) of one or more of (19) through (50), as compared to the first level(s) of the one or more of (19) through (50), and/or having an increased second level(s) of one or more of (1) through (18) as compared to the first level(s) of the one or more of (1) through (18); or (ii) the treatment as not being effective in a subject having increased second level(s) of one or more of (19) through (50), as compared to the first level(s) of the one or more of (19) through (50), and/or having about the same or decreased second level(s) of one or more of (1) through (18) as compared to the first level(s) of the one or more of (1) through (18).

Also provided herein are kits that include: (a) one or more of: (1) an antibody that binds specifically to ribosome protein L17 (RPL17), or a byproduct or precursor or degradation product thereof; (2) an antibody that binds specifically to ribosomal protein L36a (RPL36A), or a byproduct or precursor or degradation product thereof; (3) an antibody that binds specifically to thymosin beta 4 X-linked (TMSB4X), or a byproduct or precursor or degradation product thereof; (4) an antibody that binds specifically to coiled-coil domain containing 80 (CCDC80), or a byproduct or precursor or degradation product thereof; (5) an antibody that binds specifically to ferritin light chain (FTL), or a byproduct or precursor or degradation product thereof; (6) an antibody that binds specifically to ribosomal protein L37 (RPL37), or a byproduct or precursor or degradation product thereof; (7) an antibody that binds specifically to heme oxygenase 1 (HMOX1), or a byproduct or precursor or degradation product thereof; (8) an antibody that binds specifically to Y-box binding protein 1 (YBX1), or a byproduct or precursor or degradation product thereof; (9) an antibody that binds specifically to ribosomal protein S17 (RPS17), or a byproduct or precursor or degradation product thereof; (10) an antibody that binds specifically to ribosomal protein S29 (RPS29), or a byproduct or precursor or degradation product thereof; (11) an antibody that binds specifically to pancreatic progenitor cell differentiation and proliferation factor (PPDPF), or a byproduct or precursor or degradation product thereof; (12) an antibody that binds specifically to Y-box binding protein 3 (YBX3), or a byproduct or precursor or degradation product thereof; (13) an antibody that binds specifically to vimentin (VIM), or a byproduct or precursor or degradation product thereof; (14) an antibody that binds specifically to NADH:ubiquinone oxidoreductase subunit B1 (NDUFB1), or a byproduct or precursor or degradation product thereof; (15) an antibody that binds specifically to ribosomal protein L34 (RPL34), or a byproduct or precursor or degradation product thereof; (16) an antibody that binds specifically to macrophage migration inhibitory factor (MIF), or a byproduct or precursor or degradation product thereof; (17) an antibody that binds specifically to phospholipid transfer protein (PLTP), or a byproduct or precursor or degradation product thereof; (18) an antibody that binds specifically to ribosomal protein L38 (RPL38), or a byproduct or precursor or degradation product thereof; (19) an antibody that binds specifically to ribosomal protein S21 (RPS21), or a byproduct or precursor or degradation product thereof; (20) an antibody that binds specifically to ribosomal protein S10 (RPS10), or a byproduct or precursor or degradation product thereof; (21) an antibody that binds specifically to ribonuclease A family member 1, pancreatic (RNASE1), or a byproduct or precursor or degradation product thereof; (22) an antibody that binds specifically to ribosomal protein S25 (RPS25), or a byproduct or precursor or degradation product thereof; (23) an antibody that binds specifically to ribosomal protein L26 (RPL26), or a byproduct or precursor or degradation product thereof; (24) an antibody that binds specifically to ribosomal protein L37a (RPL37A), or a byproduct or precursor or degradation product thereof; (25) an antibody that binds specifically to ribosomal protein S15a (RPS15A), or a byproduct or precursor or degradation product thereof; (26) an antibody that binds specifically to eukaryotic translation elongation factor 1 alpha 2 (EEF1A2), or a byproduct or precursor or degradation product thereof; (27) an antibody that binds specifically to ribosomal protein S27 (RPS27), or a byproduct or precursor or degradation product thereof; (28) an antibody that binds specifically to nexilin F-actin binding protein (NEXN), or a byproduct or precursor or degradation product thereof; (29) an antibody that binds specifically to collagen type I alpha 1 chain (COL1A1), or a byproduct or precursor or degradation product thereof; (30) an antibody that binds specifically to ribosomal protein L23 (RPL23), or a byproduct or precursor or degradation product thereof; (31) an antibody that binds specifically to collagen type III alpha 1 chain (COL3A1), or a byproduct or precursor or degradation product thereof; (32) an antibody that binds specifically to ATP synthase F1 subunit epsilon (ATP5F1E), or a byproduct or precursor or degradation product thereof; (33) an antibody that binds specifically to ribosomal protein S8 (RPS8), or a byproduct or precursor or degradation product thereof; (34) an antibody that binds specifically to ribosomal protein L31 (RPL31), or a byproduct or precursor or degradation product thereof; (35) an antibody that binds specifically to poly(A) binding protein cytoplasmic 1 (PABPC1), or a byproduct or precursor or degradation product thereof; (36) an antibody that binds specifically to ribosomal protein S28 (RPS28), or a byproduct or precursor or degradation product thereof; (37) an antibody that binds specifically to fatty acid binding protein 4 (FABP4), or a byproduct or precursor or degradation product thereof; (38) an antibody that binds specifically to decorin (DCN), or a byproduct or precursor or degradation product thereof; (39) an antibody that binds specifically to matrix Gla protein (MGP), or a byproduct or precursor or degradation product thereof; (40) an antibody that binds specifically to ribosomal protein L22 (RPL22), or a byproduct or precursor or degradation product thereof; (41) an antibody that binds specifically to ribosomal protein L39 (RPL39), or a byproduct or precursor or degradation product thereof; (42) an antibody that binds specifically to complement C1q A chain (C1QA), or a byproduct or precursor or degradation product thereof; (43) an antibody that binds specifically to complement C1q B chain (C1QB), or a byproduct or precursor or degradation product thereof; (44) an antibody that binds specifically to myosin heavy chain 6 (MYH6), or a byproduct or precursor or degradation product thereof; (45) an antibody that binds specifically to secreted protein acidic and cysteine rich (SPARC), or a byproduct or precursor or degradation product thereof; (46) an antibody that binds specifically to translation machinery associated 7 homolog (TMA7), or a byproduct or precursor or degradation product thereof; (47) an antibody that binds specifically to ribosomal protein L23a (RPL23A), or a byproduct or precursor or degradation product thereof; (48) an antibody that binds specifically to NADH:ubiquinone oxidoreductase subunit A1 (NDUFA1), or a byproduct or precursor or degradation product thereof; (49) an antibody that binds specifically to cytochrome c oxidase subunit 7C (COX7C), or a byproduct or precursor or degradation product thereof; and (50) an antibody that binds specifically to ferritin heavy chain 1 (FTH1), or a byproduct or precursor or degradation product thereof; and (b) instructions for performing any of the methods described herein.

Also provided herein are methods of identifying a patient subpopulation for which a treatment for reducing the risk of having a myocardial infarction is effective that include: (a) administering a treatment for reducing the risk of having a myocardial infarction to a patient subpopulation; (b) determining (i) a first level of one or more of: (1) ribosomal protein S21 (RPS21), or a byproduct or precursor or degradation product thereof; (2) ribosomal protein S10 (RPS10), or a byproduct or precursor or degradation product thereof; (3) ribonuclease A family member 1, pancreatic (RNASE1), or a byproduct or precursor or degradation product thereof; (4) ribosomal protein S25 (RPS25), or a byproduct or precursor or degradation product thereof; (5) ribosomal protein L26 (RPL26), or a byproduct or precursor or degradation product thereof; (6) ribosomal protein L37a (RPL37A), or a byproduct or precursor or degradation product thereof; (7) ribosomal protein S15a (RPS15A), or a byproduct or precursor or degradation product thereof; (8) eukaryotic translation elongation factor 1 alpha 2 (EEF1A2), or a byproduct or precursor or degradation product thereof; (9) ribosomal protein S27 (RPS27), or a byproduct or precursor or degradation product thereof; (10) nexilin F-actin binding protein (NEXN), or a byproduct or precursor or degradation product thereof; (11) collagen type I alpha 1 chain (COL1A1), or a byproduct or precursor or degradation product thereof; (12) ribosomal protein L23 (RPL23), or a byproduct or precursor or degradation product thereof; (13) collagen type III alpha 1 chain (COL3A1), or a byproduct or precursor or degradation product thereof; (14) ATP synthase F1 subunit epsilon (ATP5F1E), or a byproduct or precursor or degradation product thereof; (15) ribosomal protein S8 (RPS8), or a byproduct or precursor or degradation product thereof; (16) ribosomal protein L31 (RPL31), or a byproduct or precursor or degradation product thereof; (17) poly(A) binding protein cytoplasmic 1 (PABPC1), or a byproduct or precursor or degradation product thereof; (18) ribosomal protein S28 (RPS28), or a byproduct or precursor or degradation product thereof; (19) fatty acid binding protein 4 (FABP4), or a byproduct or precursor or degradation product thereof; (20) decorin (DCN), or a byproduct or precursor or degradation product thereof; (21) matrix Gla protein (MGP), or a byproduct or precursor or degradation product thereof; (22) ribosomal protein L22 (RPL22), or a byproduct or precursor or degradation product thereof; (23) ribosomal protein L39 (RPL39), or a byproduct or precursor or degradation product thereof; (24) complement C1q A chain (C1QA), or a byproduct or precursor or degradation product thereof; (25) complement C1qB chain (C1QB), or a byproduct or precursor or degradation product thereof; (26) myosin heavy chain 6 (MYH6), or a byproduct or precursor or degradation product thereof; (27) secreted protein acidic and cysteine rich (SPARC), or a byproduct or precursor or degradation product thereof; (28) translation machinery associated 7 homolog (TMA7), or a byproduct or precursor or degradation product thereof; (29) ribosomal protein L23a (RPL23A), or a byproduct or precursor or degradation product thereof; (30) NADH:ubiquinone oxidoreductase subunit A1 (NDUFA1), or a byproduct or precursor or degradation product thereof; (31) cytochrome c oxidase subunit 7C (COX7C), or a byproduct or precursor or degradation product thereof; and (32) ferritin heavy chain 1 (FTH1), or a byproduct or precursor or degradation product thereof, in a first biological sample obtained from a patient subpopulation at a first time point, and (ii) second level(s) of the one or more of (1) through (32), in a second biological sample obtained from the patient population at a second time point, wherein the patient subpopulation is administered one or more doses of a treatment for reducing the risk of having a myocardial infarction between the first and second time points; and (c) determining a correlation between efficacy of the treatment for reducing the risk of having a myocardial infarction and the second level(s) from the patient subpopulation as compared to level(s) in a sample obtained from an untreated patient, wherein lower second level(s) in the samples from the patient subpopulation as compared to the level(s) in the sample from the untreated patient is indicative that the treatment is effective for reducing risk of having a myocardial infarction in the patient subpopulation.

Also provided herein are methods of identifying a patient subpopulation for which a treatment for reducing the risk of having a myocardial infarction is effective that include: (a) administering a treatment for reducing the risk of having a myocardial infarction to a patient subpopulation; (b) determining (i) a first level of one or more of: (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; (18) ribosomal protein L38 (RPL38), or a byproduct or precursor or degradation product thereof, in a first biological sample obtained from a patient subpopulation at a first time point, and (ii) second level(s) of the one or more of (1) through (18), in a second biological sample obtained from the patient population at a second time point, wherein the patient subpopulation is administered one or more doses of a treatment for reducing the risk of having a myocardial infarction between the first and second time points; and (c) determining a correlation between efficacy of the treatment for reducing the risk of having a myocardial infarction and the second level(s) from the patient subpopulation as compared to level(s) in a sample obtained from an untreated patient, wherein elevated second level(s) in the samples from the patient subpopulation as compared to the level(s) in the sample from the untreated patient is indicative that the treatment is effective for reducing risk of having a myocardial infarction in the patient subpopulation.

Also provided herein are methods of modifying treatment for reducing the risk of having a myocardial infarction in a subject that include: (a) administering a treatment for reducing the risk of having a myocardial infarction to a subject; (b) determining (i) pre-treatment level(s) of one or more of: (1) ribosomal protein S21 (RPS21), or a byproduct or precursor or degradation product thereof; (2) ribosomal protein S10 (RPS10), or a byproduct or precursor or degradation product thereof; (3) ribonuclease A family member 1, pancreatic (RNASE1), or a byproduct or precursor or degradation product thereof; (4) ribosomal protein S25 (RPS25), or a byproduct or precursor or degradation product thereof; (5) ribosomal protein L26 (RPL26), or a byproduct or precursor or degradation product thereof; (6) ribosomal protein L37a (RPL37A), or a byproduct or precursor or degradation product thereof; (7) ribosomal protein S15a (RPS15A), or a byproduct or precursor or degradation product thereof; (8) eukaryotic translation elongation factor 1 alpha 2 (EEF1A2), or a byproduct or precursor or degradation product thereof; (9) ribosomal protein S27 (RPS27), or a byproduct or precursor or degradation product thereof; (10) nexilin F-actin binding protein (NEXN), or a byproduct or precursor or degradation product thereof; (11) collagen type I alpha 1 chain (COL1A1), or a byproduct or precursor or degradation product thereof; (12) ribosomal protein L23 (RPL23), or a byproduct or precursor or degradation product thereof; (13) collagen type III alpha 1 chain (COL3A1), or a byproduct or precursor or degradation product thereof; (14) ATP synthase F1 subunit epsilon (ATP5F1E), or a byproduct or precursor or degradation product thereof; (15) ribosomal protein S8 (RPS8), or a byproduct or precursor or degradation product thereof; (16) ribosomal protein L31 (RPL31), or a byproduct or precursor or degradation product thereof; (17) poly(A) binding protein cytoplasmic 1 (PABPC1), or a byproduct or precursor or degradation product thereof; (18) ribosomal protein S28 (RPS28), or a byproduct or precursor or degradation product thereof; (19) fatty acid binding protein 4 (FABP4), or a byproduct or precursor or degradation product thereof; (20) decorin (DCN), or a byproduct or precursor or degradation product thereof; (21) matrix Gla protein (MGP), or a byproduct or precursor or degradation product thereof; (22) ribosomal protein L22 (RPL22), or a byproduct or precursor or degradation product thereof; (23) ribosomal protein L39 (RPL39), or a byproduct or precursor or degradation product thereof; (24) complement C1q A chain (C1QA), or a byproduct or precursor or degradation product thereof; (25) complement C1q B chain (C1QB), or a byproduct or precursor or degradation product thereof; (26) myosin heavy chain 6 (MYH6), or a byproduct or precursor or degradation product thereof; (27) secreted protein acidic and cysteine rich (SPARC), or a byproduct or precursor or degradation product thereof; (28) translation machinery associated 7 homolog (TMA7), or a byproduct or precursor or degradation product thereof; (29) ribosomal protein L23a (RPL23A), or a byproduct or precursor or degradation product thereof; (30) NADH:ubiquinone oxidoreductase subunit A1 (NDUFA1), or a byproduct or precursor or degradation product thereof; (31) cytochrome c oxidase subunit 7C (COX7C), or a byproduct or precursor or degradation product thereof; and (32) ferritin heavy chain 1 (FTH1), or a byproduct or precursor or degradation product thereof, in a pre-treatment sample obtained from a patient before treatment and (ii) post-treatment level(s) of the one or more of (1) through (32), in a post-treatment sample obtained from the patient after treatment, wherein higher level(s) of (1) through (32), in the post-treatment sample, as compared to the level(s) of one or more of (1) through (32), in a pre-treatment sample, is indicative of the responsiveness to treatment with the treatment for reducing the risk of having a myocardial infarction; and (c) increasing the amount of the treatment for reducing the risk of having a myocardial infarction administered to the patient based on the higher level(s) of the one or more of (1) through (32) in the post-treatment sample as compared to the level(s) of one or more of (1) through (32) in the pre-treatment sample.

Also provided herein are methods of modifying treatment for reducing the risk of having a myocardial infarction in a subject that include: (a) administering a treatment for reducing the risk of having a myocardial infarction to a subject; (b) determining (i) pre-treatment level(s) of one or more of: (1) ribosome protein L17 (RPL17), or a byproduct or precursor or degradation product thereof; (2) ribosomal protein L36a (RPL36A), or a byproduct or precursor or degradation product thereof; (3) thymosin beta 4 X-linked (TMSB4X), or a byproduct or precursor or degradation product thereof; (4) coiled-coil domain containing 80 (CCDC80), or a byproduct or precursor or degradation product thereof; (5) ferritin light chain (FTL), or a byproduct or precursor or degradation product thereof; (6) ribosomal protein L37 (RPL37), or a byproduct or precursor or degradation product thereof; (7) heme oxygenase 1 (HMOX1), or a byproduct or precursor or degradation product thereof; (8) Y-box binding protein 1 (YBX1), or a byproduct or precursor or degradation product thereof; (9) ribosomal protein S17 (RPS17), or a byproduct or precursor or degradation product thereof; (10) ribosomal protein S29 (RPS29), or a byproduct or precursor or degradation product thereof; (11) pancreatic progenitor cell differentiation and proliferation factor (PPDPF), or a byproduct or precursor or degradation product thereof; (12) Y-box binding protein 3 (YBX3), or a byproduct or precursor or degradation product thereof; (13) vimentin (VIM), or a byproduct or precursor or degradation product thereof; (14) NADH:ubiquinone oxidoreductase subunit B1 (NDUFB1), or a byproduct or precursor or degradation product thereof; (15) ribosomal protein L34 (RPL34), or a byproduct or precursor or degradation product thereof; (16) macrophage migration inhibitory factor (MIF), or a byproduct or precursor or degradation product thereof; (17) phospholipid transfer protein (PLTP), or a byproduct or precursor or degradation product thereof; and (18) ribosomal protein L38 (RPL38), or a byproduct or precursor or degradation product thereof, in a pre-treatment sample obtained from a patient before treatment and (ii) post-treatment level(s) of the one or more of (1) through (18), in a post-treatment sample obtained from the patient after treatment, wherein decreased level(s) of (1) through (18), in the post-treatment sample, as compared to the level(s) of one or more of (1) through (18), in a pre-treatment sample, is indicative of the responsiveness to treatment with the treatment for reducing the risk of having a myocardial infarction; and (c) increasing the amount of the treatment for reducing the risk of having a myocardial infarction administered to the patient based on the decreased level(s) of the one or more of (1) through (18) in the post-treatment sample as compared to the level(s) of one or more of (1) through (18) in the pre-treatment sample.

### II. Spatial Analysis and Heart Disorders

Provided herein are methods of identifying and measuring biomarkers that are dysregulated in a heart disorder, e.g., a myocardial infarction. This disclosure includes methods of detecting biomarkers in various locations in a sample and thereby identifying candidate drug targets for treatment of a heart disorder (e.g., myocardial infarction), methods of identifying a candidate biomarker for efficacy of treatment of a heart disorder (e.g., myocardial infarction), methods of diagnosing a heart disorder (e.g., myocardial infarction) in a subject, methods of identifying a subject with increased likelihood of developing a heart disorder (e.g., myocardial infarction), methods of monitoring the progression of a heart disorder (e.g., myocardial infarction) in a subject, and methods of determining the efficacy of a treatment for a heart disorder (e.g., myocardial infarction), methods for identifying a patient subpopulation for which a therapeutic treatment is effective for a heart disorder (e.g., myocardial infarction), and methods of modifying treatment for a patient with a heart disorder (e.g., myocardial infarction). Also provided herein are kits comprising antibodies to the specific candidate biomarkers identified herein.

The biomarkers for myocardial infarction identified herein can be used for diagnostic, prognostic and therapeutic purposes and include but are not limited to one or more analytes selected from ribosome protein L17 (RPL17), ribosomal protein L36a (RPL36A), thymosin beta 4 X-linked (TMSB4X), coiled-coil domain containing 80 (CCDC80), ferritin light chain (FTL), ribosomal protein L37 (RPL37), heme oxygenase 1 (HMOX1), Y-box binding protein 1 (YBX1), ribosomal protein S17 (RPS17), ribosomal protein S29 (RPS29), pancreatic progenitor cell differentiation and proliferation factor (PPDPF), Y-box binding protein 3 (YBX3), vimentin (VIM), NADH:ubiquinone oxidoreductase subunit B1 (NDUFB1), ribosomal protein L34 (RPL34), macrophage migration inhibitory factor (MIF), phospholipid transfer protein (PLTP), ribosomal protein L38 (RPL38), ribosomal protein S21 (RPS21), ribosomal protein S10 (RPS10), ribonuclease A family member 1, pancreatic (RNASE1), ribosomal protein S25 (RPS25), ribosomal protein L26 (RPL26), ribosomal protein L37a (RPL37A), ribosomal protein S15a (RPS15A), eukaryotic translation elongation factor 1 alpha 2 (EEF1A2), ribosomal protein S27 (RPS27), nexilin F-actin binding protein (NEXN), collagen type I alpha 1 chain (COL1A1), ribosomal protein L23 (RPL23), collagen type III alpha 1 chain (COL3A1), ATP synthase F1 subunit epsilon (ATP5F1E), ribosomal protein S8 (RPS8), ribosomal protein L31 (RPL31), poly(A) binding protein cytoplasmic 1 (PABPC1), ribosomal protein S28 (RPS28), fatty acid binding protein 4 (FABP4), decorin (DCN), matrix Gla protein (MGP), ribosomal protein L22 (RPL22), ribosomal protein L39 (RPL39), complement C1q A chain (C1QA), complement C1q B chain (C1QB), myosin heavy chain 6 (MYH6), secreted protein acidic and cysteine rich (SPARC), translation machinery associated 7 homolog (TMA7), ribosomal protein L23a (RPL23A), NADH:ubiquinone oxidoreductase subunit A1 (NDUFA1), cytochrome c oxidase subunit 7C (COX7C), ferritin heavy chain 1 (FTH1), and byproducts, degradation products, or precursors thereof.

(a) Heart Disorders

As used herein, a heart disorder can be any appropriate heart disorder. In some embodiments, a heart disorder is any condition, disorder, or disease that occurs in heart tissue or a related-tissue. In some embodiments, a heart disorder is manifested by one or more physical symptoms. In some embodiments, a heart disorder is not manifested by symptoms that can be detected by a physician during a physical examination. In some embodiments, the heart disorder can be selected from the group of congenital heart disease or disorder, arrhythmia, tachycardia, bradycardia, premature ventricular contraction, fibrillation, coronary artery disease (CAD), heart muscle disease, dilated cardiomyopathy, myocardial infarction, heart failure, hypertrophic cardiomyopathy, mitral regurgitation, heart valve disease, mitral valve prolapse, pulmonary stenosis, pericardial disease, heart infection, aneurysm, and sudden cardiac arrest.

In some embodiments, a heart disorder can be congenital heart disease or disorder. In some embodiments, a heart disorder can be arrhythmia. In some embodiments, a heart disorder can be tachycardia. In some embodiments, a heart disorder can be bradycardia. In some embodiments, a heart disorder can be premature ventricular contraction. In some embodiments, a heart disorder can be fibrillation. In some embodiments, a heart disorder can be coronary artery disease (CAD). In some embodiments, a heart disorder can be heart muscle disease. In some embodiments, a heart disorder can be dilated cardiomyopathy. In some embodiments, a heart disorder can be myocardial infarction. In some embodiments, a heart disorder can be heart failure (e.g., congestive heart failure). In some embodiments, a heart disorder can be hypertrophic cardiomyopathy. In some embodiments, a heart disorder can be mitral regurgitation. In some embodiments, a heart disorder can be heart valve disease. In some embodiments, a heart disorder can be mitral valve prolapse. In some embodiments, a heart disorder can be pulmonary stenosis. In some embodiments, a heart disorder can be pericardial disease. In some embodiments, a heart disorder can be heart infection. In some embodiments, a heart disorder can be an aneurysm. In some embodiments, a heart disorder can be sudden cardiac arrest.

(b) Animals

As used herein, an animal (or subject, patient, and the like) can be any appropriate animal. In some embodiments, the animal is a human. In some embodiments, an animal can be a pig. In some embodiments, an animal can be selected from the group consisting of a zebrafish, a mouse, a rat, a rabbit, a cat, a dog, a naked mole rat, a nonhuman primate, a pig, and a human. In some embodiments, an animal can be a mammal. In some embodiments, a mammal can be selected from a mouse, a rat, a dog, a naked mole rat, a nonhuman primate, and a human. In some embodiments, a mammal can be a mouse. In some embodiments, a mammal can be a rat. In some embodiments, a mammal can be a rabbit. In some embodiments, a mammal can be a cat. In some embodiments, a mammal can be a nonhuman primate (e.g., a chimpanzee, a gorilla, an orangutan, a rhesus monkey, a cynomolgus monkey, a Taiwanese macaque, a green monkey, a squirrel monkey, tamarin, a marmoset, or a mouse lemur). In some embodiments, a mammal can be a pig. In some embodiments, a mammal can be a human. In some embodiments, an animal can be an animal model of a heart disorder (e.g., any of the heart disorders described herein). In some embodiments, a mammal can be a mammalian model of a heart disorder (e.g, any of the heart disorders described herein or known in the art).

In some embodiments, an animal model for a heart disorder is a ligation-induced myocardial infarction (MI) mouse model. In some embodiments, an animal model for a heart disorder is a cryogenic injury mouse model that induceds confluent necrosis for studying heart regeneration and cellular remodeling. In some embodiments, an animal model for a heart disorder or disorder is a doxorubicin (DOX)-induced heart failure (HF) model.

In some embodiments, an animal model of a heart disorder can express one or more human genes. Animal models of heart disorders can include one or more mutations in one or more genes. In some embodiments, an animal model for a heart disorder is a transgenic animal model. In some embodiments, an animal model for a heart disorder is a dialated cardiomyopathy (DCM) mouse model with an MLP^{-/-} genotype. In some embodiments, an animal model for a heart disorder is a hypertrophic cardiomyopathy (HCM) mouse model in which cMyBP-C is homozygously ablated (cMyBP-C^{-/-}). In some embodiments, an animal model for a heart disorder is an autoimmune cardiomyopathy (AICM) and heart failure (HF) with a DQ8^{+/+}, IAβ^{-/-} NOD genotype. In some embodiments, an animal model for a heart disorder is a Duchenne's muscle dystrophy (DMD) model with a mutation in the dystrophin gene. In some embodiments, an animal model for a heart disorder is an atrial fibrillation (AF) mouse model with a cardiac-specific LKB1 knockout. In some embodiments, an animal model for a heart disorder is a ligation-induced myocardial infarction (MI) rat model. In some embodiments, an animal model for a heart disorder is an overload-induced cardiac hypertrophy rat model. In some embodiments, an animal model for a heart disorder is a diabetic cardiomyopathy (DbCM) rat model. In some embodiments, an animal model for a heart disorder is a transgenic rat model of myocardial infarction in hypertensive rats. In some embodiemnts, an animal model for a heart disorder is a Type II diabetes Goto-Kakizaki (GK) rat. In some embodiments, an animal model for a heart disorder is a JCR:LA-cp rat. In some embodiments, an animal model for a heart disorder is a Duchenne's muscular dystrophy (DMD) rat generated by microinjecting a mixture of TALE nuclease mRNA for DMD in rat zygotes. In some embodiments, an animal model for a heart disorder is a spontaneous Watanabe heritable hyperlipidemic myocardial infarction (WHHL-MI) rabbit model. In some embodiments, an animal model for a heart disorder is a hypertrophic cardiomyopathy (HCM) cat model. Other animal models of heart disorders are known in the art (see, for example, Camacho, et al., Am. J. Cardiovasc. Dis. 6(3):70-80, 2016).

(c) Biomarkers and Candidate Biomarkers

As used herein, a biomarker can be any appropriate biomarker. In some embodiments, a biomarker can be a nucleic acid (e.g., genomic DNA (gDNA), mRNA, or rRNA (e.g., bacterial 16S rRNA)), a protein, a peptide, or a fragment thereof, (e.g., an enzyme, a cell surface marker, a structural protein, a tumor suppressor, an antibody, a cytokine, a peptide hormone, or an identifiable fragment, precursor, or degredation product of any thereof), a lipoprotein, a cell (e.g., a cell type, for example, in a location indicative of disease), or a small molecule (e.g., an enzymatic cofactor, a hormone (e.g., a steroid hormone or a eicosanoid hormone), or a metabolite). In some embodiments, a biomarker can include an alteration in a nucleic acid (e.g., an insertion, a deletion, a point mutation, and/or methylation), for example, relative to a wildtype or control nucleic acid. In some embodiments, a biomarker can include an alteration in a protein (e.g., an inserted amino acid, a deletion of an amino acid, an amino acid substitution, and/or a post-translational modification (e.g., presence, absence, or a change in, for example, acylation, isoprenylation, phosphorylation, glycosylation, methylation, hydroxylation, amidation, and/or ubiquitinylation)), for example, relative to a control or wildtype protein.

In some embodiments, a biomarker is a nucleic acid. In some embodiments, a biomarker is an mRNA. In some embodiments, a biomarker is a protein. In some embodiments, a biomarker is an enzyme. In some embodiments, a biomarker is a cell surface marker. In some embodiments, a biomarker can be one or more analytes selected from (1) ribosome protein L17 (RPL17), (2) ribosomal protein L36a (RPL36A), (3) thymosin beta 4 X-linked (TMSB4X), (4) coiled-coil domain containing 80 (CCDC80), (5) ferritin light chain (FTL), (6) ribosomal protein L37 (RPL37), (7) heme oxygenase 1 (HMOX1), (8) Y-box binding protein 1 (YBX1), (9) ribosomal protein S17 (RPS17), (10) ribosomal protein S29 (RPS29), (11) pancreatic progenitor cell differentiation and proliferation factor (PPDPF), (12) Y-box binding protein 3 (YBX3), (13) vimentin (VIM), (14) NADH:ubiquinone oxidoreductase subunit B1 (NDUFB1), (15) ribosomal protein L34 (RPL34), (16) macrophage migration inhibitory factor (MIF), (17) phospholipid transfer protein (PLTP), (18) ribosomal protein L38 (RPL38), (19) ribosomal protein S21 (RPS21), (20) ribosomal protein S10 (RPS10), (21) ribonuclease A family member 1, pancreatic (RNASE1), (22) ribosomal protein S25 (RPS25), (23) ribosomal protein L26 (RPL26), (24) ribosomal protein L37a (RPL37A), (25) ribosomal protein S15a (RPS15A), (26) eukaryotic translation elongation factor 1 alpha 2 (EEF1A2), (27) ribosomal protein S27 (RPS27), (28) nexilin F-actin binding protein (NEXN), (29) collagen type I alpha 1 chain (COL1A1), (30) ribosomal protein L23 (RPL23), (31) collagen type III alpha 1 chain (COL3A1), (32) ATP synthase F1 subunit epsilon (ATP5F1E), (33) ribosomal protein S8 (RPS8), (34) ribosomal protein L31 (RPL31), (35) poly(A) binding protein cytoplasmic 1 (PABPC1), (36) ribosomal protein S28 (RPS28), (37) fatty acid binding protein 4 (FABP4), (38) decorin (DCN), (39) matrix Gla protein (MGP), (40) ribosomal protein L22 (RPL22), (41) ribosomal protein L39 (RPL39), (42) complement C1q A chain (C1QA), (43) complement C1q B chain (C1QB), (44) myosin heavy chain 6 (MYH6), (45) secreted protein acidic and cysteine rich (SPARC), (46) translation machinery associated 7 homolog (TMA7), (47) ribosomal protein L23a (RPL23A), (48) NADH:ubiquinone oxidoreductase subunit A1 (NDUFA1), (49) cytochrome c oxidase subunit 7C (COX7C), (50) ferritin heavy chain 1 (FTH1), and byproducts, degradation products, or precursors thereof.

(d) Locations in a Sample

As used herein, a location in a sample can be any appropriate location. For example, in some embodiments, a heart disorder is a condition, disorder, or disease that occurs in a location within a heart tissue. In some embodiments, a heart tissue can be selected from the group of brachiocephalic trunk, left common carotid artery, left subclavian artery, aortic arch, aorta, superior vena cava, right pulmonary artery, left pulmonary artery, ligamentum arteriosum, right pulmonary veins, left pulmonary veins, ascending aorta, pulmonary trunk, auricle of left atrium, right atrium, left atrium, circumflex artery, inferior vena cava, right coronary artery, left coronary artery, anterior cardiac vein, coronary sinus, circumflex branch of left coronary artery, right ventricle, left ventricle, small cardiac vein, great cardiac vein, middle cardiac vein, right marginal artery, anterior interventricular artery, or posterior interventricular artery. In some embodiments, a heart disorder can be detected or diagnosed in a heart tissue section. In some embodiments, a heart disorder can be detected or diagnosed in a tissue or biological sample other than a heart tissue section, for example, a blood sample, a plasma sample, or a serum sample.

(e) Reference Levels

A reference level of a biomarker can be any appropriate reference level. In some embodiments, a reference level of a biomarker can be determined based on a level of the biomarker in a corresponding sample (e.g., a heart of a control animal, e.g., a control animal not diagnosed, not presenting with any of the symptoms of a heart disorder, not having a family history of a heart disorder, and not having any known risk factors of a heart disorder) at a corresponding position. In some embodiments, a reference level of a biomarker can be determined based on an amount of the biomarker in one or more other locations in a sample. In some embodiments, a reference level of the biomarker can be determined based on a level of the biomarker in a different location within the same tissue sample (e.g., the same heart section). In some embodiments, a reference level of a biomarker can be determined based on a level of the biomarker in a healthy portion within the same tissue sample as the diseased portion (e.g., a healthy portion within the same heart section as the diseased portion).

In some embodiments, a reference level can be based on a reference level as published by an appropriate body (e.g., a government agency (e.g., the United States Food and Drug Administration) or a professional organization (e.g., the American Medical Association or American Heart Association)), for example, a reference level that is a threshold level for a biomarker at the location in the heart of an animal.

In some embodiments, a reference level of a biomarker can be determined based on any appropriate criteria. For example, in some embodiments, a reference level of a biomarker can come from an age-matched healthy subject. In some embodiments, a reference level of a biomarker can come from a sex-matched healthy subject or a sex-matched healthy subject population. In some embodiments, a reference level of a biomarker can come from an age-matched, sex-matched healthy subject or an age-matched, sex-matched healthy subject population. In some embodiments, a reference level of a biomarker can come from an aggregate sample (e.g., an average of 2 or more individual) of healthy subjects (e.g., that are age-matched and/or sex-matched).

A healthy subject can be any appropriate healthy subject. In some embodiments, a healthy subject has one or more of: no known heart disorder, presentation of no symptoms or no more than three (e.g., no more than two, or no more than one) of: a heart disorder, no known genetic mutations associated with risk of a heart disorder, no family medical history of a heart disorder, and no behavioral risk factors of a heart disorder.

In some cases, a level of a biomarker can be elevated relative to a reference level. For example, a level of a biomarker can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference level (e.g., any of the exemplary reference levels described herein or known in the art).

In some cases, a level of a biomarker can be decreased relative to a reference level. For example, a level of a biomarker can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, about a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference level (e.g., any of the exemplary reference levels described herein). Other suitable reference levels and methods of determining the same will be apparent to those skilled in the field.

(f) Identifying a Diagnostic or Prognostic Marker of Myocardial Infarction or a Candidate Biomarker for Efficacy of a Treatment of a Brain Disorder

In some embodiments, provided herein are methods for identifying a diagnostic or prognostic biomarker of a heart disorder (e.g., a myocardial infarction). Also provided herein are methods for determining a candidate biomarker for determining efficacy of a treatment of a heart disorder. A diagnostic or prognostic biomarker is an analyte (e.g., nucleic acid, protein) that can be used to identify and/or determine the presence of a heart disorder, or determine the likelihood that a heart disorder can or will be identified in a subject. In some instances, candidate prognostic biomarker is detected and used to predict the prognosis of a subject's heart disorder. In some instances, the diagnostic or prognostic biomarker is increased relative to a reference sample. In some instances, the diagnostic or prognostic biomarker is decreased relative to a reference sample.

The methods can include (a) determining level(s) of one or more biomarker(s) in a location in a sample comprising heart tissue obtained from an animal having a heart disorder; (b) identifying: (i) one or more biomarker(s) showing elevated level(s) in the location in the sample as compared to reference level(s) of the one or more biomarker(s) and/or (ii) one or more biomarker(s) showing decreased level(s) in the location in the sample as compared to reference level(s) of the one or more biomarker(s) as diagnostic, or prognostic biomarker(s) of the heart disorder and/or or as candidate biomarker(s) for determining efficacy of a treatment of the heart disorder. In some embodiments, a reference level of the one or more biomarker(s) is a level of the one or more biomarker(s) in a corresponding location in a sample comprising heart tissue obtained from a control animal. In some embodiments, an animal can be any of the animals described herein. In some embodiments, an animal can be a mammal.

In some embodiments, the methods can include identifying one or more biomarker(s) showing elevated level(s) in the location in the sample as compared to reference level(s) of the one or more biomarker(s) as diagnostic, or prognostic biomarker(s) of the heart disorder. In some embodiments, the methods can include identifying one or more biomarker(s) showing decreased level(s) in the location in the sample as compared to reference level(s) of the one or more biomarker(s) (as diagnostic or prognostic biomarker(s) of the heart disorder.

The identified diagnostic markers (identified using the methods described herein) can then be used in methods of diagnosing a heart disorder (e.g., by measuring a level of the identified diagnostic marker in a biological sample obtained from a subject (e.g., a biological sample comprising blood, serum, plasma or tissue), and comparing the level to a reference level (e.g., any of the exemplary reference levels described in herein (e.g., a level of the biomarker in a similar biological sample from a control subject or a population of control subjects)). For example, an increase in the level of the diagnostic biomarker (e.g., one or more analytes selected from RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, or a byproduct or precursor or degradation product thereof) in a biological sample obtained from a subject (for a diagnostic biomarker identified as having an increased level as compared to a reference level) as compared to a level of the biomarker in a similar biological sample from a control subject or a population of control subjects indicates that the subject has heart disorder. For example, a decrease in the level of the diagnostic biomarker (e.g., one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof) in a biological sample obtained from a subject (for a diagnostic biomarker identified as having an decreased level as compared to a reference level) as compared to a level of the biomarker in a similar biological sample from a control subject or a population of control subjects indicates that the subject has the heart disorder. Such methods can be used to identify patients having an early stage of a heart disorder (e.g., before the presentation of symptoms). In such methods where a patient is diagnosed as having an early stage of a heart disorder (e.g., using any of the methods described herein), the subject can be administered a treatment that allows for a decrease in the rate of progression of the heart disorder in the subject.

In some instances, the methods of identifying a marker (or biomarker) include the following steps. In some instances, a biological sample is placed on a substrate that comprises a plurality of capture probes. After permeabilization of the biological sample (e.g., using a solution comprising proteinase K), analytes (e.g., mRNA molecules) migrate and hybridize to the capture probe. In some instances, the capture probe includes a capture domain that includes a poly-thymine (T) sequence that can indiscriminately hybridize to a poly(A) mRNA sequence of an analyte. Once the capture probes capture the analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps. The library preparation can optionally be quality controlled to verify the success of the library preparation methods. The cDNA fragments can then be sequenced using, for example, paired-end sequencing using TruSeq Read 1 and TruSeq Read 2 as sequencing primer sites.

In some aspects, arrays (e.g., glass slides) include a plurality of capture probes that bind (via hybridization or ligation) to one or more specific biological targets in a sample (i.e., targeted analysis). In some aspects, the capture probes hybridize to specific analytes, e.g., under appropriate conditions where oligonucleotide capture probes can hybridize to the target nucleic acids in a sequence-specific manner. That is, the capture probe includes a sequence that is specific to an analyte of interest, and the capture probe discriminately captures the targeted analyte. In some instances, the capture probe comprises a sequence that is substantially complementary to a targeted analyte. In some instances, a splint oligonucleotide is used. In some instances, the splint oligonucleotide hybridizes to the targeted analyte and the capture probe, bringing the molecules in close proximity. Then, in some instances, the targeted probe and capture probe are joined (e.g., by ligation or by extension of one of the targeted analyte or the capture probe followed by ligation). In some aspects, analytes that are not captured by the capture probes are removed (e.g., analytes that do not interact with capture domains of the capture probes). In some embodiments, removal of analytes that did not interact with a capture probe can be accomplished by, e.g., washing the sample to remove such analytes.

In some instances, targeted capture occurs through enrichment of targets of interest after analytes are non-discriminately captured by capture probes on an array. In this instance, analytes (e.g., mRNA) is captured by a capture probe. In some instances, the capture probe includes a sequence that hybridizes to an analyte. In some instances, the capture probe includes poly-thymine (T) sequence that hybridizes to a poly(A) sequence of an mRNA analyte. After the analytes are captured by the capture probe, the analytes are pooled and amplified. In some instances, after amplification, specific analytes of interest are enriched in the pool. In some instances, a plurality of bait oligonucleotides are added to the pool. In some instance, a bait oligonucleotide includes a capture domain that binds specifically to all or a portion of the sequence of the nucleic acid from the biological sample, or a complement thereof. In some instances, the bait oligonucleotide includes a molecular tag. In some instances, the molecular tag include a moiety such as a streptavidin molecule, an avidin molecule, a biotin molecule, or a fluorophore molecule. In some instances, the moiety can be used to isolate bait oligonucleotides that have hybridized to a target sequence of interest. After isolation of the hybridized bait oligonucleotide/target, the target can be isolated, purified, and optionally amplified using methods known in the art. In some instances, this enriched pool of a target of interest can then be sequenced to identify all or a portion of the sequence of the spatial barcode (from the initial capture probe) or the complement thereof, and all or a portion of the sequence of the nucleic acid from the biological sample, and using the determined sequences of (i) and (ii) to identify the location of the nucleic acid in the biological sample.

In some instances, a plurality of bait oligonucleotides can be designed so that each bait oligonucleotide sequence theoretically hybridizes to a unique target of interest. In some instances, the designed bait oligonucleotides are at least 40 nucleotides in length. In some instances, the bait oligonucleotides are about 120 nucleotides in length. In some instances, the bait oligonucleotides range from about 40 to about 160 nucleotides in length. In some instances, a panel of bait oligonucleotides are used to target one analyte of interest or a plurality of analytes of interest. In some embodiments, the plurality of analytes of interest is between five genes and twenty thousand genes. In some embodiments, the plurality of analytes is between one hundred genes and ten thousand genes. In some embodiments, the plurality of analytes is between five hundred analytes and two thousand analytes. In some embodiments, the plurality of analytes is more than 10, more than 50, more than 100, more than 500, more than 1000, more than 2000, more than 5000, more than 10000, more than 15000, or more than 20000 analytes. It is appreciated that panels and bait oligonucleotides can be designed to target analytes of interest in a specific setting (e.g., for a specific tissue or for a specific pathological setting such as cancer),In some cases, spatial analysis can be performed by detecting multiple oligonucleotides that hybridize to one or more analytes. In some instances, for example, spatial analysis can be performed using RNA-templated ligation (RTL). Methods of in situ hybridization such as RTL have been described previously. See e.g., Credle et al., Nucleic Acids Res. 2017 Aug. 21; 45(14):e128. Briefly, RTL steps include hybridization of two oligonucleotides to adjacent sequences of an analyte (e.g., an RNA molecules, e.g., an mRNA molecule). In some instances, the oligonucleotides are DNA molecules. In some instances, one of the oligonucleotides includes at least two ribonucleic acid bases at the 3′ end and the other oligonucleotide includes a phosphorylated nucleotide at the 5′ end. In some instances, one of the two oligonucleotides includes a capture probe binding domain (e.g., a poly(A) sequence).

After hybridization, a ligase (e.g., T4 DNA ligase) ligates the oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. In some instances, after ligation, the ligation product is released from the analyte. In some instances, the ligation product is released using an endonuclease (e.g., an RNAse, e.g., RNase A, RNase C, RNase H, or RNase I). The released ligation product can then be captured by capture probes on an array, amplified, and sequenced, thus determining the location and abundance of the analyte in the biological sample.

In some embodiments, the methods include optimizing permeabilization of a biological sample. Optimizing permeabilization can be useful for identifying intracellular analytes. Permeabilization optimization can include selection of permeabilization agents, concentration of permeabilization agents, and permeabilization duration. In general, a biological sample can be permeabilized by exposing the sample to one or more permeabilizing agents. Suitable agents for this purpose include, but are not limited to, organic solvents (e.g., acetone, ethanol, and methanol), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized during any of the steps described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)).

At any point during the methods disclosed herein, the biological sample can be imaged. For example, a region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof.

In some embodiments, this disclosure further provides devices for holding or supporting substrates for use in the methods disclosed herein. In particular, the devices include a first and second members that receive a first and second substrate, respectively. In some embodiments, the devices of the disclosure can be used for sandwiching the first and second substrates together for spatial analysis applications. In some embodiments, the first substrate can support a sample (e.g., a biological substrate) on its surface. In some embodiments, the second substrate can include a plurality of barcoded probes and/or permeabilization reagents. In some instances, the biological sample is permeabilized to allow analytes to be released from the sample on the first substrate and bind (e.g., hybridize) to the capture probes attached to the second substrate.

In some instances, the sandwiching processes between a first substrate comprising a biological sample (e.g., a tissue section on a slide) and a second substrate comprising a spatially barcoded array, e.g., a slide that is populated with spatially-barcoded capture probes. During the exemplary sandwiching process, the first substrate is aligned with the second substrate, such that at least a portion of the biological sample is aligned with at least a portion of the array (e.g., aligned in a sandwich configuration). As shown, the slide is in a superior position to the pathology slide. In some embodiments, the pathology slide may be positioned superior to the slide. In some embodiments, the first and second substrates are aligned to maintain a gap or separation distance between the two substrates. When the first and second substrates are aligned, one or more analytes are released from the biological sample and actively or passively migrate to the array for capture. In some embodiments, the migration occurs while the aligned portions of the biological sample and the array are contacted with a reagent medium. The released one or more analytes may actively or passively migrate across the gap via the reagent medium toward the capture probes, and be captured by the capture probes. Additional methods and devices relating to substrates that are used in sandwiching methods are disclosed in WO 2020/123320, which in incorporated by reference in its entirety.

In some instances, also disclosed is a method for quantitatively profiling gene expression signatures correlating to a disease state of a subject, wherein the disease state is a heart disease, comprising: generating a profile of expression levels of a plurality of analytes, wherein an analyte in the plurality of analytes is correlated with the heart disease in a biological sample obtained from the subject, wherein the profile is generated from a library generated by: (a) contacting the biological sample with an substrate comprising a plurality of attached capture probes, wherein a capture probe of the plurality of attached capture probes comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe. In some embodiments, the methods can include performing an experiment to validate whether the one or more identified candidate prognostic biomarker(s) provides for an accurate assessment of the prognosis of the heart disorder in a mammal. Non-limiting examples of experiments can include generation of a knock-out (e.g., a knock-out of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, and RPL38) or a knock-in (e.g., a knock-in of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, and FTH1) animal, and study of the prognosis of the knockout or knock-in animal. In some embodiments, the additional experiments can include following a group of patients having a heart disorder over time and assessing the level of the biomarker in the subject over time. In some embodiments, the additional experiments can include determining the level of the biomarker in an animal model of a heart disorder over time. Other experiments to validate whether the one or more identified candidate prognostic biomarker(s) will be apparent to those skilled in the field.

In some embodiments, the methods can further include performing an experiment to validate whether the one or more identified candidate biomarker(s) provides for an accurate assessment of the efficacy of a treatment of the heart disorder in an animal. Non-limiting examples of experiments can include administering a treatment of a heart disorder to an animal model of the heart disorder and assessing the levels of the identified candidate biomarker in the animal model over time and assessing progression of the disease in the animal model over time). Other experiments to validate whether the one or more identified candidate biomarker(s) provides for an accurate assessment of the efficacy of a treatment of the heart disorder in an animal will be apparent to those skilled in the field.

(g) Biomarkers of Myocardial Infarction

As described in Example 1, ribosome protein L17 (RPL17), ribosomal protein L36a (RPL36A), thymosin beta 4 X-linked (TMSB4X), coiled-coil domain containing 80 (CCDC80), ferritin light chain (FTL), ribosomal protein L37 (RPL37), heme oxygenase 1 (HMOX1), Y-box binding protein 1 (YBX1), ribosomal protein S17 (RPS17), ribosomal protein S29 (RPS29), pancreatic progenitor cell differentiation and proliferation factor (PPDPF), Y-box binding protein 3 (YBX3), vimentin (VIM), NADH:ubiquinone oxidoreductase subunit B1 (NDUFB1), ribosomal protein L34 (RPL34), macrophage migration inhibitory factor (MIF), phospholipid transfer protein (PLTP), ribosomal protein L38 (RPL38), ribosomal protein S21 (RPS21), ribosomal protein S10 (RPS10), ribonuclease A family member 1, pancreatic (RNASE1), ribosomal protein S25 (RPS25), ribosomal protein L26 (RPL26), ribosomal protein L37a (RPL37A), ribosomal protein S15a (RPS15A), eukaryotic translation elongation factor 1 alpha 2 (EEF1A2), ribosomal protein S27 (RPS27), nexilin F-actin binding protein (NEXN), collagen type I alpha 1 chain (COL1A1), ribosomal protein L23 (RPL23), collagen type III alpha 1 chain (COL3A1), ATP synthase F1 subunit epsilon (ATP5F1E), ribosomal protein S8 (RPS8), ribosomal protein L31 (RPL31), poly(A) binding protein cytoplasmic 1 (PABPC1), ribosomal protein S28 (RPS28), fatty acid binding protein 4 (FABP4), decorin (DCN), matrix Gla protein (MGP), ribosomal protein L22 (RPL22), ribosomal protein L39 (RPL39), complement C1q A chain (C1QA), complement C1q B chain (C1QB), myosin heavy chain 6 (MYH6), secreted protein acidic and cysteine rich (SPARC), translation machinery associated 7 homolog (TMA7), ribosomal protein L23a (RPL23A), NADH:ubiquinone oxidoreductase subunit A1 (NDUFA1), cytochrome c oxidase subunit 7C (COX7C), and ferritin heavy chain 1 (FTH1) have been identified as diagnostic biomarkers of myocardial infarction.

Some embodiments of any of the methods described herein can include the detection of a level of one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof.

In some embodiments of any of the methods described herein, a biomarker can be one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof.

In some embodiments of any of the methods described herein, a biomarker can be genomic DNA, mRNA, or protein. In some embodiments of any of the methods described herein, a biomarker can be a nucleic acid including a sequence that is at least 80% identical (e.g., at least 85%, 90%, 95%, 98%, 99%, or is 100% identical) to any one of SEQ ID NOs. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, and 101. In some embodiments of any of the methods described herein, a biomarker can be a nucleic acid including a sequence that encodes a polypeptide that is at least 80% identical (e.g., at least 85%, 90%, 95%, 98%, 99%, or is 100% identical) to any one of SEQ ID NOs. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 801 82, 84, 86, 88, 90, 92, 94, 96, 98, 100 and 102. In some embodiments of any of the methods described herein, a biomarker can be a polypeptide that is at least 80% identical (e.g., at least 85%, 90%, 95%, 98% or 99% identical) to any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 801 82, 84, 86, 88, 90, 92, 94, 96, 98, 100, and 102.

TABLE 1

| Biomarker | SEQ ID NO | Description |
|---|---|---|
| RPL17 | SEQ ID NO: 1 | >NM_001199342.3 Homo sapiens ribosomal protein L17 |
| | | (RPL17), transcript variant 5, mRNA |
| RPL17 | SEQ ID NO: 2 | >NP_001186271.1 60S ribosomal protein L17 isoform a [Homo |
| | | sapiens] |
| RPL36A | SEQ ID NO: 3 | >NM_021029.6 Homo sapiens ribosomal protein L36a (RPL36A), |
| | | mRNA |
| RPL36A | SEQ ID NO: 4 | >NP_066357.3_xxx ribosomal protein L36a (RPL36A), [Homo |
| | | sapiens] |
| TMSB4X | SEQ ID NO: 5 | >NM_021109.4 Homo sapiens thymosin beta 4 X-linked (TMSB4X), |
| | | mRNA |
| TMSB4X | SEQ ID NO: 6 | >NP_066932.1 thymosin beta 4 X-linked (TMSB4X) [Homo sapiens] |
| CCDC80 | SEQ ID NO: 7 | >NM_199511.2 Homo sapiens coiled-coil domain containing 80 |
| | | (CCDC80), transcript variant 1, mRNA |
| CCDC80 | SEQ ID NO: 8 | >NP_955805.1 coiled-coil domain-containing protein 80 precursor |
| | | [Homo sapiens] |
| FTL | SEQ ID NO: 9 | >NM_000146.4 Homo sapiens ferritin light chain (FTL), mRNA |
| FTL | SEQ ID NO: 10 | >NP_000137.2 ferritin light chain [Homo sapiens] |
| RPL37 | SEQ ID NO: 11 | >NM_000997.5 Homo sapiens ribosomal protein L37 (RPL37), |
| | | transcript variant 1, mRNA |
| RPL37 | SEQ ID NO: 12 | >NP_000988.1 60S ribosomal protein L37 [Homo sapiens] |
| HMOX1 | SEQ ID NO: 13 | >NM_002133.3 Homo sapiens heme oxygenase 1 (HMOX1), mRNA |
| HMOX1 | SEQ ID NO: 14 | >NP_002124.1 heme oxygenase 1 [Homo sapiens] |
| YBX1 | SEQ ID NO: 15 | >BC106045.1 Homo sapiens Y box binding protein 1, mRNA (cDNA |
| | | clone MGC: 117250 IMAGE: 6062615), complete cds |
| YBX1 | SEQ ID NO: 16 | >NP_004550.2 Y-box-binding protein 1 [Homo sapiens] |
| RPS17 | SEQ ID NO: 17 | >NM_001021.6 Homo sapiens ribosomal protein S17 (RPS17), |
| | | transcript variant 1, mRNA |
| RPS17 | SEQ ID NO: 18 | >NP_001012.1 40S ribosomal protein S17 [Homo sapiens] |
| RPS29 | SEQ ID NO: 19 | >NM_001032.4 Homo sapiens ribosomal protein S29 (RPS29), |
| | | transcript variant 1, mRNA |
| RPS29 | SEQ ID NO: 20 | >NP_001023.1 40S ribosomal protein S29 isoform 1 [Homo sapiens] |
| PPDPF | SEQ ID NO: 21 | >NM_024299.4 Homo sapiens pancreatic progenitor cell |
| | | differentiation and proliferation factor (PPDPF), transcript variant 1, |
| | | mRNA |
| PPDPF | SEQ ID NO: 22 | >NP_077275.1 pancreatic progenitor cell differentiation and |
| | | proliferation factor isoform 1 [Homo sapiens] |
| YBX3 | SEQ ID NO: 23 | >NM_003651.5 Homo sapiens Y-box binding protein 3 (YBX3), |
| | | transcript variant 1, mRNA |
| YBX3 | SEQ ID NO: 24 | >NP_003642.3 Y-box-binding protein 3 isoform a [Homo sapiens] |
| VIM | SEQ ID NO: 25 | >NM_003380.5 Homo sapiens vimentin (VIM), mRNA |
| VIM | SEQ ID NO: 26 | >NP_003371.2 vimentin [Homo sapiens] |
| NDUFB1 | SEQ ID NO: 27 | >NM_004545.4 Homo sapiens NADH: ubiquinone oxidoreductase |
| | | subunit B1 (NDUFB1), mRNA |
| NDUFB1 | SEQ ID NO: 28 | >NP_004536.3 NADH dehydrogenase [ubiquinone] 1 beta |
| | | subcomplex subunit 1 [Homo sapiens] |
| RPL34 | SEQ ID NO: 29 | >NM_001319235.1 Homo sapiens ribosomal protein L34 (RPL34), |
| | | transcript variant 5, mRNA |
| RPL34 | SEQ ID NO: 30 | >NP_001306164.1 60S ribosomal protein L34 [Homo sapiens] |
| MIF | SEQ ID NO: 31 | >NM_002415.2 Homo sapiens macrophage migration inhibitory |
| | | factor (MIF), mRNA |
| MIF | SEQ ID NO: 32 | >NP_002406.1 macrophage migration inhibitory factor [Homo |
| | | sapiens] |
| PLTP | SEQ ID NO: 33 | >NM_006227.4 Homo sapiens phospholipid transfer protein (PLTP), |
| | | transcript variant 1, mRNA |
| PLTP | SEQ ID NO: 34 | >NP_006218.1 phospholipid transfer protein isoform a precursor |
| | | [Homo sapiens] |
| RPL38 | SEQ ID NO: 35 | >NM_000999.4 Homo sapiens ribosomal protein L38 (RPL38), |
| | | transcript variant 1, mRNA |
| RPL38 | SEQ ID NO: 36 | >NP_000990.1 60S ribosomal protein L38 [Homo sapiens] |
| RPS21 | SEQ ID NO: 37 | >NM_001024.4 Homo sapiens ribosomal protein S21 (RPS21), |
| | | mRNA |
| RPS21 | SEQ ID NO: 38 | >NP_001015.1 40S ribosomal protein S21 [Homo sapiens] |
| RPS10 | SEQ ID NO: 39 | >NM_001014.5 Homo sapiens ribosomal protein S10 (RPS10), |
| | | transcript variant 2, mRNA |
| RPS10 | SEQ ID NO: 40 | >NP_001005.1 40S ribosomal protein S10 [Homo sapiens] |
| RNASE1 | SEQ ID NO: 41 | >NM_002933.5 Homo sapiens ribonuclease A family member 1, |
| | | pancreatic (RNASE1), transcript variant 4, mRNA |
| RNASE1 | SEQ ID NO: 42 | >AAH05324.1 Ribonuclease, RNase A family, 1 (pancreatic) |
| | | [Homo sapiens] |
| RPS25 | SEQ ID NO: 43 | >NM_001028.3 Homo sapiens ribosomal protein S25 (RPS25), |
| | | mRNA |
| RPS25 | SEQ ID NO: 44 | >NP_001019.1 40S ribosomal protein S25 [Homo sapiens] |
| RPL26 | SEQ ID NO: 45 | >NM_000987.5 Homo sapiens ribosomal protein L26 (RPL26), |
| | | transcript variant 2, mRNA |
| RPL26 | SEQ ID NO: 46 | >NP_000978.1 60S ribosomal protein L26 [Homo sapiens] |
| RPL37A | SEQ ID NO: 47 | >NM_000998.5 Homo sapiens ribosomal protein L37a (RPL37A), |
| | | mRNA |
| RPL37A | SEQ ID NO: 48 | >NP_000989.1 60S ribosomal protein L37a [Homo sapiens] |
| RPS15A | SEQ ID NO: 49 | >NM_001030009.2 Homo sapiens ribosomal protein S15a |
| | | (RPS15A), transcript variant 1, mRNA |
| RPS15A | SEQ ID NO: 50 | >NP_001010.2 40S ribosomal protein S15a [Homo sapiens] |
| EEF1A2 | SEQ ID NO: 51 | >NM_001958.5 Homo sapiens eukaryotic translation elongation |
| | | factor 1 alpha 2 (EEF1A2), mRNA |
| EEF1A2 | SEQ ID NO: 52 | >NP_001949.1 elongation factor 1-alpha 2 [Homo sapiens] |
| RPS27 | SEQ ID NO: 53 | >NM_001030.6 Homo sapiens ribosomal protein S27 (RPS27), |
| | | transcript variant 1, mRNA |
| RPS27 | SEQ ID NO: 54 | >NP_001021.1 40S ribosomal protein S27 isoform 1 [Homo sapiens] |
| NEXN | SEQ ID NO: 55 | >NM_144573.3 Homo sapiens nexilin F-actin binding protein |
| | | (NEXN), transcript variant 1, mRNA |
| NEXN | SEQ ID NO: 56 | >NP_653174.3 nexilin isoform 1 [Homo sapiens] |
| COL1A1 | SEQ ID NO: 57 | >NM_000088.4 Homo sapiens collagen type I alpha 1 chain |
| | | (COL1A1), mRNA |
| COL1A1 | SEQ ID NO: 58 | >NP_000079.2 collagen alpha-1(I) chain preproprotein [Homo |
| | | sapiens] |
| RPL23 | SEQ ID NO: 59 | >NM_000978.4 Homo sapiens ribosomal protein L23 (RPL23), |
| | | mRNA |
| RPL23 | SEQ ID NO: 60 | >NP_000969.1 60S ribosomal protein L23 [Homo sapiens] |
| COL3A1 | SEQ ID NO: 61 | >NM_000090.3 Homo sapiens collagen type III alpha 1 chain |
| | | (COL3A1), mRNA |
| COL3A1 | SEQ ID NO: 62 | >AGL34959.1 collagen type III alpha 1 [Homo sapiens] |
| ATP5F1E | SEQ ID NO: 63 | >NM_006886.4 Homo sapiens ATP synthase F1 subunit epsilon |
| | | (ATP5F1E), mRNA |
| ATP5F1E | SEQ ID NO: 64 | >NP_008817.1 ATP synthase subunit epsilon, mitochondrial [Homo |
| | | sapiens] |
| RPS8 | SEQ ID NO: 65 | >NM_001012.2 Homo sapiens ribosomal protein S8 (RPS8), mRNA |
| RPS8 | SEQ ID NO: 66 | >NP_001003.1 40S ribosomal protein S8 [Homo sapiens] |
| RPL31 | SEQ ID NO: 67 | >NM_000993.5 Homo sapiens ribosomal protein L31 (RPL31), |
| | | transcript variant 1, mRNA |
| RPL31 | SEQ ID NO: 68 | >NP_000984.1 60S ribosomal protein L31 isoform 1 [Homo sapiens] |
| PABPC1 | SEQ ID NO: 69 | >NM_002568.4 Homo sapiens poly(A) binding protein cytoplasmic 1 |
| | | (PABPC1), mRNA |
| PABPC1 | SEQ ID NO: 70 | >NP_002559.2 polyadenylate-binding protein 1 [Homo sapiens] |
| RPS28 | SEQ ID NO: 71 | >NM_001031.5 Homo sapiens ribosomal protein S28 (RPS28), |
| | | mRNA |
| RPS28 | SEQ ID NO: 72 | >NP_001022.1 40S ribosomal protein S28 [Homo sapiens] |
| FABP4 | SEQ ID NO: 73 | >NM_001442.3 Homo sapiens fatty acid binding protein 4 (FABP4), |
| | | mRNA |
| FABP4 | SEQ ID NO: 74 | >NP_001433.1 fatty acid-binding protein, adipocyte [Homo sapiens] |
| DCN | SEQ ID NO: 75 | >NM_001920.5 Homo sapiens decorin (DCN), transcript variant A1, |
| | | mRNA |
| DCN | SEQ ID NO: 76 | >NP_001911.1 decorin isoform a preproprotein [Homo sapiens] |
| MGP | SEQ ID NO: 77 | >NM_000900.5 Homo sapiens matrix Gla protein (MGP), transcript |
| | | variant 2, mRNA |
| MGP | SEQ ID NO: 78 | >NP_000891.2 matrix Gla protein isoform 2 preproprotein [Homo |
| | | sapiens] |
| RPL22 | SEQ ID NO: 79 | >NM_000983.4 Homo sapiens ribosomal protein L22 (RPL22), |
| | | mRNA |
| RPL22 | SEQ ID NO: 80 | >NP_000974.1 60S ribosomal protein L22 [Homo sapiens] |
| RPL39 | SEQ ID NO: 81 | >NM_001000.4 Homo sapiens ribosomal protein L39 (RPL39), |
| | | mRNA |
| RPL39 | SEQ ID NO: 82 | >NP_000991.1 60S ribosomal protein L39 [Homo sapiens] |
| C1QA | SEQ ID NO: 83 | >NM_015991.4 Homo sapiens complement C1q A chain (C1QA), |
| | | transcript variant 1, mRNA |
| C1QA | SEQ ID NO: 84 | >NP_057075.1 complement C1q subcomponent subunit A precursor |
| | | [Homo sapiens] |
| C1QB | SEQ ID NO: 85 | >NM_000491.5 Homo sapiens complement C1q B chain (C1QB), |
| | | transcript variant 2, mRNA |
| C1QB | SEQ ID NO: 86 | >NP_000482.3 complement C1q subcomponent subunit B precursor |
| | | [Homo sapiens] |
| MYH6 | SEQ ID NO: 87 | >NM_002471.3 Homo sapiens myosin heavy chain 6 (MYH6), |
| | | mRNA |
| MYH6 | SEQ ID NO: 88 | >NP_002462.2 myosin-6 [Homo sapiens] |
| SPARC | SEQ ID NO: 89 | >NM_003118.4 Homo sapiens secreted protein acidic and cysteine |
| | | rich (SPARC), transcript variant 1, mRNA |
| SPARC | SEQ ID NO: 90 | >NP_003109.1 SPARC isoform 1 precursor [Homo sapiens] |
| TMA7 | SEQ ID NO: 91 | >NM_015933.6 Homo sapiens translation machinery associated 7 |
| | | homolog (TMA7), transcript variant 1, mRNA |
| TMA7 | SEQ ID NO: 92 | >NP_057017.1 translation machinery-associated protein 7 isoform 1 |
| | | [Homo sapiens] |
| RPL23A | SEQ ID NO: 93 | >NM_000984.6 Homo sapiens ribosomal protein L23a (RPL23A), |
| | | mRNA |
| RPL23A | SEQ ID NO: 94 | >NP_000975.2 60S ribosomal protein L23a [Homo sapiens] |
| NDUFA1 | SEQ ID NO: 95 | >NM_004541.4 Homo sapiens NADH: ubiquinone oxidoreductase |
| | | subunit A1 (NDUFA1), mRNA |
| NDUFA1 | SEQ ID NO: 96 | >NP_004532.1 NADH dehydrogenase [ubiquinone] 1 alpha |
| | | subcomplex subunit 1 [Homo sapiens] |
| COX7C | SEQ ID NO: 97 | >NM_001867.3 Homo sapiens cytochrome c oxidase subunit 7C |
| | | (COX7C), mRNA |
| COX7C | SEQ ID NO: 98 | >NP_001858.1 cytochrome c oxidase subunit 7C, mitochondrial |
| | | precursor [Homo sapiens] |
| FTH1 | SEQ ID NO: 99 | >NM_002032.3 Homo sapiens ferritin heavy chain 1 (FTH1), mRNA |
| FTH1 | SEQ ID NO: 100 | >NP_002023.2 ferritin heavy chain [Homo sapiens] |
| RPS15A | SEQ ID NO: 101 | >NM_001019.5 Homo sapiens ribosomal protein S15a (RPS15A), |
| Variant 2 | | transcript variant 2, mRNA |
| RPS15A | SEQ ID NO: 102 | >NP_001025180 ribosomal protein S15a (RPS15A), transcript |
| Variant 2 | | variant 2 [Homo sapiens] |

(h) Methods of Detecting Biomarker(s) in a Location in a Sample

Any of the exemplary methods described herein can be used to determine a level and/or at least one activity of one or more biomarkers (e.g., one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) in a location in a sample (e.g., a heart tissue sample). In some embodiments, determining a level and/or an activity of one or more biomarkers (e.g., one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) can include any of the workflows described herein.

In some embodiments, the methods can include contacting the sample with a binding agent that specifically binds to a biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) (e.g., gDNA, mRNA, a protein, or a byproduct, degredation product, or fragment, or precursor thereof), wherein the binding agent further comprises an oligonucleotide having a sequence; and sequencing all or a portion of the sequence of the oligonucleotide or a complement thereof, from a probe specifically bound to the biomarker (e.g., one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) in the location of the sample, to determine the level of the biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) in the location in the sample.

In some embodiments, the methods can include contacting a sample (e.g., a tissue sample, for instance, affixed to a support) with a plurality of probes, wherein a probe of the plurality of probes includes a protein that specifically binds to a biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to associate presence or abundance of the biomarker with the location of the tissue sample.

In some embodiments, the methods can include contacting a sample (e.g., a tissue sample, for instance, affixed to a support) with a plurality of probes, wherein a probe of the plurality of probes includes a first oligonucleotide that specifically binds to a biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) (e.g., gDNA, mRNA, or a byproduct, degredation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to associate presence or abundance of the biomarker with the location of the tissue sample.

In some embodiments, the methods can include contacting a tissue sample with a plurality of probes, wherein at least one probe of the plurality of probes comprises a protein that specifically binds to a biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) (e.g., a protein, or a byproduct, degredation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to associate presence or abundance of the biomarker with the location of interest of the tissue sample. In some embodiments, the methods can include contacting a tissue sample with a plurality of probes, wherein at least one probe of the plurality of probes comprises a first oligonucleotide that specifically binds a biomarker (e.g., one of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof) (e.g., gDNA, mRNA, or a byproduct, degredation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to associate presence or abundance of the biomarker with the location of interest of the tissue sample.

(i) Methods of (1) Diagnosing Myocardial Infarction and Identifying Increased Likelihood of Having a Myocardial Infarction; (2) Monitoring the Risk of Having a Myocardial Infarction; (3) Identifying a Candidate Drug Target (4) Determining the Efficacy of a Treatment for Reducing the Risk of Having a Myocardial Infarction; and (5) Treating Myocardial Infarction in a Subject

Provided herein are methods of diagnosing a subject as having a heart disease (e.g., a myocardial infarction). Also provided herein are methods of identifying a subject as having an increased likelihood of having a heart disease (e.g., a myocardial infarction). Further provided herein are methods of monitoring the risk of having a heart disease (e.g., a myocardial infarction). Also provided herein are methods for determining the efficacy of a treatment for reducing the risk of having a heart disease (e.g., a myocardial infarction) in a subject. Further provided herein are methods for treating a heart disease (e.g., a myocardial infarction) in a subject.

In any of these methods, a biological sample can be any appropriate biological sample obtained from the subject. In some embodiments, a biological sample can be a sample comprising blood, serum, or plasma. In some embodiments, a biological sample can be a tissue sample. In some embodiments, the method can further include obtaining the sample from the subject.

(1) Methods of Diagnosing Myocardial Infarction and Identifying Increased Likelihood of Having a Myocardial Infarction

Provided herein are methods of diagnosing a subject as having a heart disease (e.g., a myocardial infarction) and methods of identifying a subject as having an increased likelihood of having a heart disease (e.g., a myocardial infarction). In some embodiments, the methods can include: (a) determining a level of one or more of: RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, in a biological sample from a subject; and (b) identifying a subject having decreased level(s) of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, in the biological sample as compared to reference level(s) of the one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, and/or identifying a subject having increased level(s) of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof in the biological sample as compared to reference level(s) of the one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, as having a heart disease (e.g., myocardial infarction, or having an increased likelihood of having a heart disease (e.g., myocardial infarction).

In some embodiments, the methods can further include confirming a diagnosis of myocardial infarction in the subject. Non-limiting examples of ways to confirm a diagnosis of myocardial infarction include an electrocardiogram (ECG), holter monitoring, echocardiogram, stress test, cardiac catheterization, cardiac computerized tomography (CT) scan, or cardiac magnetic resonance imaging (MM).

In some embodiments, the method of confirming a diagnosis of myocardial infarction is electrocardiogram (ECG). An ECG records electrical signals and can help detect irregularities in the heart's rhythm and structure. An ECG can be performed while at rest or while exercising (stress electrocardiogram).

In some embodiments, the method of confirming a diagnosis of myocardial infarction is Holter monitoring. A Holter monitor is a portable device one wears to record a continuous ECG, usually for 24 to 72 hours. Holter monitoring is used to detect heart rhythm irregularities that aren't found during a regular ECG exam. In some embodiments, the method of confirming a diagnosis of myocardial infarction can include performing a coronary angiogram or a chest radiograph.

In some embodiments, the method of confirming a diagnosis of a heart disease or disorder is echocardiogram. This noninvasive exam, includes generating an ultrasound image of a subject's chest.

In some embodiments, the method of confirming a diagnosis of myocardial infarction includes the performance of a stress test on the subject. This type of test involves raising a subject's heart rate with exercise or medicine while performing heart tests and imaging to check how the heart responds.

In some embodiments, the method of confirming a diagnosis of myocardial infarction includes the performance of cardiac catheterization. In this test, a short tube (sheath) is inserted into a vein or artery in a subject's leg, groin, or arm. A hollow, flexible, and longer tube (guide catheter) is then inserted into the sheath. Aided by X-ray images on a monitor, a physician threads the guide catheter through that artery until it reaches the heart. The pressures in the heart chambers can be measured, and dye can be injected. The dye can be seen on an X-ray, which helps the physician see the blood flow through the heart, and blood vessels and valves of the heart.

In some embodiments, the method of confirming a diagnosis of myocardial infarction includes performing a cardiac computerized tomography (CT) scan on the subject. In a cardiac CT scan, the subject lies on a table inside a doughnut-shaped machine. An X-ray tube inside the machine rotates around the body and collects images of the heart and chest.

In some embodiments, the method of confirming a diagnosis of myocardial infarction includes the performance of cardiac magnetic resonance imaging (MRI). For this test, the subject lies on a table inside a long tube-like machine that produces a magnetic field. The magnetic field produces images to help the doctor evaluate the heart.

In some embodiments, the methods can further include performing one or more tests to further determine the subject's risk of having a myocardial infarction. Non-limiting examples of more tests to further determine the subject's risk of having a myocardial infarction include taking a family history (e.g., where a family history of myocardial infarction is indicative of an increased risk of having a myocardial infarction), detecting a genetic mutation associated with risk of having a myocardial infarction, determining the levels of other biomarkers (e.g., in blood or a component thereof) indicative an increased risk of having a myocardial infarction, and taking a health history (e.g., where a history of heart injury and/or heart disorders are indicative of an increased risk of having a myocardial infarction, or the subject's behavior indicate an increased risk of having a myocardial infarction).

In some embodiments, the methods can further include updating the subject's clinical record with the diagnosis of myocardial infarction or to indicate an increased risk of having a myocardial infarction. In some embodiments, the methods can further include enrolling the subject in a clinical trial. In some embodiments, the clinical trial could be for prevention and/or reducing the risk of having a myocardial infarction. In some embodiments, the methods can further include informing the subject's family of the diagnosis, or of the subject's likelihood of having a myocardial infarction. In some embodiments, the methods can further include assessing or referring the subject for enrollment in a supportive care plan or care facility. In some embodiments, the methods can further include monitoring the subject more frequently.

(2) Methods of Monitoring the Risk of Having a Myocardial Infarction in a Subject Over Time

Also provided herein are methods of monitoring risk of having a myocardial infarction in a subject over time that include: (a) determining a first level of one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, in a first biological sample obtained from a subject at a first time point; (b) determining a second level of one or more of the analytes of step (a), in a second biological sample obtained from the subject at a second time point; (c) identifying: (i) a subject having increased second level(s) of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof as compared to the first level(s) of the one or more of the analytes of step (a), and/or decreased second level(s) of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof as compared to the first level(s) of the one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, as having an increasing risk of having a myocardial infarction, or (ii) a subject having about the same or decreased second level(s) of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof as compared to the first level(s) of the one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, and/or about the same or increased second level(s) of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof as compared to the first level(s) of the one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof, as having about the same or a decreasing risk of having a myocardial infarction.

In some embodiments, the methods can include identifying a subject having an increasing risk of having a myocardial infarction over time (e.g., between the first and second time points). In some embodiments, when the methods include identifying a subject as having an increasing risk of having a myocardial infarction, the methods can further include administering a treatment for reducing the risk of having a myocardial infarction to the subject or increasing the dose of a previously administered treatment for reducing the risk of having a myocardial infarction to the subject. In some embodiments, the methods can further include selecting a treatment for reducing the risk of having a myocardial infarction for the subject.

In some embodiments, when the methods include identifying a subject as having about the same or a decreasing risk of having a myocardial infarction over time (e.g., between the first and second time periods). Some embodiments of these methods can further include recording in the subject's clinical record that the subject has about the same or a decreasing risk of having a myocardial infarction over time. In some embodiments, the methods can further include maintaining the dose or lowering the dose of a treatment for reducing the risk of having a myocardial infarction to be administered to the subject or ceasing administration of a treatment for reducing the risk of having a myocardial infarction to the subject.

(3) Methods of Identifying a Candidate Drug Target

Provided herein are methods for identifying a candidate drug target for treatment of a heart disorder. The methods can include (a) determining level(s) of one or more biomarker(s) in a location in a sample comprising heart tissue obtained from an animal having a heart disorder, (b) identifying: (i) one or more biomarker(s) selected from (e.g., one or more analytes): selected from (1) RPL17, (2) RPL36A, (3) TMSB4X, (4) CCDC80, (5) FTL, (6) RPL37, (7) HMOX1, (8) YBX1, (9) RPS17, (10) RPS29, (11) PPDPF, (12) YBX3, (13) VIM, (14) NDUFB1, (15) RPL34, (16) MIF, (17) PLTP, (18) RPL38, and byproducts, degradation products, or precursors thereof) showing elevated level(s) in the location in the sample as compared to reference level(s), and/or (ii) one or more biomarker(s) (e.g., one or more analytes) selected from (1) RPL17, (2) RPL36A, (3) TMSB4X, (4) CCDC80, (5) FTL, (6) RPL37, (7) HMOX1, (8) YBX1, (9) RPS17, (10) RPS29, (11) PPDPF, (12) YBX3, (13) VIM, (14) NDUFB1, (15) RPL34, (16) MIF, (17) PLTP, (18) RPL38, and byproducts, degradation products, or precursors thereof showing decreased level(s) in the location in the sample as compared to reference level(s), as candidate drug target(s) for treatment of the heart disorder. In some embodiments, a reference level of the one or more biomarker(s) is a level of the one or more biomarker(s) in a corresponding location in a sample comprising heart tissue obtained from a control animal. In some embodiments, an animal can be any of the exemplary animals described herein. In some embodiments, an animal can be a mammal.

In some embodiments, the methods can include identifying one or more biomarker(s) (e.g., one or more analytes) selected from RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, degradation products, or precursors thereof showing elevated level(s) in the location in the sample as compared to reference level(s) as candidate drug target(s) for treatment of the heart disorder. In some embodiments, the methods further include testing the ability of an inhibitor of the expression and/or activity of the one or more identified candidate drug target(s) (e.g., one or more analytes) selected from RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, degradation products, or precursors thereof) to treat the heart disorder in an animal (e.g., using a clinical trial, enzymatic assays, assessment of cell signaling activity, in vitro assays, ex vivo assays, or an animal model of the heart disorder (e.g., any of the exemplary animal models of heart disorders described herein or known in the art).

In some embodiments, the methods can include identifying one or more biomarker(s) showing decreased level(s) (e.g., one or more analytes) selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof in the location in the sample as compared to reference level(s) of the one or more biomarker(s) as candidate drug target(s) for treatment of the heart disorder. In some embodiments, the method can further include testing the ability of an agent that increases the expression and/or activity of the one or more identified candidate drug target(s) (e.g., one or more analytes) selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof) to treat the heart disorder in an animal (e.g., using a clinical trial, enzymatic assays, assessment of cell signaling activity, in vitro assays, ex vivo assays, or an animal model of the heart disorder (e.g., any of the exemplary animal models of heart disorders described herein or known in the art).

In some embodiments, the methods can further include additional studies to further validate a candidate drug target. Non-limiting examples of additional studies can include generation of a knockout (e.g., a RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, or FTH1 knockout) or a knock-in (e.g., a RPL17, RPL36A, TMSB4X, CCDC80, FTL, RPL37, HMOX1, YBX1, RPS17, RPS29, PPDPF, YBX3, VIM, NDUFB1,

RPL34, MIF, PLTP, or RPL38 knock-in) animal, administration of an agent that activates (e.g., activates one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, and RPL38) and/or inhibits (e.g., inhibits one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, and FTH1) the candidate drug target (or a protein or nucleic acid that is downstream of the activity of the candidate drug target in a cell).

Some embodiments of these methods can further include screening for a molecule that inhibits the expression and/or at least one activity of a candidate drug target (for a candidate drug target that has a level that is elevated at a location in the heart as compared to a reference level) (e.g., one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, and FTH1). Some embodiments of these methods can further include screening for a molecule that increases the expression and/or at least one activity of a candidate drug target (for a candidate drug target that has a level that is decreased at a location in the heart as compared to a reference level) (e.g., one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, and RPL38).Other studies to further validate a candidate drug target will be apparent to those skilled in the field.

(4) Methods of Determining the Efficacy of a Treatment for Reducing the Risk of Having a Myocardial Infarction in a Subject

Provided herein is a method of determining efficacy of a treatment for reducing the risk of having a myocardial infarction in a subject, wherein the method comprises: (a) determining a first level of one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, in a first biological sample obtained from a subject at a first time point; (b) determining a second level of the one or more of the analytes of step (a), in a second biological sample obtained from the subject at a second time point, wherein the subject is administered one or more doses of a treatment for reducing the risk of having a myocardial infarction between the first and second time points; and (c) identifying: (i) the treatment as being effective in a subject having about the same or decreased second level(s) of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof as compared to the first level(s) of the one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, and/or having an increased second level(s) of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof as compared to the first level(s) of the one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof; or (ii) the treatment as not being effective in a subject having increased second level(s) of one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, as compared to the first level(s) of the one or more of RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof, and/or having about the same or decreased second level(s) of one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof as compared to the first level(s) of the one or more of RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, and byproducts, precursors, and degradation products thereof.

In some embodiments, the methods include identifying the treatment as being effective in the subject. In some embodiments, the methods can further include selecting additional doses of the treatment for the subject. In some embodiments, the methods can further include administering additional doses of the treatment to the subject. In some embodiments, the methods can further include recording in the subject's clinical record that the treatment is effective in the subject.

In some embodiments, the methods include identifying the treatment as not being effective in the subject. In some embodiments, the methods can further include selecting a different treatment for the subject. In some embodiments, the methods can further include administering a different treatment to the subject. In some embodiments, the methods can further include increasing the dose of the treatment to be administered to the subject. In some embodiments, the methods can include administering one or more additional doses of the treatment to the subject in combination with an additional treatment. In some embodiments, the methods can further include ceasing administration of the therapeutic treatment to the subject. In some embodiments, the methods can further include recording in the subject's clinical record that the treatment is not effective in the subject. In some embodiments, the methods can further include referring the patient for enrollment in a clinical trial of a different treatment.

In some embodiments, the methods can further include additional assessments of the efficacy of the treatment. Non-limiting examples of additional assays that can be performed to further assess efficacy of the treatment include: obtaining an image of the subject's heart using electrocardiogram (ECG), Holter monitoring, echocardiogram, stress test, cardiac catheterization, cardiac computerized tomography (CT) scan, and cardiac magnetic resonance imaging (MRI).

(5) Methods of Treating Myocardial Infarction in a Subject

Provided herein is a method of treating a heart disease (e.g., myocardial infarction) in a subject in need thereof. In some instances, a method of treating heart disease in a subject in need thereof comprises administering an effective amount of a therapeutic agent to the subject, wherein the subject has been identified by profiling expression levels of a plurality of analytes, wherein an analyte in the plurality of analytes is correlated with the heart disease in a biological sample obtained from the subject, wherein the profile is generated from a library, wherein the library is generated by: (a) contacting the biological sample with an substrate comprising a plurality of attached capture probes, wherein a capture probe of the plurality comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

In some embodiments, the methods can further include selecting a treatment for the subject. In some embodiments, the methods can further include administering a treatment for reducing the risk of having a myocardial infarction to the subject. In some embodiments, a treatment for reducing the risk of having a myocardial infarction can be a treatment that reduces the rate of progression of the risk of having a myocardial infarction in a subject. In some embodiments, a treatment for reducing the risk of having a myocardial infarction can include lifestyle changes, such as stopping smoking, treatments to lower the blood pressure, treatments to lower the level of cholesterol, treatment of diabetes, exercise (e.g., at least 30 minutes most days of the week), change of diet (e.g., eating a low-fat and low-sodium diet, and limiting alcohol), weight control, stress management or treatment (e.g., muscle relaxation and deep breathing), depression management, and heart procedures (e.g., angioplasty), surgery (e.g., bypass surgery), or implanting a device (e.g., a pacemaker or a defibrillator).

In some embodiments, a treatment for myocardial infarction, treatment for reducing the risk of having a myocardial infarction, and/or preventing or treating a heart disorder/disease can include administering one or more therapeutic or prophylactic medications. Such medications include, but not limited to, high blood pressure medications (e.g., vasodilators, diuretics, beta-blockers, ACE inhibitors, angiotensin II receptor blockers, calcium channel blockers, alpha-2 receptor agonists, central agonists, peripheral adrenergic inhibitors, etc), cholesterol-lowering medications (including but not limited to statins, bile acid binding resins, cholesterol absorption inhibitors, fibrates, niacin, omega-3 fatty acids, combination cholesterol absorption inhibitor and statin, combination stain and calcium channel blocker, monoclonal antibodies), anti-coagulant medications (including but not limited to apixaban (Eliquis), dabigatran (Pradaxa), edoxaban (Savaysa), fondaparinux (Arixtra), heparin (Fragmin, Innohep, and Lovenox), rivaroxaban (Xarelto), and warfarin (Coumadin, Jantoven), anti-platelet medications (including but not limited to aspirin, clopidogrel (Plavix),dipyridamole (Persantine), ticlopidine (Ticlid), prasugrel (Effient), ticagrelor (Brilinta), vorapaxar (Zontivity), thrombolytic medications (including but not limited to alteplase (Activase) and streptokinase (Streptase), nitroglycerin, and pain-relievers (including but not limited to hydrocortisone (Cortef), methylprednisolone (Medrol), prednisolone (Prelone), prednisone (Deltasone), buprenorphine (Buprenex, Butrans), fentanyl (Duragesic), hydrocodone-acetaminophen (Vicodin), hydromorphone (Exalgo ER), meperidine (Demerol), oxycodone (OxyContin), oxymorphone (Opana), tramadol (Ultram), duloxetine (Cymbalta) and venlafaxine (Effexor XR), fluoxetine (Prozac) and paroxetine (Paxil), imipramine (Tofranil), nortriptyline (Pamelor), desipramine (Norpramin), carbamazepine (Tegretol), gabapentin (Neurontin), phenytoin (Dilantin), and pregabalin (Lyrica)).

In some instances, vasodilators include but are not limited to Bumetanide (Bumex), Chlorthalidone (Hygroton), Chlorothiazide (Diuril), Ethacrynate (Edecrin), Furosemide (Lasix), Hydrochlorothiazide HCTZ (Esidrix, Hydrodiuril, Microzide), Indapamide (Lozol), Methyclothiazide (Enduron), Metolazone (Mykroz, Zaroxolyn), Torsemide (Demadex), Minoxidil (Loniten), and Hydralazine (Apresoline).

In some instances, diurectics provided herein include but are not limited to Chlorthalidone (Hygroton), Chlorothiazide (Diuril), Hydrochlorothiazide or HCTZ (Esidrix, Hydrodiuril, Microzide), Indapamide (Lozol), Metolazone (Mykrox, Zaroxolyn), Amiloride (Midamor), Bumetanide (Bumex), Furosemide (Lasix), Spironolactone (Aldactone), and Triamterene (Dyrenium).

In some instances, beta-blockers provided herein include but are not limited to acebutolol (Sectral®), atenolol (Tenormin®), bisoprolol (Zebeta®), metoprolol (Lopressor®, Toprol XL®), nadolol (Corgard®), nebivolol (Bystolic®), propranolol (Inderal, InnoPran XL), carvedilol (Coreg), esmilol (Brevibloc), labetalol (Trandate, Normodyne), metoprolol tartrate (Lopressor) and metoprolol succinate (Toprol-XL), nadolol (Corgard), penbutolol sulfate (Levatol), sotalol (Betapace), and hydrochlorothiazide HCTZ and bisoprolol (Ziac).

In some instances, ACE inhibitors provided herein include but are not limited to benazepril (Lotensin®), captopril (Capoten®), enalapril (Vasotec®), fosinopril (Monopril®), lisinopril (Prinivil®, Zestril®), moexioril (Univasc®), perinopril (Aceon®), quinapril (Accupril®), ramipril (Altace®), and trandolapril (Mavik®).

In some instances, calcium channel blockers provided herein include but are not limited to Amlodipine besylate (Norvasc, Lotrel), Clevidipine (Cleviprex), Diltiazem hydrochloride (Cardizem CD, Cardizem SR, Dilacor XR, Tiazac), Felodipine (Plendil), Isradipine (DynaCirc, DynaCirc CR), Nicardipine (Cardene SR), Nifedipine (Adalat CC, Procardia XL), Nimodipine (Nimotop, Nymalize), Nisoldipine (Sular), Verapamil hydrochloride (Calan SR, Isoptin SR, Verelan, and Covera HS).

In some instances, alpha-2 receptor agonists provided herein include but are not limited to Methyldopa (Aldomet), Clonidine (Catapres®), Clonidine patch (Catapres-TTS®), Tizanidine (Zanaflex®), Clonidine (Kapvay®), Guanfacine (Intuniv®), and Lofexidine (Lucemyra™)

In some instances, central agonists provided herein include but are not limited to clonidine hydrochloride (Catapres) and guanfacine hydrochloride (Tenex).

In some instances, peripheral adrenergic inhibitors provided herein include but are not limited to guanadrel (Hylorel), guanethidine monosulfate (Ismelin), and reserpine (Serpasil).

In some instances, Angiotensin II Receptor Blockers provided herein include but are not limited to azilsartan (Edarbi), candesartan (Atacand), eprosartan, mesylate (Teveten), irbesarten (Avapro), losartin potassium (Cozaar), olmesartan (Benicar), telmisartan (Micardis), and valsartan (Diovan).

In some instances, blood thinners provided herein include but are not limited to warfarin and dabigatran.

In some instances, statins provided herein include but are not limited to atorvastatin (Lipitor), fluvastatin (Lescol XL), lovastatin (Altoprev), pitavastatin (Livalo), pravastatin (Pravachol), and rosuvastatin (Crestor), and simvastatin (Zocor).

In some instances, bile acid binding resins provided herein include but are not limited to cholestyramine (Prevalite), colesevelam (Welchol), and colestipol (Colestid).

In some instances, cholesterol absorption inhibitors provided herein include but are not limited to ezetimibe (Zetia).

In some instances, fibrates provided herein include but are not limited to fenofibrate (Antara, Lipofen), and gemfibrozil (Lopid).

In some instances, omega-3 fatty acids provided herein include but are not limited to lovaza, and icosapent ethyl (Vascepa).

In some instances, combination statin and calcium channel blockers provided herein include but are not limited to amlodipine-atorvastatin (Caduet).

In some instances, monoclonal antibodies provided herein include but are not limited to Alirocumab (Praluent) and Evolocumab (Repatha).

(j) Kits

In some embodiments, also provided herein are kits that include one or more reagents to detect a level of one or more of any of the biomarkers and/or candidate biomarkers described herein (e.g., one or more analytes selected from RPL17, RPL36A, TMSB4X, CCDC80, FTL, RPL37, HMOX1, YBX1, RPS17, RPS29, PPDPF, YBX3, VIM, NDUFB1, RPL34, MIF, PLTP, RPL38, RPS21, RPS10, RNASE1, RPS25, RPL26, RPL37A, RPS15A, EEF1A2, RPS27, NEXN, COL1A1, RPL23, COL3A1, ATP5F1E, RPS8, RPL31, PABPC1, RPS28, FABP4, DCN, MGP, RPL22, RPL39, C1QA, C1QB, MYH6, SPARC, TMA7, RPL23A, NDUFA1, COX7C, FTH1, and byproducts, precursors, and degradation products thereof.

In some embodiments, reagents can include one or more antibodies (and/or antigen-binding antibody fragments), labeled hybridization probes, and primers. For example, in some embodiments, an antibody (and/or antigen-binding antibody fragment) can be used for visualizing one or more features of a tissue sample (e.g., by using immunofluorescence or immunohistochemistry). In some embodiments, an antibody (and/or antigen-binding antibody fragment) can be an analyte binding moiety, for example, as part of an analyte capture agent. For example, in some embodiments, a kit can include an anti-RPL17 antibody, such as Prodcut No. ab155781 (Abcam). Other useful commercially available antibodies will be apparent to one skilled in the art.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit can further include instructions for performing any of the methods or steps provided herein.

### EXAMPLES

Identifying individual cells and their genetic makeup can be important for understanding their roles in how the heart physiologically functions, develops, and organizes; as well as how these modalities are altered in diseased states. The Example described herein demonstrate, e.g., the ability to do one or more of the following: (1) examine histological and transcriptome profiles from the same tissue section at a much higher resolution, better sensitivity, and shorter time; (2) obtain unbiased and high-throughput gene expression analysis for intact tissue sections across different heart regions; (3) generate spatial clustering that reliably correlates with the heart anatomy; and (4) demonstrate the ability to discover novel targets and/or pathways with unbiased analysis.

### Example 1. Spatial Gene Expression Characterization in a Human Heart Tissue Section

Spatial analysis was performed on a human heart tissue section.

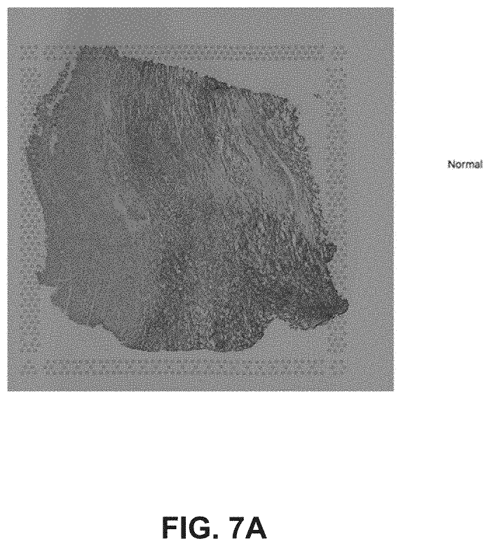

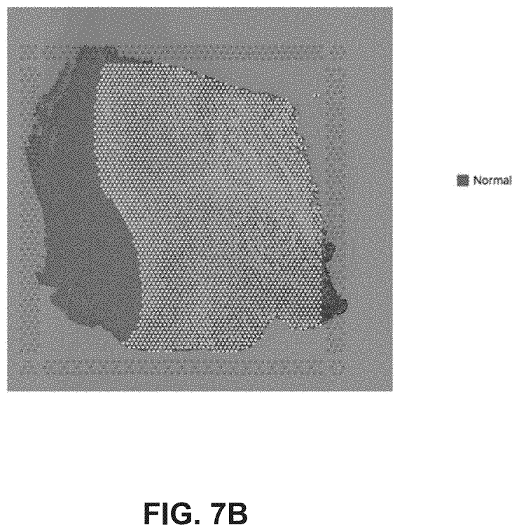

Briefly, a heart tissue sample was isolated from a human subject who had a myocardial infarction (MI). A sample was fixed with methanol, stained with hematoxylin and eosin (H&E), and imaged under a brightfield microscope. As shown in FIG. 7A, the human heart section contains both normal cardiac tissue (left part of sample) and a myocardial infarction (MI) region (right part of sample).

The sample was then permeabilized and analytes (i.e., mRNA analytes) were captured on an array comprising a plurality of capture probes. In order to capture the analytes, capture probes were designed to have poly(T) sequences that hybridize indiscriminately to the poly(A) tail of an mRNA analyte. Array features corresponding to normal vs. MI cardiac tissue were assigned to clusters based on disease state. FIG. 7B depicts an overlay of the cluster assignments with the H&E stained image. Array features assigned to the normal tissue cluster are shown in blue. Array features assigned to the MI tissue cluster are shown in white. As shown in Table 2 and Table 3 below, differential expression of genes in the normal and diseased region of the tissue section was detected. Overexpression and underexpression of various genes were detected, with statistical significance.

TABLE 2
Underexpressed genes in the MI region of human heart tissue

| | | Average | Fold Change | |
|---|---|---|---|---|
| | | Expression | in MI | |
| | Gene | in Normal | Region | Normal |
| FeatureID | Name | Region | (Log²) | p-Value |
| ENSG00000265681 | RPL17 | 1.219324 | 1.615581 | 9.92E−17 |
| ENSG00000241343 | RPL36A | 1.959874 | 1.319739 | 8.97E−12 |
| ENSG00000205542 | TMSB4X | 6.727971 | 1.237921 | 1.19E−10 |
| ENSG00000091986 | CCDC80 | 1.808 | 1.223178 | 4.53E−10 |
| ENSG00000087086 | FTL | 40.30796 | 1.158549 | 1.94E−09 |
| ENSG00000145592 | RPL37 | 6.135788 | 1.140309 | 3.69E−09 |
| ENSG00000100292 | HMOX1 | 2.906783 | 1.15301 | 3.80E−09 |
| ENSG00000065978 | YBX1 | 2.582339 | 1.135346 | 6.98E−09 |
| ENSG00000182774 | RPS17 | 1.174662 | 1.126026 | 1.60E−08 |
| ENSG00000213741 | RPS29 | 5.113351 | 1.077542 | 3.40E−08 |
| ENSG00000125534 | PPDPF | 1.293099 | 1.087845 | 4.99E−08 |
| ENSG00000060138 | YBX3 | 2.681133 | 1.071862 | 5.44E−08 |
| ENSG00000026025 | VIM | 3.784476 | 1.054252 | 8.65E−08 |
| ENSG00000183648 | NDUFB1 | 1.202722 | 1.066506 | 9.46E−08 |
| ENSG00000109475 | RPL34 | 4.490302 | 1.043326 | 9.81E−08 |
| ENSG00000240972 | MIF | 1.199916 | 1.053215 | 1.96E−07 |
| ENSG00000100979 | PLTP | 1.228911 | 1.034273 | 2.53E−07 |
| ENSG00000172809 | RPL38 | 3.571571 | 1.010484 | 2.90E−07 |

TABLE 3
Overexpressed genes in the MI region of human heart tissue

| | | | Fold | |
|---|---|---|---|---|
| | | | Change in | |
| | | | Normal | |
| | Gene | Normal | Region | Normal |
| FeatureID | Name | Average | (Log²) | p-Value |
| ENSG00000171858 | RPS21 | 3.324877 | 0.994652 | 4.80E−07 |
| ENSG00000124614 | RPS10 | 2.212298 | 0.991199 | 6.03E−07 |
| ENSG00000129538 | RNASE1 | 1.474436 | 0.98697 | 8.90E−07 |
| ENSG00000118181 | RPS25 | 3.59928 | 0.969995 | 9.87E−07 |
| ENSG00000161970 | RPL26 | 4.275643 | 0.950003 | 1.75E−06 |
| ENSG00000197756 | RPL37A | 7.861712 | 0.943593 | 1.97E−06 |
| ENSG00000134419 | RPS15A | 3.839193 | 0.94283 | 2.21E−06 |
| ENSG00000101210 | EEF1A2 | 1.268546 | 0.954216 | 2.51E−06 |
| ENSG00000177954 | RPS27 | 7.551064 | 0.933072 | 2.63E−06 |
| ENSG00000162614 | NEXN | 1.083233 | 0.954925 | 2.76E−06 |
| ENSG00000108821 | COL1A1 | 5.906865 | 0.934438 | 3.19E−06 |
| ENSG00000125691 | RPL23 | 2.772562 | 0.919656 | 4.49E−06 |
| ENSG00000168542 | COL3A1 | 6.409607 | 0.917603 | 4.54E−06 |
| ENSG00000124172 | ATP5F1E | 4.816032 | 0.921102 | 4.55E−06 |
| ENSG00000142937 | RPS8 | 5.886521 | 0.912174 | 4.68E−06 |
| ENSG00000071082 | RPL31 | 3.504578 | 0.911135 | 5.20E−06 |
| ENSG00000070756 | PABPC1 | 1.924332 | 0.920045 | 5.67E−06 |
| ENSG00000233927 | RPS28 | 6.14935 | 0.900907 | 6.29E−06 |
| ENSG00000170323 | FABP4 | 1.55581 | 0.914232 | 6.44E−06 |
| ENSG00000011465 | DCN | 2.252049 | 0.877427 | 1.64E−05 |
| ENSG00000111341 | MGP | 2.841309 | 0.875675 | 1.64E−05 |
| ENSG00000116251 | RPL22 | 2.31343 | 0.87484 | 1.64E−05 |
| ENSG00000198918 | RPL39 | 5.732659 | 0.861949 | 1.95E−05 |
| ENSG00000173372 | C1QA | 1.34501 | 0.881589 | 2.07E−05 |
| ENSG00000173369 | C1QB | 1.169751 | 0.859253 | 3.43E−05 |
| ENSG00000197616 | MYH6 | 8.501831 | 0.838432 | 3.52E−05 |
| ENSG00000113140 | SPARC | 4.746584 | 0.838531 | 3.80E−05 |
| ENSG00000232112 | TMA7 | 1.362079 | 0.842958 | 4.52E−05 |
| ENSG00000198242 | RPL23A | 3.819902 | 0.817423 | 6.24E−05 |
| ENSG00000125356 | NDUFA1 | 1.715519 | 0.821671 | 6.61E−05 |
| ENSG00000127184 | COX7C | 2.607476 | 0.815573 | 6.71E−05 |
| ENSG00000167996 | FTH1 | 40.12312 | 0.810237 | 6.71E−05 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A method of determining abundance of two or more analytes in a subject having a heart disease or disorder, comprising:
determining the abundance of the two or more analytes selected from the group consisting of ribosomal protein L36a (RPL36A), ferritin light chain (FTL), ribosomal protein L37 (RPL37), ribosomal protein S17 (RPS17), ribosomal protein S29 (RPS29), pancreatic progenitor cell differentiation and proliferation factor (PPDPF), Y-box binding protein 3 (YBX3), ribosomal protein L34 (RPL34), ribosomal protein L38 (RPL38), ribosomal protein S21 (RPS21), ribosomal protein S10 (RPS10), ribosomal protein S25 (RPS25), ribosomal protein L26 (RPL26), ribosomal protein L37a (RPL37A), ribosomal protein S15a (RPS15A), ribosomal protein S27 (RPS27), ribosomal protein L23 (RPL23), ATP synthase F1 subunit epsilon (ATP5F1E), ribosomal protein S8 (RPS8), ribosomal protein L31 (RPL31), ribosomal protein S28 (RPS28), fatty acid binding protein 4 (FABP4), ribosomal protein L22 (RPL22), ribosomal protein L39 (RPL39), complement C1q B chain (C1QB), translation machinery associated 7 homolog (TMA7), ribosomal protein L23a (RPL23A), NADH:ubiquinone oxidoreductase subunit A1 (NDUFA1), cytochrome c oxidase subunit 7C (COX7C), ferritin heavy chain 1 (FTH1), and byproducts, precursors, and degradation products thereof, in a biological sample obtained from the subject.

3. The method of claim 2, wherein the biological sample from the subject comprises more than one biological sample from the subject from a plurality of time points and determining the abundance of the two or more analytes in the two or more biological samples from the plurality of time points from the subject.

4. (canceled)

5. A method of treating heart disease or disorder in a subject, wherein the method comprises:
(a) determining an abundance of two or more analytes selected from RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, in a biological sample from the subject; and
(b) identifying the subject having at least one of:
(1) an elevated abundance of the two or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof, of step (a), as compared to the reference expression level(s) of the two or more analytes RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1 and byproducts, precursors, and degradation products thereof; and
(2) about the same or a decreased abundance of the two or more analytes RPL36A, FTL, RPL37, RPS17, RPS29, PPDPF, YBX3, RPL34, RPL38, and byproducts, precursors, and degradation products thereof, of step (a), as compared to the reference expression level(s) of the two or more analytes RPL36A, FTL, RPL37, RPS17, RPS29, PPDPF, YBX3, RPL34, RPL38, and byproducts, precursors, and degradation products thereof;
as having a heart disease or disorder; and
(c) administering a treatment for heart disease or disorder to the subject.

6. The method of claim 5, wherein the heart disease or disorder is myocardial infarction.

7. (canceled)

8. The method of claim 6, wherein the method further comprises confirming a diagnosis of myocardial infarction in the subject by obtaining an image of the subject's heart or performing cardiac testing on the subject.

9-14. (canceled)

15. The method of claim 5, wherein the treatment is
(a) an antagonist of one or more analytes RPS21, RPS10, RPS25, RPL26, RPL37A, RPS15A, RPL23, ATP5F1E, RPS8, RPL31, RPS28, FABP4, RPL22, RPL39, C1QA, C1QB, TMA7, RPL23A, NDUFA1, COX7C, FTH1, and byproducts, precursors, and degradation products thereof; or
(b) an agonist of one or more analytes RPL36A, FTL, RPL37, RPS17, RPS29, PPDPF, YBX3, RPL34, RPL38, and byproducts, precursors, and degradation products thereof.

16-17. (canceled)

18. The method of claim 2, further comprising administering a treatment to the subject for reducing the risk of having a heart disease or disorder, adjusting a dosage of a treatment for the subject for reducing the risk of having a heart disease or disorder, or adjusting a treatment for the subject for reducing the risk of having a heart disease or disorder.

19. The method of claim 5, wherein the treatment comprises administering at least one medication selected from a blood pressure lowering drug, a cholesterol lowering drug, an anti-coagulant drug, an anti-platelet drug, a thrombolytic drug, nitroglycerin, and a pain-reliever.

20. The method of claim 19, wherein
(a) the blood pressure lowering drug is one or more drugs selected from the group consisting of a diuretic, a beta-blocker, an ACE inhibitor, an angiotensin II receptor blocker, a calcium channel blocker, an alpha blocker, an alpha-2 receptor agonist, a central agonist, a peripheral adrenergic inhibitor, and a vasodilator;
(b) the cholesterol lowering drug is one or more drugs selected from the group consisting of a statin, a bile acid binding resin, a cholesterol absorption inhibitor, a fibrate, niacin, an omega-3 fatty acid, a combination cholesterol absorption inhibitor and statin, a combination stain and calcium channel blocker, and a monoclonal antibody;
(c) the anti-coagulant drug is one or more drugs selected from the group consisting of apixaban (Eliquis), dabigatran (Pradaxa), edoxaban (Savaysa), fondaparinux (Arixtra), heparin (Fragmin, Innohep, and Lovenox), rivaroxaban (Xarelto), and warfarin (Coumadin, Jantoven);
(d) the anti-platelet drug is one or more drugs selected from the group consisting of aspirin, clopidogrel (Plavix), dipyridamole (Persantine), ticlopidine (Ticlid), prasugrel (Effient), ticagrelor (Brilinta), vorapaxar (Zontivity);
(e) the thrombolytic drug is one or more drugs selected from the group consisting of alteplase (Activase) and streptokinase (Streptase); and
(f) the pain-reliever drug is one or more drugs selected from the group consisting of hydrocortisone (Cortef), methylprednisolone (Medrol), prednisolone (Prelone), prednisone (Deltasone), buprenorphine (Buprenex, Butrans), fentanyl (Duragesic), hydrocodone-acetaminophen (Vicodin), hydromorphone (Exalgo ER), meperidine (Demerol), oxycodone (OxyContin), oxymorphone (Opana), tramadol (Ultram), duloxetine (Cymbalta) and venlafaxine (Effexor XR), fluoxetine (Prozac) and paroxetine (Paxil), imipramine (Tofranil), nortriptyline (Pamelor), desipramine (Norpramin), carbamazepine (Tegretol), gabapentin (Neurontin), phenytoin (Dilantin), and pregabalin (Lyrica).

21. The method of claim 20, wherein
(a) the vasodilator is one or more drugs selected from the group consisting of Bumetanide (Bumex), Chlorthalidone (Hygroton), Chlorothiazide (Diuril), Ethacrynate (Edecrin), Furosemide (Lasix), Hydrochlorothiazide HCTZ (Esidrix, Hydrodiuril, Microzide), Indapamide (Lozol), Methyclothiazide (Enduron), Metolazone (Mykroz, Zaroxolyn), Torsemide (Demadex), Minoxidil (Loniten), and Hydralazine (Apresoline);
(b) the diuretic is one or more drugs selected from the group consisting of Chlorthalidone (Hygroton), Chlorothiazide (Diuril), Hydrochlorothiazide or HCTZ (Esidrix, Hydrodiuril, Microzide), Indapamide (Lozol), Metolazone (Mykrox, Zaroxolyn), Amiloride (Midamor), Bumetanide (Bumex), Furosemide (Lasix), Spironolactone (Aldactone), and Triamterene (Dyrenium);
(c) the beta-blocker is one or more drugs selected from the group consisting of acebutolol (Sectral®), atenolol (Tenormin®), bisoprolol (Zebeta®), metoprolol (Lopressor®, Toprol XL®), nadolol (Corgard®), nebivolol (Bystolic®), propranolol (Inderal, InnoPran XL), carvedilol (Coreg), esmilol (Brevibloc), labetalol (Trandate, Normodyne), metoprolol tartrate (Lopressor) and metoprolol succinate (Toprol-XL), nadolol (Corgard), penbutolol sulfate (Levatol), sotalol (Betapace), and hydrochlorothiazide HCTZ and bisoprolol (Ziac);
(d) the ACE inhibitor is one or more drugs selected from the group consisting of benazepril (Lotensin®), captopril (Capoten®), enalapril (Vasotec®), fosinopril (Monopril®), lisinopril (Prinivil®, Zestril®), moexioril (Univasc®), perinopril (Aceon®), quinapril (Accupril®), ramipril (Altace®), and trandolapril (Mavik®);
(e) the angiotensin II receptor blocker is one or more drugs selected from the group consisting of azilsartan (Edarbi), candesartan (Atacand), eprosartan, mesylate (Teveten), irbesarten (Avapro), losartin potassium (Cozaar), olmesartan (Benicar), telmisartan (Micardis), and valsartan (Diovan);
(f) the calcium channel blocker is one or more drugs selected from the group consisting of Amlodipine besylate (Norvasc, Lotrel), Clevidipine (Cleviprex), Diltiazem hydrochloride (Cardizem CD, Cardizem SR, Dilacor XR, Tiazac), Felodipine (Plendil), Isradipine (DynaCirc, DynaCirc CR), Nicardipine (Cardene SR), Nifedipine (Adalat CC, Procardia XL), Nimodipine (Nimotop, Nymalize), Nisoldipine (Sular), Verapamil hydrochloride (Calan SR, Isoptin SR, Verelan, and Covera HS);
(g) the alpha-2 receptor agonist is one or more drugs selected from the group consisting of Methyldopa (Aldomet), Clonidine (Catapres®), Clonidine patch (Catapres-TTS®), Tizanidine (Zanaflex®), Clonidine (Kapvay®), Guanfacine (Intuniv®), and Lofexidine (Lucemyra™);
(h) the central agonist is one or more drugs selected from the group consisting of clonidine hydrochloride (Catapres) and guanfacine hydrochloride (Tenex);
(i) the peripheral adrenergic inhibitor is one or more drugs selected from the group consisting of guanadrel (Hylorel), guanethidine monosulfate (Ismelin), and reserpine (Serpasil);
(j) the statin is one or more drugs selected from the group consisting of atorvastatin (Lipitor), fluvastatin (Lescol XL), lovastatin (Altoprev), pitavastatin (Livalo), pravastatin (Pravachol), and rosuvastatin (Crestor), and simvastatin (Zocor);
(k) the bile acid binding resin is one or more drugs selected from the group consisting of cholestyramine (Prevalite), colesevelam (Welchol), and colestipol (Colestid);
(l) the cholesterol absorption inhibitor is ezetimibe (Zetia);
(m) the fibrates is one or more drugs selected from the group consisting of fenofibrate (Antara, Lipofen), and gemfibrozil (Lopid);
(n) the omega-3 fatty acids is one or more drugs selected from the group consisting of lovaza, and icosapent ethyl (Vascepa);
(o) the combination statin and calcium channel blocker is amlodipine-atorvastatin (Caduet); and
(p) the monoclonal antibody is one or more antibodies selected from the group consisting of Alirocumab (Praluent) and Evolocumab (Repatha).

22. The method of claim 19, wherein the treatment further comprises angioplasty, undergoing bypass surgery, and implanting a pacemaker or a defibrillator.

23. (canceled)

24. The method of claim 2, wherein the biological sample comprises blood, serum, plasma, or tissue.

25. The method of claim 2, wherein the biological sample comprises cardiac tissue.

26. The method of claim 2, wherein the two or more analytes are mRNA molecules.

27. The method of claim 26, wherein the determining step comprises:
(a) contacting the biological sample with a substrate comprising a plurality of attached capture probes, wherein a capture probe of the plurality comprises (i) a spatial barcode and (ii) a capture domain that binds to a sequence present in the analyte;
(b) hybridizing the two or more analytes to the capture domain;
(c) extending a 3′ end of the capture probe using the analyte that is bound to the capture domain as a template to generate an extended capture probe;
(d) amplifying the extended capture probe; and
(e) determining (i) all or a portion of the sequence of the spatial barcode or the complement thereof, and (ii) all or a portion of the sequence of the analyte from the biological sample; and using the determined sequences of (i) and (ii) to identify the location of the analyte in the biological sample, thereby determining the abundance and location of the two or more analytes.

28. (canceled)

29. The method of claim 2, wherein the heart disease or disorder is selected from the group consisting of: congenital heart disease or disorder, arrhythmia, tachycardia, bradycardia, premature ventricular contraction, fibrillation, coronary artery disease (CAD), heart muscle disease, dilated cardiomyopathy, myocardial infarction, heart failure, hypertrophic cardiomyopathy, mitral regurgitation, heart valve disease, mitral valve prolapse, pulmonary stenosis, pericardial disease, heart infection, aneurysm, and sudden cardiac arrest.

30. The method of claim 2, wherein the two or more analytes are proteins.

31. The method of claim 30, wherein the determining step comprises determining the abundance and location of the two or more analytes, the method comprising:
(a) attaching the biological sample with a plurality of analyte capture agents, wherein an analyte capture agent of the plurality of analyte capture agents comprises:
(i) an analyte binding moiety that binds to the two or more analytes
(ii) an analyte binding moiety barcode that uniquely identifies an interaction between the two or more analytes and the analyte binding moiety; and
(iii) an analyte capture sequence, wherein the analyte capture sequence binds to a capture domain;
(b) contacting the biological sample with a substrate, wherein the substrate comprises a plurality of capture probes, wherein a capture probe of the plurality of capture probes comprises (i) the capture domain and (ii) a spatial barcode;
(c) hybridizing the two or more analytes to the capture probe; and
(d) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the two or more analytes in the biological sample.

32-39. (canceled)

40. A kit comprising:
(a) an antibody that binds specifically to two or more analytes selected from the group consisting of RPL36A, FTL, RPL37, RPS17, RPS29, PPDPF, YBX3, RPL34, RPL38, RPS21, RPS10, RPS25, RPL26, RPL37A, RPS15A, RPS27, RPL23, ATP5F1E, RPS8, RPL31, RPS28, FABP4, RPL22, RPL39, C1QB, TMA7, RPL23A, NDUFA1, COX7C, FTH1, and byproducts, precursors, and degradation products thereof; and
(b) instructions for performing the method of claim 27.
